(19) United States
(12) Patent Application Publication
(10) Pub. No.: US 2025/0095775 A1
(43) Pub. Date: Mar. 20, 2025

(54) METHODS FOR DETERMINING VARIANT FREQUENCY AND MONITORING DISEASE PROGRESSION

(71) Applicant: Foundation Medicine, Inc., Boston, MA (US)

(72) Inventors: Mark KENNEDY, Cambridge, MA (US); Wai-Ki YIP, Brookline, MA (US); Doron LIPSON, Chestnut Hill, MA (US); Jonathan FREIDIN, Cambridge, MA (US)

(73) Assignee: Foundation Medicine, Inc., Boston, MA (US)

(21) Appl. No.: 18/291,495

(22) PCT Filed: Jun. 8, 2022
(86) PCT No.: PCT/US2022/032725

§ 371 Date: Jan. 23, 2024

Related U.S. Application Data
(60) Provisional application No. 63/225,397, filed on Jul. 23, 2021.

(51) Int. Cl. G16B 20/20; C12Q 1/6827; C12Q 1/6858; G16B 30/10; G16H 50/20

(52) U.S. Cl. CPC G16B 20/20; C12Q 1/6827; C12Q 1/6858; G16B 30/10; G16H 50/20

(57) ABSTRACT

Methods for determining a variant frequency in a test sample from a subject, and methods for labeling sequencing reads as having or not having a variant are described herein. Exemplary methods include generating a reference match score and a variant match score by aligning sequencing reads to a corresponding variant sequence and a corresponding reference sequence, and labeling the sequencing read as having or not having the variant based on the determined match scores. Also described herein are methods monitoring disease progression and methods of treating a subject having a disease. Further described are devices and systems for implementing such methods.

### CROSS-REFERENCE TO RELATED APPLICATIONS

This application claims the priority benefit of U.S. Provisional Application No. 63/225,397 filed on Jul. 23, 2021, the contents of which are incorporated herein by reference in its entirety.

### FIELD OF THE INVENTION

Methods and systems for identifying a variant, determining a variant frequency in a test sample, methods of monitoring disease progression (such as cancer progression) and methods of treating a subject with a disease (such as cancer) are described herein.

### BACKGROUND

Genomic testing shows significant promise towards developing better understanding of cancers and managing more effective treatment approaches. Genomic testing involves the sequencing of the genome, or a portion thereof, of a patient's biological sample (which may contain cancer cells or cell-free nucleic acid products of cancer cells) and identifying any genetic variants (for example, a mutation that may be associated with a tumor) in the sample versus a reference genetic sequence. A genetic variant can include, for example, insertions, deletions, substitutions, rearrangements, or any combination thereof. Identifying and understanding these genetic variants (e.g., mutations) as they are found in a specific patient's cancer may also help develop better treatments and help identify the best approaches (or exclude ineffective approaches) for treating specific cancer variants using genomic information.

Generally, biological samples are processed in a laboratory with various possible techniques, with the end goal of extracting and isolating DNA contained therein. That isolated DNA is sequenced, resulting in a data structure representation (which may be electronic) of the DNA from the patient sample. Often, that data structure representation is in the form of several thousand “reads” or more (e.g., tens of thousands, hundreds of thousands, millions, tens of millions, or hundreds of millions reads). A single read generally comprises a relatively short (e.g., 50-150 bases) subsequence of the patient's DNA. In contrast, the entire human genome is approximately 3 billion bases long, and sub-regions of interest for the purposes of this application can be several tens of thousands bases long.

Diseases, such as cancer and clonal hematopoiesis, can be monitored or determined in a patient by determining variant frequency among nucleic acid molecules in a sample taken from the patient. Cancer severity is generally correlated with the number of variants within the tumor genome or the relative frequency at which those variants appear in a sample. For example, cell-free DNA is generally a mixture of genomic DNA and circulating-tumor DNA. As the severity of the cancer increases, a larger portion of the cell-free DNA is attributable to the cancer. By tracking the relative frequency of variants indicative of the tumor genome, progression of the disease can be monitored.

Variant calling processes generally require a threshold number of sequencing reads to be identified as having the variant before a positive variant call is made. Detecting a sufficient number of sequencing reads often requires substantial sequencing depth, which may not be possible if only limited amounts of disease-associated nucleic acid is available. There remains a need for efficient variant calling processes that have a low limit of detection and can be used for tracking disease progression.

Variant calling processes may include noise introduced in sequencing reads during a sequencing and alignment process in the variant calling process. As a result of potential errors associated with sequencing data, sequencing reads may be incorrectly identified as alternate (e.g., variant) when the variant is not present in the sample data. That is, these errors can result in false positives—where the sequencing read is identified as variant, when in fact, the variant is not present in the sequencing read. Accordingly, there remains a need to implement variant calling methods that can account for noise and improve accuracy while not requiring a high limit of detection.

### BRIEF SUMMARY OF THE INVENTION

Described herein are methods of detecting a genetic variant and determining a variant allele frequency in a sample from a subject. Also described herein are methods of monitoring disease progression and methods of treating a subject with a disease. Further described are electronic devices and systems for carrying out such methods.

An exemplary method of detecting a genetic variant or determining a variant allele frequency in a sample from a subject comprises providing a plurality of nucleic acid molecules obtained from the sample, ligating one or more adapters onto one or more nucleic acid molecules from the plurality of nucleic acid molecules, amplifying the one or more ligated nucleic acid molecules from the plurality of nucleic acid molecules, capturing the amplified nucleic acid molecules from the amplified nucleic acid molecules, sequencing, by a sequencer, the captured nucleic acid molecules to obtain a plurality of sequencing reads that represent the nucleic acid molecules, wherein one or more of the plurality of sequencing reads overlap a variant locus of the genetic variant, generating, using one or more processors, a reference match score for each of the one or more sequencing reads by aligning each of the one or more sequencing reads to a reference sequence that does not comprise the genetic variant, generating, using the one or more processors, a variant match score for each of the one or more sequencing reads by aligning each sequencing read to a variant sequence that comprises the genetic variant, based on the reference match score and the variant match score of a respective sequencing read, labeling, using the one or more processors, each of the one or more sequencing reads as at least one of having the genetic variant, not having the genetic variant, or being an inconclusive read, determining, using the one or more processors, a number of sequencing reads labeled as having the genetic variant in the plurality of sequencing reads, determining, using the one or more processors, a probability metric based on a variant specific model, the number of sequencing reads labeled as having the genetic variant, and a total number of labeled sequencing reads, and identifying, using the one or more processors, the presence of the genetic variant in the sample when the determined probability metric is less than a first threshold.

In some embodiments, the variant specific model is locus specific. In some embodiments, the first threshold is locus specific and variant specific. In some embodiments, the probability metric is a statistical value indicative of a likelihood that the genetic variant is detected due to the presence of the genetic variant in the sample rather than noise. In some embodiments, the method further comprises comparing, using the one or more processors, the determined probability metric to a second threshold, and identifying, by the one or more processors, the absence of the genetic variant in the sample if the determined probability metric is greater than or equal to the second threshold, or identifying, by the one or more processors, the presence or absence of the genetic variant in the sample as inconclusive if the determined probability metric is greater than or equal to the first threshold and less than the second threshold.

In some embodiments, the subject is suspected of or is determined to have cancer. In some embodiments, the method further comprises obtaining the sample from the subject. In some embodiments, the sample comprises a tissue biopsy sample, a liquid biopsy sample, or a normal control. In some embodiments, the sample is a liquid biopsy sample and comprises blood, plasma, cerebrospinal fluid, sputum, stool, urine, or saliva. In some embodiments, the sample is a liquid biopsy sample and comprises cell-free DNA (cfDNA), circulating tumor DNA (ctDNA), or any combination thereof. In some embodiments, the plurality of nucleic acid molecules comprises a mixture of tumor nucleic acid molecules and non-tumor nucleic acid molecules. In some embodiments, the tumor nucleic acid molecules are derived from a tumor portion of a heterogeneous tissue biopsy sample, and the non-tumor nucleic acid molecules are derived from a normal portion of the heterogeneous tissue biopsy sample.

In some embodiments, the sample comprises a liquid biopsy sample, and wherein the tumor nucleic acid molecules are derived from a circulating tumor DNA (ctDNA) fraction of the liquid biopsy sample, and the non-tumor nucleic acid molecules are derived from a non-tumor, cell-free DNA (cfDNA) fraction of the liquid biopsy sample. In some embodiments, the one or more adapters comprise amplification primers, flow cell adaptor sequences, substrate adapter sequences, or sample index sequences. In some embodiments, the captured nucleic acid molecules are captured from the amplified nucleic acid molecules by hybridization to one or more bait molecules. In some embodiments, the one or more bait molecules comprise one or more nucleic acid molecules, each comprising a region that is complementary to a region of a captured nucleic acid molecule. In some embodiments, amplifying nucleic acid molecules comprises performing a polymerase chain reaction (PCR) amplification technique, a non-PCR amplification technique, or an isothermal amplification technique. In some embodiments, the sequencing comprises use of a next generation sequencing (NGS) technique, whole genome sequencing (WGS), whole exome sequencing, targeted sequencing, direct sequencing, or Sanger sequencing technique. In some embodiments, the sequencer comprises a next generation sequencer. In some instances, a minimum sequencing coverage of at least 75×, 100×, 150×, 150×, 200×, or 250× is required.

In some embodiments, the plurality of sequencing reads comprises between 100 and 3,000 loci, between 200 and 2,800 loci, between 300 and 2,600 loci, between 400 and 2,400 loci, between 500 and 2,200 loci, between 600 and 2,000 loci, between 700 and 1,800 loci, between 800 and 1,600 loci, between 900 and 1,400 loci, between 1,000 and 1,200 loci, between 400 and 1,000 loci, between 400 and 1,200 loci, between 400 and 1,400 loci, between 400 and 1,600 loci, between 400 and 1,800 loci, between 400 and 2,000 loci, between 400 and 2,200 loci, between 400 and 2,400 loci, between 400 and 2,600 loci, between 400 and 2,800 loci, between 400, and 3,000 loci, between 600 and 1,000 loci, between 600 and 1,200 loci, between 600 and 1,400 loci, between 600 and 1,600 loci, between 600 and 1,800 loci, between 600 and 2,000 loci, between 600 and 2,200 loci, between 600 and 2,400 loci, between 600 and 2,600 loci, between 600 and 2,800 loci, between 600, and 3,000 loci, between 800 and 1,000 loci, between 800 and 1,200 loci, between 800 and 1,400 loci, between 800 and 1,600 loci, between 800 and 1,800 loci, between 800 and 2,000 loci, between 800 and 2,200 loci, between 800 and 2,400 loci, between 800 and 2,600 loci, between 800 and 2,800 loci, between 800, and 3,000 loci, between 1,000 and 1,200 loci, between 1,000 and 1,400 loci, between 1,000 and 1,600 loci, between 1,000 and 1,800 loci, between 1,000 and 2,000 loci, between 1,000 and 2,200 loci, between 1,000 and 2,400 loci, between 1,000 and 2,600 loci, between 1,000 and 2,800 loci, between 1,000, and 3,000 loci, between 1,200 and 1,400 loci, between 1,200 and 1,600 loci, between 1,200 and 1,800 loci, between 1,200 and 2,000 loci, between 1,200 and 2,200 loci, between 1,200 and 2,400 loci, between 1,200 and 2,600 loci, between 1,200 and 2,800 loci, between 1,200, and 3,000 loci, between 1,400 and 1,600 loci, between 1,400 and 1,800 loci, between 1,400 and 2,000 loci, between 1,400 and 2,200 loci, between 1,400 and 2,400 loci, between 1,400 and 2,600 loci, between 1,400 and 2,800 loci, between 1,400, and 3,000 loci, between 1,600 and 1,800 loci, between 1,600 and 2,000 loci, between 1,600 and 2,200 loci, between 1,600 and 2,400 loci, between 1,600 and 2,600 loci, between 1,600 and 2,800 loci, between 1,600, and 3,000 loci, between 1,800 and 2,000 loci, between 1,800 and 2,200 loci, between 1,800 and 2,400 loci, between 1,800 and 2,600 loci, between 1,800 and 2,800 loci, between 1,800, and 3,000 loci, between 2,000 and 2,200 loci, between 2,000 and 2,400 loci, between 2,000 and 2,600 loci, between 2,000 and 2,800 loci, between 2,000 and 3,000 loci, between 2,200 and 2,400 loci, between 2,200 and 2,600 loci, between 2,200 and 2,800 loci, between 2,200, and 3,000 loci, between 2,400 and 2,600 loci, between 2,400 and 2,800 loci, between 2,400, and 3,000 loci, between 2,600 and 2,800 loci, between 2,600, and 3,000 loci, or between 2,800 and 3,000 loci.

In some embodiments, the method further comprises generating, by the one or more processors, a report indicating the presence of the genetic variant in the sample. In some instances, the report comprises output from the method described herein. In some embodiments, the report is transmitted to, e.g., a healthcare provider, over the Internet via a computer network or peer-to-peer connection. In some instances, the method further comprises displaying the report in a data field on a display device. In some instances, the method further comprises displaying a user interface comprising the report or output from the method via an online portal. In some instances, the method further comprises displaying a user interface comprising the report or output from the method via a mobile device.

An exemplary method of detecting a genetic variant in a sample from a subject comprises obtaining a plurality of sequencing reads associated with the sample, wherein one or more of the plurality of sequencing reads that overlap a variant locus associated with the genetic variant, generating, by one or more processors, a reference match score for each of the plurality of sequencing reads by aligning each of the one or more sequencing reads to a reference sequence that does not comprise the genetic variant, generating, by the one or more processors, a variant match score for each of the plurality of sequencing reads by aligning each sequencing read to a variant sequence that comprises the genetic variant, labeling, by the one or more processors, each of the plurality of sequencing reads as at least one of having the genetic variant, not having the genetic variant, or being an inconclusive read based on the reference match score and the variant match score of the respective sequencing read, determining, by the one or more processors, a number of sequencing reads labeled as having the genetic variant in the plurality of sequencing reads, determining, by the one or more processors, a probability metric based on a variant specific model, the number of sequencing reads labeled as having the genetic variant, and a total number of labeled sequencing reads, and identifying, by the one or more processors, the presence of the genetic variant in the sample when the determined probability metric is less than a first threshold.

In some embodiments, the variant specific model is locus specific. In some embodiments, the first threshold is locus specific and variant specific. In some embodiments, the probability metric corresponds to a probability that the genetic variant is detected due to the presence of the genetic variant in the sample rather than noise. In some embodiments, the method further comprises comparing, using the one or more processors, the determined probability metric to a second threshold, and identifying, by the one or more processors, the absence of the genetic variant in the sample if the determined probability metric is greater than or equal to the second threshold, or identifying, by the one or more processors, the presence or absence of the genetic variant in the sample as inconclusive if the determined probability metric is greater than or equal to the first threshold and less than the second threshold. In some embodiments, the variant specific model is generated by fitting, using the one or more processors, a probability distribution based on a determined metric and a total number of labeled sequencing reads from a wild-type sample. In some embodiments, the probability distribution is a binomial distribution. In some embodiments, the probability metric is determined from the number of sequencing reads labeled as having the genetic variant and a second number, wherein the second number is the total number of labeled sequencing reads minus a number of sequencing reads labeled as being inconclusive reads. In some embodiments, the variant specific model is associated with one or more functions related to one of more sources of noise in a plurality of sequencing reads that overlap the variant locus. In some embodiments, the one or more sources of noise comprise sample preparation errors, amplification bias errors, sequencing errors, alignment errors, or any combination thereof. In some embodiments, the variant specific model is associated with one or more functions that have been fitted to data for a plurality of sequencing reads that overlap the variant locus. In some embodiments, the one or more functions comprise one or more of uniform distribution functions, binomial distribution functions, Poisson distribution functions, negative binomial distribution functions, normal distribution functions, log-normal distribution functions, Cauchy-Lorentz distribution functions, log-logistic distribution functions, exponential distribution functions, gamma distribution functions, hypergeometric distribution functions, or any combination thereof.

In some embodiments, a sequencing read is labeled as having the genetic variant if the reference match score and the variant match score indicate that the sequencing read more closely matches the variant sequence than the reference sequence. In some embodiments, a sequencing read is labeled as not having the genetic variant if the reference match score and the variant match score indicate that the sequencing read more closely matches the reference sequence than the variant sequence. In some embodiments, a sequencing read is labeled as the inconclusive read if the reference match score and the variant match score are equal.

In some embodiments, the first threshold is determined empirically using the variant specific model. In some embodiments, at least one of the first threshold or the second threshold is determined empirically using clinical trial outcomes. In some embodiments, the first threshold is determined using a Kaplan-Meier estimator and data associated with samples from a plurality of subjects. In some embodiments, the second threshold is determined empirically using the variant specific model, and is set to a value that corresponds to a specified confidence level that a sequencing read labeled as not containing the genetic variant is correct.

In some embodiments, the reference sequence and the variant sequence comprise the variant locus, a 5′ flanking region, and a 3′ flanking region. In some embodiments, 5′ flanking region and 3′ flanking region are each about 5 bases in length to about 5000 bases in length. In some embodiments, the method further comprises generating from the sample, the variant sequence.

In some embodiments, generating the variant sequence comprises providing a plurality of nucleic acid molecules obtained from the sample, ligating one or more adapters onto one or more nucleic acid molecules from the plurality of nucleic acid molecules, amplifying the one or more ligated nucleic acid molecules from the plurality of nucleic acid molecules, capturing the amplified nucleic acid molecules from the amplified nucleic acid molecules, and sequencing, by a sequencer, the captured nucleic acid molecules to obtain a plurality of sequencing reads that represent the nucleic acid molecules, wherein one or more of the plurality of sequencing reads overlap a variant locus of the genetic variant. In some embodiments, the reference sequence and the variant sequence are substantially identical except for the genetic variant.

In some embodiments, the method further comprises determining a variant allele frequency for the genetic variant using the number of sequencing reads labeled as having the genetic variant and the number of sequencing reads labeled as not having the genetic variant. In some embodiments, the method further comprises labeling sequencing reads associated with the sample for a second genetic variant selected from the one or more variants, determining a probability metric using a second variant specific model, the number of sequencing reads labeled as having the second genetic variant and a total number of labeled sequencing reads for the second genetic variant, and comparing the determined probability metric for the second genetic variant to a corresponding third threshold, wherein if the determined probability metric for the second genetic variant is less than the third threshold, the presence of the second genetic variant in the sample is identified. In some embodiments, the second genetic variant is associated with a second variant locus selected from the one or more variants. In some embodiments, the method further comprises comparing the determined probability metric for the second genetic variant to a fourth threshold, when the determined probability metric is greater than or equal to the fourth threshold, identifying the absence of the second genetic variant in the sample, and when the determined probability metric is greater than or equal to the third threshold and less than the fourth threshold, the presence or absence of the second genetic variant in the sample is inconclusive.

In some embodiments, the method further comprises determining a disease status for the subject. In some embodiments, the disease status is a value proportional to a percentage of circulating-tumor DNA (ctDNA) compared to total cell-free DNA (cfDNA) in the sample. In some embodiments, the disease status is a maximum somatic allele fraction of cfDNA. In some embodiments, the disease status comprises a qualitative factor indicating recurrence of a cancer in the subject, the presence of a cancer resistant to a treatment modality in the subject, or the presence of a cancer that can be treated with a particular treatment modality. In some embodiments, the sample comprises cfDNA. In some embodiments, the reference match score and the variant match score are determined using a sequence alignment algorithm. In some embodiments, the sequence alignment algorithm is at least one of a Smith-Waterman alignment algorithm, a Striped Smith-Waterman alignment algorithm, or a Needleman-Wunsch alignment algorithm. In some embodiments, the genetic variant comprises a single nucleotide variant (SNV), a multiple nucleotide variant (MNV), an indel, or a rearrangement junction. In some embodiments, the variant panel is determined by sequencing nucleic acid molecules in a previous sample obtained from the subject, and identifying one or more genetic variants.

In some embodiments, the subject received an intervening treatment for a disease between the previous sample being obtained and the sample being obtained. In some embodiments, the disease is cancer. In some embodiments, the cancer is a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of the oral cavity, cancer of the pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.

In some embodiments, the method further comprises adjusting the treatment based on a difference between a disease status for the subject determined using the sample and a previous disease status for the subject based on the previous sample. In some embodiments, the method further comprises generating the one or more sequencing reads by sequencing nucleic acid molecules in the sample. In some embodiments, the variant is a somatic mutation. In some embodiments, the variant is a germline mutation.

In some embodiments, the method further comprises determining, identifying, or applying the presence of the genetic variant of the sample as a diagnostic value associated with the sample. In some instances, the determined presence of the genetic variant in the sample is used in making suggested treatment decisions for the subject. For example, the determined presence of the genetic variant in the sample may be used in suggesting an anti-cancer agent (or anti-cancer therapy, e.g., any drug that is effective in the treatment of malignant, or cancerous, disease, including, but not limited to alkylating agents, antimetabolites, natural products, and hormones), chemotherapy, radiation therapy, immunotherapy, surgery, or a therapy configured to target a the presence of the genetic variant.

In some instances, the disclosed methods for determining the presence of a genetic variant in a sample may be implemented as part of a genomic profiling process that comprises, identification of the presence of variant sequences at one or more gene loci in a sample derived from a subject as part of detecting, monitoring, predicting a risk factor, or selecting a treatment for a particular disease, e.g., cancer. In some instances, the variant panel selected for genomic profiling may comprise the detection of variant sequences at a selected set of gene loci. In some instances, the variant panel selected for genomic profiling may comprise detection of variant sequences at a number of gene loci through comprehensive genomic profiling (CGP), a next-generation sequencing (NGS) approach used to assess hundreds of genes (including relevant cancer biomarkers) in a single assay. Inclusion of the disclosed methods for determining the presence of a genetic variant in a sample as part of a genomic profiling process can improve the validity of, e.g., disease detection calls, made on the basis of the genomic profiling by, for example, independently confirming the presence of a genetic variant in a given patient sample.

In some embodiments, the method further comprises generating a genomic profile for the subject based on the presence of the genetic variant. In some embodiments, the method further comprises administering an anti-cancer agent or applying an anti-cancer treatment to the subject based on the generated genomic profile. In some embodiments, the presence of the genetic variant of the sample is used in making suggested treatment decisions for the subject. In some embodiments, the presence of the genetic variant of the sample is used in applying or administering a treatment to the subject.

In some instances, the genomic profile for the subject may further comprise results from a comprehensive genomic profiling (CGP) test, a nucleic acid sequencing-based test, a gene expression profiling test, a cancer hotspot panel test, a DNA methylation test, a DNA fragmentation test, an RNA fragmentation test, or any combination thereof. In some instances, a genomic profile may comprise information on the presence of genes (or variant sequences thereof), copy number variations, epigenetic traits, proteins (or modifications thereof), and/or other biomarkers in an individual's genome and/or proteome, as well as information on the individual's corresponding phenotypic traits and the interaction between genetic or genomic traits, phenotypic traits, and environmental factors.

In some embodiments, an exemplary method for detecting a disease state in a sample from a subject comprises sequencing nucleic acid molecules in the sample acquired from the subject to generate a plurality of sequencing reads, and detecting a genetic variant of determining a variant allele frequency in the sample according to the method described herein. In some embodiments, an exemplary method of monitoring disease progression or recurrence comprises sequencing nucleic acid molecules in a first sample acquired from a subject with a disease to generate a first set of sequencing reads, generating a personalized variant panel for the subject, sequencing nucleic acid molecules in a second sample acquired from the subject at a later time point than the first sample to generate a second set of sequencing reads, and detecting, using the second set of sequencing reads, a genetic variant or determining, using the second set of sequencing reads, a variant allele frequency according to the method described herein.

In some embodiments, the method further comprises administering a disease therapy to the subject after the first sample is acquired from the subject and before the second sample is acquired from the subject. In some embodiments, the method further comprises determining a first disease status based on a number of sequencing reads in the first set of sequencing reads labeled as having a genetic variant from the variant panel, and determining a second disease status based on a number of sequencing reads in the second set of sequencing reads labeled as having the genetic variant from the variant panel. In some embodiments, the method further comprises determining disease progression by comparing the first disease status and the second disease status. In some embodiments, the method further comprises administering a disease therapy to the subject after the first sample is acquired from the subject and before the second sample is acquired from the subject and adjusting the disease therapy based on the determined disease progression.

In some embodiments, an exemplary method of treating a subject with a disease comprises acquiring a first sample from the subject, sequencing nucleic acid molecules in a first sample to generate a first set of sequencing reads, determining a first disease status using the first set of sequencing reads, generating a personalized variant panel for the subject, administering a disease therapy to the subject, acquiring a second sample from the subject after the disease therapy has been administered to the subject, sequencing nucleic acid molecules in the second sample to generate a second set of sequencing reads, detecting, using the second set of sequencing reads, a genetic variant or determining, using the second set of sequencing reads, a variant allele frequency according to the method described herein, determining a second disease status based on the second set of sequencing reads, determining disease progression by comparing the first disease status and the second disease status, adjusting the disease therapy administered to subject based on the disease progression, and administering the adjusted disease therapy to the subject. In some embodiments, the disease is cancer.

In some embodiments, the sample is derived from a liquid biopsy sample from the subject. In some embodiments, the sample is derived from a solid tissue sample, liquid tissue sample, or hematological sample, from the subject. In some embodiments, the method further comprises sequencing nucleic acid molecules extracted from the sample to generate the plurality of sequencing reads. In some embodiments, the method further comprises generating or updating a report comprising (1) identifying information for the subject, and (2) a call for the presence or absence of the genetic variant, or a call for the variant allele frequency for the genetic variant. In some embodiments, the method further comprises transmitting the report to the subject or a healthcare provider for the subject.

An exemplary apparatus comprises one or more processors, a memory, and one or more programs, wherein the one or more programs are stored in the memory and configured to be executed by the one or more processors, the one or more programs including instructions for selecting a genetic variant at a variant locus from one or more variants, obtaining a plurality of sequencing reads associated with a sample that overlap the variant locus, generating a reference match score for each of the plurality of sequencing reads by aligning each sequencing read to a reference sequence that does not comprise the genetic variant, generating a variant match score for each of the plurality of sequencing reads by aligning each sequencing read to a variant sequence that comprises the genetic variant, labeling each of the one or more sequencing reads as at least one of having the genetic variant, as not having the genetic variant, or as being an inconclusive read based on the reference match score and the variant match score of the respective sequencing read, determining a number of sequencing reads labeled as having the genetic variant, determining a probability metric based on a variant specific model and a total number of labeled sequencing reads, and identifying, using the one or more processors, the presence of the genetic variant in the sample if the determined probability metric is less than a first threshold.

In some embodiments, the variant specific model is locus specific. In some embodiments, the first threshold is locus specific and variant specific. In some embodiments, the probability metric is a statistical value indicative of a likelihood that the genetic variant is detected due to the presence of the genetic variant in the sample rather than noise. In some embodiments, the one or more programs further include instructions for comparing, using the one or more processors, the determined probability metric to a second threshold, and identifying, by the one or more processors, the absence of the genetic variant in the sample if the determined probability metric is greater than or equal to the second threshold, or identifying, by the one or more processors, the presence or absence of the genetic variant in the sample as inconclusive if the determined probability metric is greater than or equal to the first threshold and less than the second threshold.

In some embodiments, the variant specific model is generated by fitting, using the one or more processors, a probability distribution based on a determined metric and a total number of labeled sequencing reads from a wild-type sample. In some embodiments, the probability distribution is a binomial distribution. In some embodiments, the probability metric is determined from the number of sequencing reads labeled as having the genetic variant and a second number, wherein the second number is the total number of labeled sequencing reads minus a number of sequencing reads labeled as being inconclusive reads. In some embodiments, the variant specific model is associated with one or more functions related to one of more sources of noise in a plurality of sequencing reads that overlap the variant locus. In some embodiments, the one or more sources of noise comprise sample preparation errors, amplification bias errors, sequencing errors, alignment errors, or any combination thereof. In some embodiments, the variant specific model is associated with one or more functions that have been fitted to data for a plurality of sequencing reads that overlap the variant locus. In some embodiments, the one or more functions comprise one or more of uniform distribution functions, binomial distribution functions, Poisson distribution functions, negative binomial distribution functions, normal distribution functions, log-normal distribution functions, Cauchy-Lorentz distribution functions, log-logistic distribution functions, exponential distribution functions, gamma distribution functions, hypergeometric distribution functions, or any combination thereof.

In some embodiments, a sequencing read is labeled as having the genetic variant if the reference match score and the variant match score indicate that the sequencing read more closely matches the variant sequence than the reference sequence. In some embodiments, a sequencing read is labeled as not having the genetic variant if the reference match score and the variant match score indicate that the sequencing read more closely matches the reference sequence than the variant sequence. In some embodiments, a sequencing read is labeled as the inconclusive read if the reference match score and the variant match score are equal.

In some embodiments, the first threshold is determined empirically using the variant specific model. In some embodiments, at least one of the first threshold or the second threshold is determined empirically using clinical trial outcomes. In some embodiments, the first threshold is determined using a Kaplan-Meier estimator and data associated with samples from a plurality of subjects. In some embodiments, the second threshold is determined empirically using the variant specific model, and is set to a value that corresponds to a specified confidence level that a sequencing read labeled as not containing the genetic variant is correct.

In some embodiments, the reference sequence and the variant sequence comprise the variant locus, a 5′ flanking region, and a 3′ flanking region. In some embodiments, 5′ flanking region and 3′ flanking region are each about 5 bases in length to about 5000 bases in length.

In some embodiments, the one or more programs further include instructions for generating from the sample, the variant sequence. In some embodiments, generating the variant sequence comprises providing a plurality of nucleic acid molecules obtained from the sample, ligating one or more adapters onto one or more nucleic acid molecules from the plurality of nucleic acid molecules, amplifying the one or more ligated nucleic acid molecules from the plurality of nucleic acid molecules, capturing the amplified nucleic acid molecules from the amplified nucleic acid molecules, and sequencing, by a sequencer, the captured nucleic acid molecules to obtain a plurality of sequencing reads that represent the nucleic acid molecules, wherein one or more of the plurality of sequencing reads overlap a variant locus of the genetic variant. In some embodiments, the reference sequence and the variant sequence are substantially identical except for the genetic variant. In some embodiments, the one or more programs further include instructions for determining a variant allele frequency for the genetic variant using the number of sequencing reads labeled as having the genetic variant and the number of sequencing reads labeled as not having the genetic variant.

In some embodiments, the one or more programs further include instructions for labeling sequencing reads associated with the sample for a second genetic variant selected from the one or more variants, determining a probability metric using a second variant specific model, the number of sequencing reads labeled as having the second genetic variant and a total number of labeled sequencing reads for the second genetic variant, and comparing the determined probability metric for the second genetic variant to a corresponding third threshold, wherein if the determined probability metric for the second genetic variant is less than the third threshold, the presence of the second genetic variant in the sample is identified.

In some embodiments, the second genetic variant is associated with a second variant locus selected from the one or more variants. In some embodiments, the one or more programs further include instructions for comparing the determined probability metric for the second genetic variant to a fourth threshold, when the determined probability metric is greater than or equal to the fourth threshold, identifying the absence of the second genetic variant in the sample, and when the determined probability metric is greater than or equal to the third threshold and less than the fourth threshold, the presence or absence of the second genetic variant in the sample is inconclusive.

In some embodiments, the apparatus further comprises determining a disease status for the subject. In some embodiments, the disease status is a value proportional to a percentage of circulating-tumor DNA (ctDNA) compared to total cell-free DNA (cfDNA) in the sample. In some embodiments, the disease status is a maximum somatic allele fraction of cfDNA. In some embodiments, the disease status comprises a qualitative factor indicating recurrence of a cancer in the subject, the presence of a cancer resistant to a treatment modality in the subject, or the presence of a cancer that can be treated with a particular treatment modality. In some embodiments, the sample comprises cfDNA.

In some embodiments, the reference match score and the variant match score are determined using a sequence alignment algorithm. In some embodiments, the sequence alignment algorithm is at least one of a Smith-Waterman alignment algorithm, a Striped Smith-Waterman alignment algorithm, or a Needleman-Wunsch alignment algorithm. In some embodiments, the genetic variant comprises a single nucleotide variant (SNV), a multiple nucleotide variant (MNV), an indel, or a rearrangement junction. In some embodiments, the variant panel is determined by sequencing nucleic acid molecules in a previous sample obtained from the subject, and identifying one or more genetic variants. In some embodiments, the subject received an intervening treatment for a disease between the previous sample being obtained and the sample being obtained. In some embodiments, the disease is cancer. In some embodiments, the one or more programs further include instructions for adjusting the treatment based on a difference between a disease status for the subject determined using the sample and a previous disease status for the subject based on the previous sample.

In some embodiments, the one or more programs further include instructions for generating the one or more sequencing reads by sequencing nucleic acid molecules in the sample. In some embodiments, the variant is a somatic mutation. In some embodiments, the variant is a germline mutation. In some embodiments, the one or more programs further include instructions for determining, identifying, or applying the presence of the genetic variant of the sample as a diagnostic value associated with the sample. In some embodiments, the one or more programs further include instructions for generating a genomic profile for the subject based on the presence of the genetic variant. In some embodiments, the one or more programs further include instructions for administering an anti-cancer agent or applying an anti-cancer treatment to the subject based on the generated genomic profile. In some embodiments, the presence of the genetic variant of the sample is used in generating a genomic profile for the subject. In some embodiments, the presence of the genetic variant of the sample is used in making suggested treatment decisions for the subject. In some embodiments, the presence of the genetic variant of the sample is used in applying or administering a treatment to the subject.

An exemplary non-transitory computer-readable storage medium stores one or more programs, the one or more programs comprising instructions, the instructions when executed by one or more processors of an electronic device, cause the electronic device to select a genetic variant at a variant locus from one or more variants, obtain a plurality of sequencing reads associated with a sample that overlaps the variant locus, generate a reference match score for each of the plurality of sequencing reads by aligning each sequencing read to a reference sequence that does not comprise the genetic variant, generate a variant match score for each of the plurality of sequencing reads by aligning each sequencing read to a variant sequence that comprises the genetic variant, label each of the plurality of sequencing reads as at least one of having the genetic variant, as not having the genetic variant, or as being an inconclusive read based on the reference match score and the variant match score of the respective sequencing read, determine a number of sequencing reads labeled as having the genetic variant, determine a probability metric based on a variant specific model and a total number of labeled sequencing reads, and identify the presence of the genetic variant in the sample if the determined probability metric is less than a first threshold.

In some embodiments, the variant specific model is locus specific. In some embodiments, the first threshold is locus specific and variant specific. In some embodiments, the probability metric is a statistical value indicative of a likelihood that the genetic variant is detected due to the presence of the genetic variant in the sample rather than noise. In some embodiments, the one or more programs further including instructions for comparing, using the one or more processors, the determined probability metric to a second threshold, and identifying, by the one or more processors, the absence of the genetic variant in the sample if the determined probability metric is greater than or equal to the second threshold, or identifying, by the one or more processors, the presence or absence of the genetic variant in the sample as inconclusive if the determined probability metric is greater than or equal to the first threshold and less than the second threshold.

In some embodiments, the variant specific model is generated by fitting, using the one or more processors, a probability distribution based on a determined metric and a total number of labeled sequencing reads from a wild-type sample. In some embodiments, the probability distribution is a binomial distribution. In some embodiments, the probability metric is determined from the number of sequencing reads labeled as having the genetic variant and a second number, wherein the second number is the total number of labeled sequencing reads minus a number of sequencing reads labeled as being inconclusive reads. In some embodiments, the variant specific model is associated with one or more functions related to one of more sources of noise in a plurality of sequencing reads that overlap the variant locus. In some embodiments, the one or more sources of noise comprise sample preparation errors, amplification bias errors, sequencing errors, alignment errors, or any combination thereof. In some embodiments, the variant specific model is associated with one or more functions that have been fitted to data for a plurality of sequencing reads that overlap the variant locus. In some embodiments, the one or more functions comprise one or more of uniform distribution functions, binomial distribution functions, Poisson distribution functions, negative binomial distribution functions, normal distribution functions, log-normal distribution functions, Cauchy-Lorentz distribution functions, log-logistic distribution functions, exponential distribution functions, gamma distribution functions, hypergeometric distribution functions, or any combination thereof.

In some embodiments, a sequencing read is labeled as having the genetic variant if the reference match score and the variant match score indicate that the sequencing read more closely matches the variant sequence than the reference sequence. In some embodiments, a sequencing read is labeled as not having the genetic variant if the reference match score and the variant match score indicate that the sequencing read more closely matches the reference sequence than the variant sequence. In some embodiments, a sequencing read is labeled as the inconclusive read if the reference match score and the variant match score are equal.

In some embodiments, the first threshold is determined empirically using the variant specific model. In some embodiments, at least one of the first threshold or the second threshold is determined empirically using clinical trial outcomes. In some embodiments, the first threshold is determined using a Kaplan-Meier estimator and data associated with samples from a plurality of subjects. In some embodiments, the second threshold is determined empirically using the variant specific model, and is set to a value that corresponds to a specified confidence level that a sequencing read labeled as not containing the genetic variant is correct.

In some embodiments, the reference sequence and the variant sequence comprise the variant locus, a 5′ flanking region, and a 3′ flanking region. In some embodiments, 5′ flanking region and 3′ flanking region are each about 5 bases in length to about 5000 bases in length. In some embodiments, the one or more programs further comprising instructions for generating from the sample, the variant sequence. In some embodiments, generating the variant sequence comprises providing a plurality of nucleic acid molecules obtained from the sample, ligating one or more adapters onto one or more nucleic acid molecules from the plurality of nucleic acid molecules, amplifying the one or more ligated nucleic acid molecules from the plurality of nucleic acid molecules, capturing the amplified nucleic acid molecules from the amplified nucleic acid molecules, and sequencing, by a sequencer, the captured nucleic acid molecules to obtain a plurality of sequencing reads that represent the nucleic acid molecules, wherein one or more of the plurality of sequencing reads overlap a variant locus of the genetic variant. In some embodiments, the reference sequence and the variant sequence are substantially identical except for the genetic variant.

In some embodiments, the one or more programs further comprise instructions for determining a variant allele frequency for the genetic variant using the number of sequencing reads labeled as having the genetic variant and the number of sequencing reads labeled as not having the genetic variant. In some embodiments, the one or more programs further comprise instructions for labeling sequencing reads associated with the sample for a second genetic variant selected from the one or more variants, determining a probability metric using a second variant specific model, the number of sequencing reads labeled as having the second genetic variant and a total number of labeled sequencing reads for the second genetic variant, and comparing the determined probability metric for the second genetic variant to a corresponding third threshold, wherein if the determined probability metric for the second genetic variant is less than the third threshold, the presence of the second genetic variant in the sample is identified.

In some embodiments, the second genetic variant is associated with a second variant locus selected from the one or more variants. In some embodiments, the one or more programs further include instructions for comparing the determined probability metric for the second genetic variant to a fourth threshold, when the determined probability metric is greater than or equal to the fourth threshold, identifying the absence of the second genetic variant in the sample, and when the determined probability metric is greater than or equal to the third threshold and less than the fourth threshold, the presence or absence of the second genetic variant in the sample is inconclusive.

In some embodiments, the one or more programs further comprising instructions for determining a disease status for the subject. In some embodiments, the disease status is a value proportional to a percentage of circulating-tumor DNA (ctDNA) compared to total cell-free DNA (cfDNA) in the sample. In some embodiments, the disease status is a maximum somatic allele fraction of cfDNA. In some embodiments, the disease status comprises a qualitative factor indicating recurrence of a cancer in the subject, the presence of a cancer resistant to a treatment modality in the subject, or the presence of a cancer that can be treated with a particular treatment modality. In some embodiments, the sample comprises cfDNA.

In some embodiments, the reference match score and the variant match score are determined using a sequence alignment algorithm. In some embodiments, the sequence alignment algorithm is at least one of a Smith-Waterman alignment algorithm, a Striped Smith-Waterman alignment algorithm, or a Needleman-Wunsch alignment algorithm. In some embodiments, the genetic variant comprises a single nucleotide variant (SNV), a multiple nucleotide variant (MNV), an indel, or a rearrangement junction.

In some embodiments, the variant panel is determined by sequencing nucleic acid molecules in a previous sample obtained from the subject, and identifying one or more genetic variants. In some embodiments, the subject received an intervening treatment for a disease between the previous sample being obtained and the sample being obtained. In some embodiments, the disease is cancer. In some embodiments, the one or more programs further include instructions for adjusting the treatment based on a difference between a disease status for the subject determined using the sample and a previous disease status for the subject based on the previous sample.

In some embodiments, the one or more programs further comprising instructions for generating the one or more sequencing reads by sequencing nucleic acid molecules in the sample. In some embodiments, the variant is a somatic mutation. In some embodiments, the variant is a germline mutation. In some embodiments, the one or more programs further include instructions for determining, identifying, or applying the presence of the genetic variant of the sample as a diagnostic value associated with the sample. In some embodiments, the one or more programs further include instructions for generating a genomic profile for the subject based on the presence of the genetic variant. In some embodiments, the one or more programs further include instructions for administering an anti-cancer agent or applying an anti-cancer treatment to the subject based on the generated genomic profile. In some embodiments, the presence of the genetic variant of the sample is used in generating a genomic profile for the subject. In some embodiments, the presence of the genetic variant of the sample is used in making suggested treatment decisions for the subject. In some embodiments, the presence of the genetic variant of the sample is used in applying or administering a treatment to the subject.

An exemplary computer system comprises a processor, and a memory communicatively coupled to the processor, configured to store instructions that, when executed by the processor cause the processor to perform any of the methods described herein.

### BRIEF DESCRIPTION OF THE DRAWINGS

FIG. 1 shows an exemplary embodiment of a method for labeling sequencing reads.

FIG. 2 shows an example of a computing device in accordance with one embodiment.

FIG. 3 shows the variant distribution of variants in a panel for Sample 1 as further described in the examples.

FIG. 4 shows the variant distribution of variants in a panel for Sample 2 as further described in the examples.

FIG. 5 shows a plot of the number of variant reads detected using an exemplary method described herein (y-axis) against the number of variant reads detected using the standard variant calling protocol (x-axis) in log scale (left) and normalized (right) for Sample 1, as described in the examples.

FIG. 6 shows a plot of the variant locus depth at each variant locus for the sum of sequencing reads labeled as having the variant or not having the variant (i.e., excluding inconclusive reads) (y-axis) using an exemplary method described herein against the variant locus depth at each variant locus for the sum of sequencing reads from the initial pool of sequencing reads that overlap with the variant locus (x-axis) in log scale (left) and normalized (right) for Sample 1, as described in the examples.

FIG. 7 shows a plot of the number of variant reads detected using an exemplary method described herein (y-axis) against the number of variant reads detected using the standard variant calling protocol (x-axis) in log scale (left) and normalized (right) for Sample 2, as described in the examples.

FIG. 8 shows a plot of the variant locus depth at each variant locus for the sum of sequencing reads labeled as having the variant or not having the variant (i.e., excluding inconclusive reads) (y-axis) using an exemplary method described herein against the variant locus depth at each variant locus for the sum of sequencing reads from the initial pool of sequencing reads that overlap with the variant locus (x-axis) in log scale (left) and normalized (right) for Sample 2, as described in the examples.

FIG. 9A shows a plot of the number of variant reads detected using another exemplary method described herein (y-axis) against the number of variant reads detected using the standard variant calling protocol (x-axis) in log scale (left) and normalized (right) for Sample 1, as described in the examples.

FIG. 9B shows a plot of the variant locus depth at each variant locus for the sum of sequencing reads labeled as having the variant or not having the variant (i.e., excluding inconclusive reads) (y-axis) using another exemplary method described herein against the variant locus depth at each variant locus for the sum of sequencing reads from the initial pool of sequencing reads that overlap with the variant locus (x-axis) in log scale (left) and normalized (right) for Sample 1, as described in the examples.

FIG. 10A shows a plot of the number of variant reads detected using another exemplary method described herein (y-axis) against the number of variant reads detected using the standard variant calling protocol (x-axis) in log scale (left) and normalized (right) for Sample 2, as described in the examples.

FIG. 10B shows a plot of the variant locus depth at each variant locus for the sum of sequencing reads labeled as having the variant or not having the variant (i.e., excluding inconclusive reads) (y-axis) using another exemplary method described herein against the variant locus depth at each variant locus for the sum of sequencing reads from the initial pool of sequencing reads that overlap with the variant locus (x-axis) in log scale (left) and normalized (right) for Sample 2, as described in the examples.

FIG. 11 shows an exemplary method for detecting a genetic variant and determining a variant allele frequency in a sample from a subject.

FIG. 12 shows an exemplary method for determining a probability model based on a plurality of samples.

FIG. 13 shows an exemplary method for detecting a genetic variant and determining a variant allele frequency in a sample from a subject.

FIG. 14 shows an exemplary method for detecting a genetic variant and determining a variant allele frequency in a sample from a subject.

FIG. 15 shows an exemplary method for detecting a genetic variant and determining a variant allele frequency in a sample from a subject.

### DETAILED DESCRIPTION OF THE INVENTION

Described herein are methods for detecting a genetic variant and/or assessing a variant allele frequency of one or more samples obtained from a subject. Methods disclosed herein can be used in making clinical decisions when treating a subject so that the treating physician can be confident in their assessment of the subject. Sequencing nucleic acid molecules for a subject and de novo variant calling can provide useful information that can be used characterize the disease. However, nucleic acid sequencing is generally subject to substantial noise due to mutations introduced during PCR amplification, errors made during nucleotide detection during sequencing, and other anomalies that may be introduced during the sequencing process. For this reason, many sequencing pipelines require a threshold number of unique sequencing reads having the same variant before the variant is confidently called. Sequencing at sufficiently high depth can overcome this hurdle, but can be expensive and may not be possible if limited tumor nucleic acids are available (for example, in the case of circulating tumor (ctDNA) shed from a small tumor clone). Further, certain bona fide variants may be detected but not positively called because the number of detected sequencing reads having the variant does not meet the call threshold. In some embodiments, sequencing reads labeled as having a variant from a predetermined variant panel lowers the limit of detection because the likelihood of a false positive variant call from an a priori panel is unlikely due to random chance. Further, de novo variant calling is computationally expensive. The methods described herein streamline the variant calling process for generating more efficient variant calls and more efficient measurements of allele frequency of a given variant. For example, the methods described herein can be limited to the analysis of a selected number of loci.

Further still, methods described herein can be used to improve the accuracy of detecting a genetic variant or determining a variant allele frequency by accounting for noise using a model (e.g., a probability model). As discussed above, nucleic acid sequencing is susceptible to noise introduced during the sequencing, amplification, and/or alignment of a sample. As a result of potential errors associated with sequencing reads of a sample may be incorrectly identified as alternate (e.g., variant) when the variant is not present in the sequencing read. That is, errors introduced via the sequencing and alignment processes can result in false positives—where the sequencing read is identified as variant, when in fact, the variant is not present in the sequencing read. Accordingly, accounting for noise when evaluating a sample can improve the accuracy of results. Thus, as discussed with respect to methods disclosed herein, a model, e.g., a variant specific model (e.g., probability model) can be utilized to account for noise and improve accuracy when detecting a genetic variant or determining a variant allele frequency in a sample.

In some examples, the noise associated with a sequencing read can be locus specific. For example, in some embodiments, the alignment process can be sensitive to the sequence context of a variant at a variant locus. Accordingly, in some embodiments, accounting for noise associated with a sample can be locus specific. For example, in some embodiments, the model can be associated with one or more functions related to one of more sources of noise in a plurality of sequencing reads that overlap the variant locus. As noted above, the one or more sources of noise can include sample preparation errors, amplification bias errors, sequencing errors, alignment errors, or any combination thereof.

A variant specific model (e.g., a probability model) can provide a probability that the observed number of reads identified as variant indicates a true positive (e.g., real genetic variant) rather than a false positive (e.g., due to noise). The variant specific model can be generated based on a pool of samples that are known to not contain a variant of interest, e.g., reference variant. The model can be then be applied to a sample from a subject to determine a variant allele frequency, or detect the presence or absence of a variant in the sample. In some embodiments, variant allele frequency determination or variant detection can utilize a personal variant panel established for a subject using an initial sample. The personalized variant panel includes genetic variants that are indicative of the disease. The variant panel can then be used to quickly label most sequencing reads from the subject as either having or not having the variant sequence. The labeled sequencing reads can be then used to determine a disease status based on variant frequency.

In some embodiments, a method of detecting a genetic variant or determining a variant allele frequency in a sample from a subject, includes selecting the genetic variant at a variant locus from one or more variants. The method can include obtaining a plurality of sequencing reads associated with the sample that overlap the variant locus. The method can include generating, using one or more processors, a reference match score for each of the plurality of sequencing reads by aligning each sequencing read to a corresponding reference sequence that does not comprise the genetic variant and generating, using the one or more processors, a variant match score for each of the plurality of sequencing reads by aligning each sequencing read to a variant sequence that comprises the genetic variant. The method can include labeling, using the one or more processors, each of the plurality of sequencing reads as at least one of having the genetic variant, as not having the genetic variant, or as being an inconclusive read based on the reference match score and the variant match score of the respective sequencing read. The method can include determining, using the one or more processors, a number of sequencing reads labeled as having the genetic variant in the plurality of sequencing reads and determining, using the one or more processors, a probability metric based on a variant specific model and a total number of labeled sequencing reads. The method can further include identifying, using the one or more processors, the presence of the genetic variant in the sample if the determined probability metric is less than a first threshold.

In some embodiments, a method of detecting a genetic variant or determining a variant allele frequency in a sample from a subject includes providing a plurality of nucleic acid molecules obtained from a sample from a subject, wherein the plurality of nucleic acid molecules comprises a mixture of tumor nucleic acid molecules and non-tumor nucleic acid molecules. Optionally, one or more adapters can be ligated onto one or more nucleic acid molecules from the plurality of nucleic acid molecules. In some embodiments, the nucleic acid molecules from the plurality of nucleic acid molecules can be amplified. In some embodiments, nucleic acid molecules from the amplified nucleic acid molecules can be captured, wherein the captured nucleic acid molecules are captured from the amplified nucleic acid molecules by hybridization to one or more bait molecules. In some embodiments, the captured nucleic acid molecules can be sequenced, by a sequencer, to obtain a plurality of sequencing reads associated with the sample that overlap a variant locus of the genetic variant.

In some embodiments, one or more processors can generate a reference match score for each of the plurality of sequencing reads by aligning each sequencing read to a corresponding reference sequence that does not comprise the genetic variant. In some embodiments, the one or more processors can also generate a variant match score for each of the plurality of sequencing reads by aligning each sequencing read to a variant sequence that comprises the genetic variant. In some embodiments, the one or more processors can label each of the plurality of sequencing reads as at least one of having the genetic variant, as not having the genetic variant, or as being an inconclusive read based on the reference match score and the variant match score of the respective sequencing read. In some embodiments, the one or more processors can determine a number of sequencing reads labeled as having the genetic variant in the plurality of sequencing reads. In some embodiments, the one or more processors, can determine a probability metric based on a variant specific model and a total number of labeled sequencing reads. In some embodiments, the one or more processors can identify the presence of the genetic variant in the sample if the determined probability metric is less than a first threshold. Based on the identification of the presence of the genetic variant in the sample, a disease state in the sample can be detected.

The method of determining variant allele frequency can be used to monitor disease progression. For example, a method of monitoring disease progression can include sequencing nucleic acid molecules in a first test sample acquired from a subject with a disease to generate first sequencing reads; generating a personalized variant panel for the subject; sequencing nucleic acid molecules in a second test sample acquired from the subject at a later time point than the first test sample to generate second sequencing reads; and labeling the second sequencing reads using the method described herein. The labeled sequencing reads may then be used to determine a disease status for the subject, which can be compared to a previously determined disease status (e.g., a disease status associated with the subject at the time the first test sample was acquired from the subject) to monitor disease progression. In some embodiments, a variant specific model, e.g., probability model, can be applied to determine a disease status for the subject.

Disease status monitoring may further be used to treat a subject with a disease, for example by adjusting a disease therapy based on the monitored disease progression. For example, in some embodiments, a method of treating a subject with a disease may include acquiring a first test sample from the subject; sequencing nucleic acid molecules in a first test sample to generate first sequencing reads; generating a personalized variant panel for the subject; administering a disease therapy to the subject; acquiring a second test sample from the subject after the disease therapy has been administered to the subject; sequencing nucleic acid molecules in the second test sample to generate second sequencing reads; labeling the second sequencing reads using the method described herein; determining disease progression by comparing the first disease status and the second disease status; adjusting the disease therapy administered to subject based on the disease progression; and administering the adjusted disease therapy to the subject.

In some embodiments, the disease is cancer.

### Definitions

As used herein, the singular forms “a,” “an,” and “the” include the plural reference unless the context clearly dictates otherwise.

Reference to “about” a value or parameter herein includes (and describes) variations that are directed to that value or parameter per se. For example, description referring to “about X” includes description of “X”.

The terms “individual,” “patient,” and “subject” are used synonymously, and refers to an animal, such as a human.

A “reference” sequence is any sequence that is used to compare to a test or subject sequence (e.g., a sequencing read), and may be a standardized reference sequence (e.g., a sequence from a standardized reference assembly, such as GRCh38 from the Genome Reference Consortium or an alternative reference assembly) or a personalized reference sequence (e.g., a sequence from a healthy tissue of a subject).

The term “variant” refers to any sequence difference between a subject sequence and a reference sequence that is compared to the subject sequence. Accordingly, the term “variant” encompasses differences between a sequence from a healthy individual and a reference sequence that is used to identify a population variation, or a difference between a sequence from a diseased disuse (e.g., a tumor tissue) and a sequence from a healthy tissue (e.g., a mutation).

It is understood that aspects and variations of the invention described herein include “consisting” and/or “consisting essentially of” aspects and variations.

When a range of values is provided, it is to be understood that each intervening value between the upper and lower limit of that range, and any other stated or intervening value in that states range, is encompassed within the scope of the present disclosure. Where the stated range includes upper or lower limits, ranges excluding either of those included limits are also included in the present disclosure.

Some of the analytical methods described herein include mapping sequences to a reference sequence, determining sequence information, and/or analyzing sequence information. It is well understood in the art that complementary sequences can be readily determined and/or analyzed, and that the description provided herein encompasses analytical methods performed in reference to a complementary sequence.

The section headings used herein are for organization purposes only and are not to be construed as limiting the subject matter described. The description is presented to enable one of ordinary skill in the art to make and use the invention and is provided in the context of a patent application and its requirements. Various modifications to the described embodiments will be readily apparent to those persons skilled in the art and the generic principles herein may be applied to other embodiments. Thus, the present invention is not intended to be limited to the embodiment shown but is to be accorded the widest scope consistent with the principles and features described herein.

The figures illustrate processes according to various embodiments. In the exemplary processes, some blocks are, optionally, combined, the order of some blocks is, optionally, changed, and some blocks are, optionally, omitted. In some examples, additional steps may be performed in combination with the exemplary processes. Accordingly, the operations as illustrated (and described in greater detail below) are exemplary by nature and, as such, should not be viewed as limiting.

The disclosures of all publications, patents, and patent applications referred to herein are each hereby incorporated by reference in their entireties. To the extent that any reference incorporated by reference conflicts with the instant disclosure, the instant disclosure shall control.

### Variant Panels

Certain methods described herein use a variant panel that includes one or more genetic variants of interest. The genetic variants may be, for example, variants that are associated with a particular disease (e.g., cancer or cancer clone) or disease state (e.g., metastasis). In some embodiments, the variant panel is a personalized variant panel. In some embodiments, the variant panel is a diseased patient population variant panel based on variants detected in a population of subjects having a particular disease. In some embodiments, the variant panel can be a part of a comprehensive panel that screens for multiple diseases. In some embodiments, the variant panel may comprise variants identified through comprehensive genomic profiling (CGP), a next-generation sequencing (NGS) approach used to assess hundreds of genes (including relevant cancer biomarkers) in a single assay.

The variant in the variant panel may be of any size. The variant is associated with a reference sequence and a variant sequence; therefore, as long as the targeted variant is known a priori, the reference and variant sequences can be readily constructed. The variants in the variant panel can include, for example, one or more single nucleotide variants (SNVs), one or more multiple nucleotide variants (MNVs), a rearrangement junction, and/or one or more indels. The MNV may include two or more consecutive nucleotide variants and/or two or more single nucleotide variants spaced apart by nucleotide positions which comprise the same nucleotides as the reference sequence. In some embodiments, the variant panel includes one or more fusion variants or other rearrangement variants (e.g., an inversion or deletion event). The variants in the variant panel can include the locus of the variant and/or the variant relative to a reference sequence. Solely by way of example, a SNP variant can include the locus (e.g., a gene name and a base position within the gene, or a base position within a genome) and the variant (e.g., a C→G mutation).

The variant panel may include any number of variants that are associated with the disease, or example 1 or more, 2 or more, 5 or more, 10 or more, 25 or more, 50 or more, 100 or more, 500 or more, 1000 or more, 5000 or more, 10,000 or more, 20,000 or more, 50,000 or more, or 100,000 or more, or about 1 to about 10, about 10 to about 25, about 25 to about 100, about 100 to about 500, about 500 to about 1000, about 1000 to about 5000, about 5000 to about 10,000, about 10,000 to about 20,000, about 20,000 to about 50,000, or about 50,000 to about 100,000.

The variant panel or subject variant may include a rearrangement junction, in some embodiments. A rearrangement variant, such as an insertion, deletion, or inversion generates can generate two rearrangement junctions (or more in complex rearrangements) relative to a reference sequence. The junction may be detected using the methods described herein, for example by using a variant sequence that includes at least one of the junctions.

In some embodiments, the variant panel is a personalized variant panel generated for a particular subject. A sample can be acquired for the subject, and nucleic acid molecules (e.g., DNA, RNA, or both) within the sample are sequenced to generate sequencing reads. In some embodiments, the RNA molecules are reverse transcribed to form corresponding cDNA molecules. Variants can then be called from the generated sequencing reads using known variant calling methods.

The sample obtained from the subject may include nucleic acid molecules derived from the diseased tissue or a mixture of nucleic acid molecules derived from diseased tissue and nucleic acid molecules derived from healthy tissue (or two separate samples may be analyzed, using a first sample using nucleic acid molecules derived from diseased tissue and a second sample derived from healthy tissue). For example, the sample may include cell-free DNA (cfDNA) that includes circulating-tumor DNA (ctDNA, i.e., DNA naturally derived from a tumor tissue) and genomic cell-free DNA (i.e., cfDNA naturally derived from healthy tissue). The cfDNA can be sequenced and variants associated with the tumor called (either in reference to the genomic cell-free DNA, or in references to some other reference genome), and one or more of the called tumor variants can be included in the variant panel. In some embodiments, the sample may be derived from a tissue biopsy sample (e.g., a solid tissue sample or a hematological tissue sample) to obtain diseased tissue (e.g., a solid tumor biopsy sample or a hematological tumor biopsy sample) or healthy tissue. A nucleic acid sample can be derived from the tissue sample and can be used to generate sequencing reads.

In some embodiments, the variant panel is generated by calling variants between nucleic acid molecules obtained from a diseased tissue (e.g., a tumor tissue) and a healthy tissue. For example, the variants may be called using a matched normal, tumor sample.

In some embodiments the variant panel is generated by calling variants between nucleic acid molecules obtained from plasma (e.g., cfDNA) and nucleic acid molecules obtained from peripheral blood mononuclear cells (PBMCs).

In some embodiments, the sample used to acquire nucleic acid molecules may be blood, serum, saliva, tissue (for example, solid or hematological tissue), cerebral spinal fluid, amniotic fluid, peritoneal fluid, interstitial fluid, or embryonic tissue. In some embodiments, the tissue is a fresh tissue (i.e., not frozen or preserved). In some embodiments, the tissue is a frozen or reserved tissue (e.g., a formaldehyde-fixed paraffin embedded (FFPE) or paraformaldehyde-fixed paraffin-embedded (PFPE) tissue).

In some embodiments, the sample used to generate a personalized variant panel is obtained from the subject prior to the start of a disease therapy. In some embodiments, the sample used to generate the personalized variant panel is obtained from the subject after the start of the disease therapy.

The personalized variant panel can be generated for the subject having the disease using a personalized reference genome or sequence (e.g., a non-diseased genomic sequence of the subject) or a standard reference genome or sequence (e.g., a reference genome or reference sequence assembled from one or more other individuals, such as a standard or publicly available reference sequence, such as the Genome Reference Consortium human genome build 37 (GRCh37), or other suitable reference genome). Differences between the nucleic acid molecules derived from the diseased tissue can be compared to the reference, and variants identified.

In some embodiments, the variants in the variant panel include one or more variants known to be associated with the particular disease (such as a particular cancer) or with a population of subjects having the particular disease (such as a particular cancer). For example, the variant panel may include one or more variants curated from literature.

Variants in the variant panel are associated with a corresponding reference sequence and a corresponding variant sequence that includes the locus of the variant with left and right flanking regions (e.g., a 5′ flanking region and a 3′ flanking region). The left and right flanking regions of the variant locus provides context for the variant, and are the same for both the corresponding reference sequence and the corresponding variant sequence. Thus, the corresponding reference sequence and the corresponding variant sequence are identical except for the variant itself. The corresponding variant sequence includes the variant, and the corresponding reference sequence does not include the variant (e.g., it includes the reference or “wild-type” sequence at the location of the variant). In some embodiments, the flanking regions each include about 5 bases or more, about 10 bases or more, about 15 bases or more, about 20 bases or more, about 25 bases or more, about 30 bases or more, about 50 bases or more, about 75 bases or more, about 100 bases or more, about 150 bases or more, about 200 bases or more, about 250 bases or more, about 300 bases or more, about 400 bases or more, or about 500 bases or more. In some embodiments, the flanking regions each include between about 5 bases and about 5000 bases, such as about 5 to about 10 bases, about 10 to about 20 bases, about 20 to about 50 bases, about 50 to about 100 bases, about 100 to about 200 bases, about 200 to about 500 bases, about 500 to about 1000 bases, about 1000 bases to about 2500 bases, or about 2500 bases to about 5000 bases. In some embodiments, the left and right flanking regions have the same number of bases, and in some embodiments, the left and right flanking regions have a different number of bases.

The corresponding reference sequence and the corresponding variant sequence can be generated, for example, using the reference sequence used to identify the variant (which may be a personalized reference sequence or a standard reference sequence). To generate the corresponding variant sequence, the variant is selected and right and left flanking sequences are added to the variant using the reference sequence. To generate the corresponding reference sequence, the reference sequence is used using the same base locations as the corresponding variant sequence. Thus, in some embodiments, the corresponding reference sequence and corresponding variant sequence are identical except for the genetic variant.

The variant panel may be a list stored in a table or file (e.g., a variant call format (VCF) file or other suitable file format), which may be stored in a non-transitory computer-readable memory and can be accessed by one or more processors for executing one or more of the methods described herein. In some embodiments, the corresponding reference sequence and the corresponding variant sequence are stored in the same table or file as the variant panel, and in some embodiments, the corresponding reference sequence and the corresponding variant sequence are stored in a different table or file as the variant panel.

The variant panel may be a variant panel associate with a disease (such as cancer) or a personalized variant panel associated with a disease (such as cancer) in a subject. Exemplary diseases include, but are not limited to, B cell cancer, e.g., multiple myeloma, melanomas, breast cancer, lung cancer (such as non-small cell lung carcinoma or NSCLC), bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, adenocarcinomas, inflammatory myofibroblastic tumors, gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancers, essential thrombocythemia, agnogenic myeloid metaplasia, hypereosinophilic syndrome, systemic mastocytosis, familiar hypereosinophilia, chronic eosinophilic leukemia, neuroendocrine cancers, carcinoid tumors, and the like.

In some embodiments, the variants in the variant panel are not associated with a disease. For example, the variant panel may be used to support a previous call or a putative call. Whole genome sequencing and other sequencing methods may results in calls being made with low certainty. The methods described herein can be used to support (either positively or negatively) certain calls to provide higher sequence confidence.

In some embodiments, the variant panel comprises one or more variants (e.g., SNP, MNP, rearrangement junction or indel) within any of the following genes: ABCB1, ABCC2, ABCC4, ABCG2, ABL1, ABL2, AKT1, AKT2, AKT3, ALK, APC, AR, ARAF, ARFRP1, ARID1A, ATM, ATR, AURKA, AURKB, BCL2, BCL2A1, BCL2L1, BCL2L2, BCL6, BRAF, BRCA1, BRCA2, C1orf144, CARD11, CBL, CCND1, CCND2, CCND3, CCNE1, CDH1, CDH2, CDH20, CDH5, CDK4, CDK6, CDK8, CDKN2A, CDKN2B, CDKN2C, CEBPA, CHEK1, CHEK2, CRKL, CRLF2, CTNNB1, CYP1B1, CYP2C19, CYP2C8, CYP2D6, CYP3A4, CYP3A5, DNMT3A, DOT1L, DPYD, EGFR, EPHA3, EPHA5, EPHA6, EPHA7, EPHB1, EPHB4, EPHB6, ERBB2, ERBB3, ERBB4, ERCC2, ERG, ESR1, ESR2, ETV1, ETV4, ETV5, ETV6, EWSR1, EZH2, FANCA, FBXW7, FCGR3A, FGFR1, FGFR2, FGFR3, FGFR4, FLT1, FLT3, FLT4, FOXP4, GATA1, GNA11, GNAQ, GNAS, GPR124, GSTP1, GUCY1A2, HOXA3, HRAS, HSP90AA1, IDH1, IDH2, IGF1R, IGF2R, IKBKE, IKZF1, INHBA, IRS2, ITPA, JAK1, JAK2, JAK3, JUN, KDR, KIT, KRAS, LRP1B, LRP2, LTK, MAN1B1, MAP2K1, MAP2K2, MAP2K4, MCL1, MDM2, MDM4, MEN1, MET, MITF, MLH1, MLL, MPL, MRE11A, MSH2, MSH6, MTHFR, MTOR, MUTYH, MYC, MYCL1, MYCN, NF1, NF2, NKX2-1, NOTCH1, NPM1, NQO1, NRAS, NRP2, NTRK1, NTRK3, PAK3, PAX5, PDGFRA, PDGFRB, PIK3CA, PIK3R1, PKHD1, PLCG1, PRKDC, PTCH1, PTEN, PTPN11, PTPRD, RAF1, RARA, RB1, RET, RICTOR, RPTOR, RUNX1, SLC19A1, SLC22A2, SLCO1B3, SMAD2, SMAD3, SMAD4, SMARCA4, SMARCB1, SMO, SOD2, SOX10, SOX2, SRC, STK11, SULT1A1, TBX22, TET2, TGFBR2, TMPRSS2, TOP1, TP53, TPMT, TSC1, TSC2, TYMS, UGT1A1, UMPS, USP9X, VHL, and WT1.

In some embodiments the variant is a mutation, for example a mutation associated with a tumor. In some embodiments, the variant is a somatic mutation. In some embodiments, the variant is a germline mutation.

### Labeling Sequencing Reads

Sequencing reads can be labeled as including a genetic variant or as not including a genetic variant. In some embodiments, a sequencing read can be labeled as inconclusive, which indicates that the sequencing read cannot be labeled as having the variant or as not having the variant, as discussed in more detail below. Sequencing reads can be mapped to a location within a reference sequence, and the mapped location is used to select a genetic variant from the variant panel associated with the locus. Once the variant and the sequencing read are associated, the sequencing read is alleged with a reference sequence (i.e. a corresponding sequence that does not include the variant) to generate a reference match score, and a variant sequence (i.e., a corresponding sequence that includes the variant) to generate a variant match score. The sequencing read can be labeled as having the variant if the reference match score and the variant match score indicate that the sequencing read more closely matches with the variant sequence than the reference sequence, or as not having the variant if the reference match score and the variant match score indicate that the sequencing read more closely matches with the reference sequence. In some embodiments, the sequencing read is labeled as an inconclusive read if the reference match score and the variant match score are equal.

In some embodiments, a method of detecting the presence or absence of a variant or determining a variant allele frequency in a test sample from a subject, comprising (a) selecting a genetic variant at a variant locus from a variant panel; (b) obtaining one or more sequencing reads associated with the test sample that overlap the variant locus; (c) generating a reference match score for each of the one or more sequencing reads by aligning each sequencing read to a corresponding reference sequence, wherein the corresponding reference sequence does not comprise the genetic variant; (d) generating a variant match score for each of the one or more sequencing reads by aligning each sequencing read to a corresponding variant sequence, wherein the corresponding variant sequence comprises the genetic variant; and (e) labeling each of the one or more sequencing reads as either having the genetic variant, not having the genetic variant, or a being an inconclusive read based on the reference match score and the variant match score; wherein: a sequencing read is labeled as having the genetic variant if the reference match score and the variant match score indicate that the sequencing read more closely matches the variant sequence than the reference sequence; a sequencing read is labeled as not having the genetic variant if the reference match score and the variant match score indicate that the sequencing read more closely matches the reference sequence than the variant sequence; and a sequencing read is labeled as an inconclusive read if the reference match score and the variant match score are equal.

Sequencing reads can be aligned to a reference sequence to determine a location of the sequencing read within a reference genome. The alignment can be used to generate a sequence alignment map file (e.g., a SAM or BAM file), which includes a mapping position for the read. The variant panel can then be accessed to select a genetic variant, and one or more sequencing reads that overlap the locus of the variant can be obtained (for example, by accessing the sequencing alignment map file). The overlap may be at one or more base positions of the variant (for example, if the variant is a multi-base variant). In some embodiments, sequencing reads that overlap the same single base (e.g., the first base) of the variant are used. A corresponding reference sequence and a corresponding variant sequence are also selected, wherein the corresponding reference sequence and the corresponding variant sequence are associated with the selected variant.

The reference match score for any given sequencing read is generated by aligning the sequencing read to the corresponding reference sequence, and the variant match score is generated by aligning the sequencing read to the corresponding variant sequence. The reference match score and the variant match score are generated using the same alignment algorithm so that the reference match score and the variant match score are comparable. The match score provides a value that indicates how closely matched the query sequence (e.g., the sequencing read) is to the corresponding variant sequence or corresponding reference sequence. Exemplary alignment algorithms include the Smith-Waterman Algorithm (SWA) (e.g., a Striped Smith-Waterman Algorithm) or the Needleman-Wunsch Algorithm (NWA). In some embodiments, the reference match score and the variant match score are generated using the Smith-Waterman Algorithm. In some embodiments, the reference match score and the variant match score are generated using the Striped Smith-Waterman Algorithm. In some embodiments, the reference match score and the variant match score are generated using the Needleman-Wunsch algorithm.

The sequencing reads are labeled by comparing the variant match score and the reference match score. For example, the sequencing read is labeled as having the genetic variant if the reference match score and the variant match score indicate that the sequencing read more closely matches the variant sequence than the reference sequence. The sequencing read is labeled as not having the genetic variant if the reference match score and the variant match score indicate that the sequencing read more closely matches the reference sequence than the variant sequence. In some instances, the reference match score and the variant match score are equal, in which case the sequencing read may be labeled as an inconclusive read. In some embodiments, a sequencing read labeled as an inconclusive read is excluded from further analysis.

The sequencing reads can be obtained by sequencing nucleic acid molecules in a test sample derived from a subject. In some embodiments, the test sample is the same type of sample as the test sample used to determine the genetic variants in a personalized variant panel. Exemplary test samples include, but are not limited to blood, serum, saliva, tissue (for example, solid or hematological tissue), cerebral spinal fluid, amniotic fluid, peritoneal fluid, interstitial fluid, or embryonic tissue. In some embodiments, the tissue is a fresh tissue (i.e., not frozen or preserved). In some embodiments, the tissue is a frozen or reserved tissue (e.g., a formaldehyde-fixed paraffin embedded (FFPE) or paraformaldehyde-fixed paraffin-embedded (PFPE) tissue).

In some embodiments, the test sample is derived from a liquid biopsy sample (e.g., plasma, peripheral blood, etc.). The liquid biopsy may be divided into two or more matched samples or sample components. For example, the sample may include a plasma component (which can include cfDNA) and a peripheral blood mononuclear cell (PBMC) component. The individual components may be analyzed separately to determine differences between the genetic profile of each component. This can be used, for example, to identify somatic mutations or clonal hematopoiesis.

In some embodiments, the sample is derived from a solid tissue biopsy sample. The tissue biopsy may include cancerous cells, non-cancerous (e.g., healthy) cells, or a mixture thereof. In some embodiments, the tissue biopsy sample is a fresh tissue (i.e., not frozen or preserved). In some embodiments, the tissue is a frozen or reserved tissue (e.g., a formaldehyde-fixed paraffin embedded (FFPE) or paraformaldehyde-fixed paraffin-embedded (PFPE) tissue).

The nucleic acid molecules in the test sample may be DNA, RNA, or a mixture thereof. In some embodiments, the RNA molecules are reverse transcribed to form corresponding cDNA molecules. The test sample obtained from the subject may include nucleic acid molecules derived from the diseased tissue or a mixture of nucleic acid molecules derived from diseased tissue and nucleic acid molecules derived from healthy tissue. For example, sample may include cell-free DNA (cfDNA) that included circulating-tumor DNA (ctDNA, i.e., DNA naturally derived from a tumor tissue) and genomic cell-free DNA (i.e., cfDNA naturally derived from healthy tissue). In some embodiments, the sample may be derived from a tissue biopsy sample (e.g., a solid tissue sample or a hematological tissue sample) to obtain diseased tissue (e.g., a solid tumor biopsy sample or a hematological tumor biopsy sample) or healthy tissue. A nucleic acid sample can be derived from the tissue sample and can be used to generate sequencing reads.

The described method for labeling sequencing reads can be repeated for any number of variants using different genetic variants at different loci selected from the genetic variant panel.

In some embodiments, the labeled sequencing reads are used to call the presence of the genetic variant in the sample from the subject. For example, if one or more sequencing reads (or one or more unique sequencing reads) are labeled as having the genetic variant, the presence of the genetic variant may be called. The threshold set for calling the presence of the genetic variant can be set as desired, depending on the desired confidence for making the call. For example, in some embodiments, the threshold for calling the presence of the genetic variant can be called as 1, 2, 3, 4, 5, 6, 7, 8, 9, 10 or more sequencing reads (or unique sequencing reads) labeled as having the genetic variant, wherein the presence of the genetic variant is called if the number of sequencing reads (or unique sequencing reads) labeled as having the genetic variant meets or is higher than the threshold.

In some embodiments, the labeled sequencing reads are used to determine the variant allele frequency for the variant in the sample. A variant allele frequency (Fᵢ) at locus i for the test sample can be determined using the number of sequencing reads labeled as having the variant (Vᵢ) and the number of sequencing reads as not having the variant (Rᵢ) according to

$${F}_{i} = \frac{{V}_{i}}{{V}_{i} + {R}_{i}} .$$

The methods described herein may be used to determine the variant allele frequency in a sample, two or more different tissues or samples, or two or more different components of the same sample. For example, a blood draw may be divided into plasma (which contains cfDNA) and peripheral blood mononuclear cells (PBMCs). A first variant allele frequency may be determined for the first sample or the first sample component (e.g., the plasma), and a second variant allele frequency may be determined for the second sample or second sample component (e.g., the PBMCs). The difference in variant allele frequency between, for example, nucleic acid molecules from plasma and nucleic acid molecules from PBMC is useful for subjects with clonal hematopoiesis or clonal hematopoiesis of indeterminate potential (CHIP).

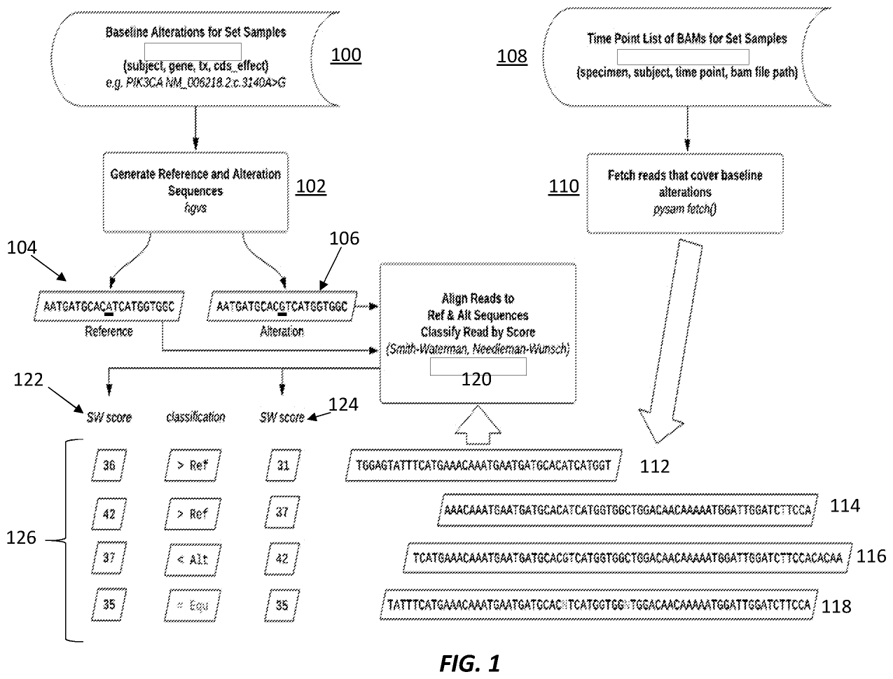

FIG. 1 shows an exemplary embodiment of a method for labeling sequencing reads. At step 100, the genetic variant panel (i.e., the baseline alternations) is generated by sequencing an initial sample obtained from the subject. The genetic variant panel may include information about each genetic variant in the panel, for example a subject identifier, the gene containing the variant, the locus of the variant, and/or the variant change (relative to reference). At corresponding sequence generation module 102, corresponding reference sequence 104 and corresponding variant sequencing read 106 are generated using a variant from the variant panel and a reference sequence used to provide context for the variant. The corresponding reference sequence 104 and the corresponding variant sequencing read 106 are identical except for at the variant locus, wherein an A→G SNP is present (indicated by underline). Sequencing reads obtained by sequencing a second test sample acquired from a subject are aligned to a reference sequence, and the mapped sequencing reads are included in an alignment map file 108. The alignment map file 108 includes the sequences from the sequencing reads, along with the locus information for the sequencing reads. Optionally, the alignment map file 108 may include additional information, such as information about the subject, the time point at which the sample was acquired, and/or other sample information. A variant is selected from the variant table, and sequencing reads that overlap the locus of the variant read are retrieved from the alignment map file 108 at sequencing read retrieving module 110. In the example shown in FIG. 1, sequencing reads 112, 114, 116, and 118 represent the sequencing reads that overlap the locus of the selected variant. At alignment module 120, the sequencing reads 112, 114, 116, and 118 are each aligned with the corresponding reference sequence 104 to generate a reference match score 122, and the corresponding variant sequencing read 106 to generate a variant match score 124. The reference match score 122 and the variant match score 124 can be generated using an alignment algorithm, such as a Smith-Waterman algorithm or a Needleman-Wunsch algorithm. At classification module 126, for each sequencing read, the reference match score and the variant match score are compared to label the sequencing read as a having the variant, not having the variant, or being an inconclusive read. In the example illustrated in FIG. 1, sequencing reads 112 and 114 are labeled not having the variant because the reference match score is greater than the variant match score for each read. Sequencing read 116 is labeled as having the variant because the variant match score is greater than the reference match score. Sequencing read 118 is labeled as an inconclusive read because the variant match score equals the reference match score.

Embodiments in accordance with this disclosure can provide an exemplary method for determining a variant frequency in a test sample from a subject. At an initial step, a genetic variant at a variant locus is selected from a variant panel. In some embodiments, the variant panel is a personalized variant panel. At another step, sequencing reads that overlap the variant locus and are associated with the test sample are obtained. A reference match score for each sequencing read is obtained by aligning the sequencing reads to a corresponding reference sequence at another step, and a variant match score for each sequencing read is generated by aligning the sequencing reads to a corresponding variant sequence at another step. Using the reference match score and the variant match score, the sequencing reads are labeled as having the variant, not having the variant, or as an inconclusive read at another step. At another step, the genetic variant frequency is determined using the number of sequencing reads labeled as having the variant and the number sequencing reads labeled as not having the variant.

In some embodiments, the method includes generating or updating a report (such as a printed report or an electronic medical record). The report can include one or more of a call for the presence or absence of the genetic variant, a call for the variant allele frequency, and/or a disease status. The report can also include identifying information for the subject (e.g., name, identification number, etc.). The report may be stored or transmitted to another person or entity, for example, the subject or a healthcare provider (e.g., a doctor, nurse, caretaker, hospital, clinic, etc.).

### Disease Status and Monitoring Disease Progression or Recurrence

A disease status can be determined using the variant frequency in the test sample at one or more variant loci. In some embodiments, an increase in variant frequency indicates an increase in the severity of the disease. In some embodiments, sequencing reads labeled as having the genetic variant are attributed to disease tissue. In some embodiments, sequencing reads labeled as not having the genetic variant are attributed to the non-diseased tissue. In some embodiments, sequencing reads labeled as having the genetic variant are attributed to disease tissue, and sequencing reads labeled as not having the genetic variant are attributed to the non-diseased tissue. In some embodiments, sequencing reads labeled as having the genetic variant are attributed to a first diseased tissue, and sequencing reads labeled as not having the genetic variant are attributed to a second diseased tissue and/or a non-diseased tissue.

In some embodiments, one or more genetic variants are used to characterize the disease or cancer. For example, the presence of one or more genetic variants may be used to trace the original source of the disease (e.g., a primary cancer). In some embodiments, the detection of one or more genetic variants can be used to characterize a therapy-resistant cancer or cancer as being particularly susceptible to a particular treatment. A variant panel used to characterize the disease may be based on known variants, for example those curated from literature.

In some embodiments, the disease status is determined on a per variant status. In some embodiments, the disease status is determined using a plurality of variants from the variant panel. For example, in some embodiments, a disease status (DS) can be determined using a total number of sequencing reads (or a total number of unique sequencing reads) determined as having a variant (V_{T}) and a total number of sequencing reads (or a total number of unique sequencing reads) determined as not having a variant (R_{T}), according to

$$\frac{{V}_{T}}{{V}_{T} + {R}_{T}} .$$

The disease status may be determined for a plurality of genetic variants, for example as a summary statistic. In some embodiments, variants associated with germline mutations are excluded from the determination of the disease status. In some embodiments, variants associated with clonal hematopoiesis are excluded from determination of the disease status. In some embodiments, the disease status is qualitatively assessed, for example by identifying the subject has having cancer, having a recurrence of the cancer, having a cancer that is resistant to a particular treatment modality, or having a cancer that can be treated with a particular treatment modality. In some embodiments, the disease status is quantitatively assessed (e.g., a determined tumor fraction of cfDNA, or a maximum somatic allele fraction of cfDNA).

Disease progression can be monitored by determining a disease status at two or more time points. The disease status can be indicated by the variant frequency in the test sample. For example, a first test sample may be obtained from the subject at a first time point, and a second test sample may be obtained from the subject at a second time point. In some embodiments, the first test sample is used to generate the variant panel and is used to determine the disease status at the first time point, and the second test sample uses the generated variant panel to determine the disease status at the second time point.

The subject may receive treatment for the disease between the first test sample and the second test sample (i.e., an intervening treatment). Thus, by monitoring the disease progression, it can be determined whether the treatment therapy is effective in treating the disease. The treatment therapy may further be adjusted depending on the disease progression. For example, a therapeutic dose may be increased or an alternative treatment therapy used if the disease worsens or fails to improve.

The time period between the first time point and the second time point can be as frequent as desired to effectively monitor the subject. In some embodiments, the first time point and the second time point is about 1 week or more, about 2 weeks or more, about 4 weeks or more, about 8 weeks or more, about 12 weeks or more, about 16 weeks or more, about 6 months or more, about 1 year or more, or about 2 years or more.

In some embodiments, monitoring the subject for disease progression includes monitoring the subject for disease recurrence. For example, a subject deemed to be in remission may have a minimal amount of residual disease that has some recurrence risk. A test sample of the subject may be occasionally obtained and a disease status determined to see if the disease has recurred. If the disease status has recurred, then the subject can be treated for the recurring disease.

In some embodiments, a method of monitoring disease progression includes sequencing nucleic acid molecules in a first test sample acquired from a subject with a disease to generate first sequencing reads; generating a personalized variant panel for the subject; sequencing nucleic acid molecules in a second test sample acquired from the subject at a later time point than the first test sample to generate second sequencing reads; and labeling the second sequencing reads. The sequencing reads may be labeled, for example, by selecting a genetic variant at a variant locus from the personalized variant panel; (b) obtaining one or more sequencing reads associated with the test sample that overlap the variant locus; (c) generating a reference match score for each of the one or more sequencing reads by aligning each sequencing read to a corresponding reference sequence, wherein the corresponding reference sequence does not comprise the genetic variant; (d) generating a variant match score for each of the one or more sequencing reads by aligning each sequencing read to a corresponding variant sequence, wherein the corresponding variant sequence comprises the genetic variant; and (e) labeling each of the one or more sequencing reads as either having the genetic variant, not having the genetic variant, or a being an inconclusive read, based on the reference match score and the variant match score; wherein: a sequencing read is labeled as having the genetic variant if the reference match score and the variant match score indicate that the sequencing read more closely matches the corresponding variant sequence than the corresponding reference sequence; a sequencing read is labeled as not having the genetic variant if the reference match score and the variant match score indicate that the sequencing read more closely matches the corresponding reference sequence than the corresponding variant sequence; and a sequencing read is labeled as an inconclusive read if the reference match score and the variant match score are equal.

Embodiments in accordance with the present disclosure can provide methods for monitoring disease progression. The method includes, at an initial step, sequencing nucleic acid molecules in a first test sample obtained from a subject with a disease to generate first sequencing reads. From the first sequencing reads, a personalized variant panel is generated for the subject. At another step, a disease status for the subject can be determined, which is indicative of the disease severity for the subject. The disease status may be represented, for example, by a variant frequency determined for the subject. After a period of time, a second test sample can be obtained from the subject. At another step, nucleic acid molecules in the second test sample are sequenced. At another step, a genetic variant at a variant locus is selected from the personalized variant panel. At another step, sequencing reads that overlap the variant locus and are associated with the test sample are obtained. A reference match score for each sequencing read is obtained by aligning the sequencing reads to a corresponding reference sequence, and a variant match score for each sequencing read is generated by aligning the sequencing reads to a corresponding variant sequence at another step. Using the reference match score and the variant match score, the sequencing reads are labeled as having the variant, not having the variant, or as an inconclusive read at another step. At another step, the genetic variant frequency is determined using the number of sequencing reads labeled as having the variant and the number sequencing reads labeled as not having the variant. Using the determined variant frequency, a disease status for the subject can be determined indicating the severity of the disease that the time the second sample is obtained from the subject.

In some embodiments, the monitored disease is a cancer. For example, in some embodiments, the disease is B cell cancer, e.g., multiple myeloma, melanomas, breast cancer, lung cancer (such as non-small cell lung carcinoma or NSCLC), bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, adenocarcinomas, inflammatory myofibroblastic tumors, gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancers, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.

In some embodiments, the methods described herein are used to identify a viral or bacterial strain. Bacteria and viruses can mutate, and clearly distinguishing between particular strain types can be particularly important for treating an infected subject. For example, it is important to know whether a strain of Staphylococcus aureus infecting a subject is resistant to methicillin and/or vancomycin. Antibiotic or other drug resistant bacteria and viruses have a genomic signature, and the methods described herein can be used to quickly characterize different strains.

### Disease Treatments

The methods described herein may be used when treating a subject with a disease. As discussed above, the method may include monitoring disease progression, such as cancer progression in the subject. Monitoring disease progression allows a clinician to provide better treatment decisions, and can be used to screen for disease (e.g., cancer) recurrence or metastasis.

A first test sample can be acquired from a subject having the disease, and nucleic acid molecules from the test sample can be sequenced to generate first sequencing reads, which are used to generate a personalized variant panel for the subject. A disease therapy is then administered to the subject and, after a period of time, a second test sample is acquired from the subject at a second time point. Nucleic acid molecules from the second test sample can be sequence to generate second sequencing reads, and the second sequencing reads can be labeled using the methods described herein. For example, the second sequencing reads may be labeled by selecting a genetic variant at a variant locus from the personalized variant panel; (b) obtaining one or more sequencing reads associated with the test sample that overlap the variant locus; (c) generating a reference match score for each of the one or more sequencing reads by aligning each sequencing read to a corresponding reference sequence, wherein the corresponding reference sequence does not comprise the genetic variant; (d) generating a variant match score for each of the one or more sequencing reads by aligning each sequencing read to a corresponding variant sequence, wherein the corresponding variant sequence comprises the genetic variant; and (e) labeling each of the one or more sequencing reads as either having the genetic variant, not having the genetic variant, or a being an inconclusive read, based on the reference match score and the variant match score; wherein: a sequencing read is labeled as having the genetic variant if the reference match score and the variant match score indicate that the sequencing read more closely matches the corresponding variant sequence than the corresponding reference sequence; a sequencing read is labeled as not having the genetic variant if the reference match score and the variant match score indicate that the sequencing read more closely matches the corresponding reference sequence than the corresponding variant sequence; and a sequencing read is labeled as an inconclusive read if the reference match score and the variant match score are equal. A first disease status can be determined using the first sequencing reads, and a second disease status can be determined using the labeled second sequencing reads. Disease progression can be determined by comparing the first disease status and the second disease status. The disease therapy administered to the subject can be adjusted based on the disease progression, and the adjusted disease therapy can then be administered to the subject.

In an exemplary embodiments, a method of treating a subject with a disease (such as cancer) includes: acquiring a first test sample from the subject; sequencing nucleic acid molecules in a first test sample to generate first sequencing reads; determining a first disease status using the first sequencing reads; generating a personalized variant panel for the subject; administering a disease therapy to the subject; acquiring a second test sample from the subject after the disease therapy has been administered to the subject; sequencing nucleic acid molecules in the second test sample to generate second sequencing reads; labeling the second sequencing reads by (a) selecting a genetic variant at a variant locus from a variant panel; (b) obtaining one or more sequencing reads associated with the test sample that overlaps the variant locus; (c) generating a reference match score for each of the one or more sequencing reads by aligning each sequencing read to a corresponding reference sequence, wherein the corresponding reference sequence does not comprise the genetic variant; (d) generating a variant match score for each of the one or more sequencing reads by aligning each sequencing read to a corresponding variant sequence, wherein the corresponding variant sequence comprises the genetic variant; and (e) labeling each of the one or more sequencing reads as either having the genetic variant, not having the genetic variant, or a being an inconclusive read, based on the reference match score and the variant match score; wherein: a sequencing read is labeled as having the genetic variant if the reference match score and the variant match score indicate that the sequencing read more closely matches the corresponding variant sequence than the corresponding reference sequence; a sequencing read is labeled as not having the genetic variant if the reference match score and the variant match score indicate that the sequencing read more closely matches the corresponding reference sequence than the corresponding variant sequence; and a sequencing read is labeled as an inconclusive read if the reference match score and the variant match score are equal; determining a second disease status using the labeled second sequencing reads; determining disease progression by comparing the first disease status and the second disease status; adjusting the disease therapy administered to subject based on the disease progression; and administering the adjusted disease therapy to the subject.

In some embodiments, the disease therapy (such as cancer therapy for treating a cancer) comprises surgery (for example, an excision surgery to remove one or more cancers). In some embodiments, the disease therapy comprises a radiation therapy (such as external beam radiation therapy, stereotactic radiation, intensity-modulated radiation therapy, volumetric modulated arc therapy, particle therapy (such as proton therapy), auger therapy, brachytherapy, or systemic radioisotope therapy). In some embodiments, the disease therapy comprises the administration of one or more chemical agents, such as one or more chemotherapeutic agents for the treatment of cancer. Exemplary chemotherapeutic agents include, but are not limited to, anthracyclines (such as daunorubicin, epirubicin, idarubicin, mitoxantrone, valrubicin) alkylating or alkylating-like agents (such as carboplatin, carmustine, cisplatin, cyclophosphamide, melphalan, procarbazine, or thiotepa), or taxanes (such as paclitaxel, docetaxel, or taxotere).

In some embodiments, the therapy is an immunotherapy. In some embodiments, the therapy is an immune checkpoint inhibitor.

In some embodiment, the disease therapy is a targeted therapy. Exemplary targeted therapies include tyrosine-kinase inhibitors (e.g., imatinib, gefitinib, erlotinib, sorafenib, sunitnib, dasatinib, lapatinib, nilotinib, bortezomib, JAK inibitors (e.g., tofacitinib), ALK inibitors (e.g., crizotinib), BCL-2 inhibitors (e.g., obatoclax, navitoclax, gossypol), PARP inibitiors (e.g., iniparib, olaparib), PI3K inibhtors (e.g., perifosine), apatinib, BRAF inhibitors (e.g., vemurafenib, dabrafenib, LGX818), MEK inhibitors (e.g., trametinib, MEK162), CDK inhibitors, Hsp90 inhibitors, or salinomycin), serine/threonine kinase inhibitors (e.g., temsirolimus, everolimus, vemurafenib, trametinib, or dabrafenib), or a monocolonal antibody (e.g., pembrolizumab, rituximab, trastuzumab, alemtuzumab, cetuximab, panitumumab, or bevacizumab).

In some embodiments, the therapeutic agent administered to the subject is selected based on calling a genetic variant in the sample using the methods described herein. For example, the detection of specific biomarkers using the methods described herein can be used as a basis for selecting a particular therapy modality. Exemplary personalized therapy selections for a given identified mutations are listed in Table 1.

TABLE 1

| Tumor Type | Genetic Variant Call | Therapy |
|---|---|---|
| Non-small cell lung cancer | EGFR exon 19 deletions or | Afatinib, gefitinib, |
| (NSCLC) | EGFR exon 21 L858R | osimertinib, or erlotinib |
| | alterations | |
| | EGFR exon 20 T790M | Osimertinib |
| | alterations | |
| | ALK rearrangement | Alectinib, crizotinib, or |
| | | ceritinib |
| | BRAF V600E | Dabrafenib and trametinib |
| | | combination |
| | MET single nucleotide | Capmatinib |
| | variant or indel that results | |
| | in MET exon 14 skipping | |
| Melanoma | BRAF V600E | Dabrafenib, or vemurabenib |
| | BRAF V600E and V600K | Trametinib; or cobimetinib |
| | | in combination with |
| | | vemurafenib |
| Breast Cancer | PIK3CA C420R, E542K, | Alpelisib |
| | E545A, E545D (1635G > T), | |
| | E545G, E545K, Q546, | |
| | Q546R, H1047L, H1047R, | |
| | or H1047 | |
| Colorectal cancer | KRAS wild-type (i.e., | Cetuxmab |
| | absence of variant) in codon | |
| | 12 and 13 | |
| | KRAS wild-type (i.e., | Panitumumab |
| | absence of variant) in codon | |
| | 2, 3, and 4; and/or NRAS | |
| | wild type (i.e., absence of | |
| | variant) in codon 2, 3, and 4 | |
| Ovarian cancer | BRCA1/2 variant | Olaparib or rucaparib |
| Cholangiocarcinoma | FGFR2 fusion or | Pemigatinib |
| | rearrangement | |
| Prostate cancer | Variant in a Homologous | Olaparib |
| | Recombination Repair | |
| | (HRR) gene variant (e.g., | |
| | one or more of BRCA1, | |
| | BRCA2, ATM, BARD1, | |
| | BRIP1, CDK12, CHEK1, | |
| | CHEK2, FANCL, PALB2, | |
| | RAD51B, RAD51C, | |
| | RAD51D, or RAD54L) | |

In some embodiments, the treated disease is a cancer. For example, in some embodiments, the disease is B cell cancer, e.g., multiple myeloma, melanomas, breast cancer, lung cancer (such as non-small cell lung carcinoma or NSCLC), bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, adenocarcinomas, inflammatory myofibroblastic tumors, gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancers, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.

### Computer Systems and Methods

The methods described herein may be implemented using one or more computer systems. Such computer systems can include one or more programs configured to execute one or more processors for the computer system to perform such methods. One or more steps of the computer-implemented methods may be performed automatically.

In some embodiments, the computer-implemented method for detecting the presence of a genetic variant and/or determining a variant allele frequency in a test sample from a subject, or labeling sequencing reads associated with a test sample from a subject, includes (a) selecting, using one or more processors, a genetic variant at a variant locus from a variant panel stored in a memory; (b) receiving, at the one or more processors, one or more sequencing reads stored in the memory, wherein the sequencing reads are associated with the test sample that overlaps the variant locus; (c) generating, using the one or more processors, a reference match score for each of the one or more sequencing reads by aligning each sequencing read to a corresponding reference sequence retrieved from the memory, wherein the corresponding reference sequence does not comprise the genetic variant; (d) generating, using the one or more processors, a variant match score for each of the one or more sequencing reads by aligning each sequencing read to a corresponding variant sequence retrieved from the memory, wherein the corresponding variant sequence comprises the genetic variant; and (e) labeling, using the one or more processors, each of the one or more sequencing reads as either having the genetic variant, not having the genetic variant, or a being an inconclusive read, based on the reference match score and the variant match score; wherein: a sequencing read is labeled as having the genetic variant if the reference match score and the variant match score indicate that the sequencing read more closely matches the corresponding variant sequence than the corresponding reference sequence; a sequencing read is labeled as not having the genetic variant if the reference match score and the variant match score indicate that the sequencing read more closely matches the corresponding reference sequence than the corresponding variant sequence; and a sequencing read is labeled as an inconclusive read if the reference match score and the variant match score are equal.

In some embodiments of the computer-implemented method, the method further includes generating the corresponding reference sequence and/or the corresponding variant sequence. In some embodiments, the corresponding reference sequence and the corresponding variant sequence are identical except for the genetic variant.

In some embodiments of the computer-implemented method, the one or more sequencing reads comprises a plurality of sequencing reads overlapping the variant locus, and the method further comprises determining a number of sequencing reads from the plurality of sequencing reads having the genetic variant or a number of sequencing reads from the plurality of sequencing reads not having the genetic variant. In some embodiments, the method further comprises determining a variant frequency for the genetic variant using the number of sequencing reads having the genetic variant and the number of sequencing reads not having the genetic variant.

In some embodiments of the computer-implemented method, the method includes labeling one or more sequencing reads associated with the test sample for a plurality of genetic variants at different variant loci selected from the variant panel.

In some embodiments of the computer-implemented method, the method includes determining a disease status for the subject. For example, the disease status may be a value proportional to a percentage of circulating-tumor DNA (ctDNA) compared to total cell-free DNA (cfDNA) in the test sample.

In some embodiments, the reference match score and the variant match score are determined using a sequence alignment algorithm. In some embodiments, the reference match score and the variant match score are determined using a Smith-Waterman alignment algorithm. In some embodiments, the reference match score and the variant match score are determined using a Needleman-Wunsch alignment algorithm.

Embodiments in accordance with the present disclosure can provide a computer-implemented method for determining a variant frequency in a test sample from a subject. An initial step 402 includes selecting, using one or more processors, a genetic variant at a variant locus from a variant panel stored in a memory. In some embodiments, this step includes receiving genetic variant and variant locus information for one or more variants from the variant panel stored in the memory. For example, the processor may accesses the memory to retrieve the genetic variant and variant locus information, which can be listed in a table or file stored on the memory. Selection is made from the variant panel through any suitable process (e.g., randomly, sequentially, using a prioritization rank). In some embodiments, the computer-implemented method is repeated until a desired number (or all) of the variants in the variant panel are analyzed.

Another step can include receiving, at the one or more processors, one or more sequencing reads stored in the memory, wherein the sequencing reads are associated with the test sample that overlaps the variant locus. For example, the processor may access the memory to retrieve the one or more sequencing reads that overlap the variant locus. The memory may store a table or file containing sequencing reads (e.g., a BAM or SAM file), which includes the read and the read locus. Those sequencing reads in the table or file that overlap with the locus of the selected variant can then be selected and received at the one or more processors.

Another step can include generating, using the one or more processors, a reference match score for each of the one or more sequencing reads by aligning each sequencing read to a corresponding reference sequence retrieved from the memory, wherein the corresponding reference sequence does not comprise the genetic variant. In some embodiments, this step includes receiving a reference sequence corresponding to the selected variant (i.e., a corresponding reference sequence). For example, the corresponding reference sequence may be stored in a table or file in the memory. In some embodiments, the table or file storing the corresponding reference sequence is the same table or file storing information about the selected variant or the variant panel. In some embodiments, the table or file storing the corresponding reference sequence is a different table or file from the table or file storing information about the selected variant or the variant panel. Each sequencing read corresponding to the selected variant and received at the one or more processors is aligned to the corresponding reference sequence using an alignment module. The alignment module implements an alignment algorithm (such as a Smith-Waterman alignment algorithm or a Needleman-Wunsch alignment algorithm) to generate the reference match score. In some embodiments, the reference match score is stored in the memory, for example by automatically updating the table or file storing the sequencing reads or by automatically generating a new table or file containing the reference match score and the associate read or a read identifier.

Another step can include generating, using the one or more processors, a variant match score for each of the one or more sequencing reads by aligning each sequencing read to a corresponding variant sequence retrieved from the memory, wherein the corresponding variant sequence comprises the genetic variant. In some embodiments, this step includes receiving a variant sequence corresponding to the selected variant (i.e., a corresponding variant sequence). For example, the corresponding variant sequence may be stored in a table or file in the memory (which may be the same file or table as the table or file storing the corresponding reference sequence, or a different file). In some embodiments, the table or file storing the corresponding variant sequence is the same table or file storing information about the selected variant or the variant panel. In some embodiments, the table or file storing the corresponding variant sequence is a different table or file from the table or file storing information about the selected variant or the variant panel. Each sequencing read corresponding to the selected variant and received at the one or more processors is aligned to the corresponding variant sequence using an alignment module. The alignment module implements an alignment algorithm (generally the same alignment algorithm used to align the sequencing read with the reference alignment module) to generate the variant match score. In some embodiments, the variant match score is stored in the memory, for example by automatically updating the table or file storing the sequencing reads or by automatically generating a new table or file containing the reference match score and the associate read or a read identifier. In some embodiments, a table or file is automatically generated that includes both the reference match score and the variant match score.

Another step can include labeling, using the one or more processors, each of the one or more sequencing reads as either having the genetic variant, not having the genetic variant, or a being an inconclusive read, based on the reference match score and the variant match score; wherein: a sequencing read is labeled as having the genetic variant if the reference match score and the variant match score indicate that the sequencing read more closely matches the corresponding variant sequence than the corresponding reference sequence; a sequencing read is labeled as not having the genetic variant if the reference match score and the variant match score indicate that the sequencing read more closely matches the corresponding reference sequence than the corresponding variant sequence; and a sequencing read is labeled as an inconclusive read if the reference match score and the variant match score are equal. In some embodiments, the step of labeling, using the one or more processors, each of the one or more sequencing reads as either having the genetic variant, not having the genetic variant, or a being an inconclusive read, is based on the reference match score and the variant match score is implemented by a labeling module. The labeling module can compare the variant match score and the reference match score. A sequencing read is labeled as having the genetic variant if the reference match score and the variant match score indicate that the sequencing read more closely matches the corresponding variant sequence than the corresponding reference sequence. The sequencing read is labeled as not having the genetic variant if the reference match score and the variant match score indicate that the sequencing read more closely matches the corresponding reference sequence than the corresponding variant sequence. Further, in some embodiments, the sequencing read is labeled as an inconclusive read if the reference match score and the variant match score are equal. In some embodiments, the label associated with the sequencing read is automatically stored in the memory. For example, in some embodiments, the one or more processors automatically accesses a table or file stored on the memory and updates the file to include the labels for the sequencing reads. In some embodiments, the one or more processors automatically generates a table or file and stores it on the memory, which includes the labels for the sequencing reads.

Another step can include determining, using the one or more processors, a genetic variant frequency using a number of sequencing reads having the variant and a number of sequencing reads not having the variant. In some embodiments, the one or more processors automatically generates or updates a table or file in the memory to record the genetic variant frequency.

The computer-implemented method for detecting a genetic variant or determining an allele frequency for the genetic variant in a test sample from a subject can include the use of an electronic system that includes one or more processors and a memory storing a reference sequence and a variant sequence pair. The reference sequence and the variant sequence pair correspond with a genetic variant being queried by the method, which may be selected, using the one or more processors, from a variant panel stored on the memory. The one or more processors can receive one or more sequencing reads from the test sample, wherein the sequencing reads overlap the genetic locus of the queried genetic variant. The one or more processors can also receive the reference sequence from the memory and generate a reference match score for each of the one or more sequencing reads by aligning each sequencing read to the corresponding reference sequence. Further, the one or more processors can receive the variant sequence from the memory and generate a variant match score for each of the one or more sequencing reads by aligning each sequencing rad to the corresponding variant sequence. Based on the reference match score and the variant match score, the sequencing reads can be labeled as having the genetic variant or not having the genetic variant. In some embodiments, a sequencing read can be labeled as inconclusive, which indicates that the sequencing read cannot be labeled as having the variant or as not having the variant, e.g., the reference match score and the variant match score are equal. The sequencing read is labeled as having the genetic variant if the reference match score and the variant match score indicate that the sequencing read more closely matches the corresponding variant sequence than the corresponding reference sequence. The sequencing read is labeled as not having the genetic variant if the reference match score and the variant match score indicate that the sequencing read more closely matches the corresponding reference sequence than the corresponding variant sequence. Finally, the sequencing read is labeled as an inconclusive read, e.g., inconclusive if the reference match score and the variant match score are equal. The labeled sequencing reads may be stored in the memory, or a number of sequencing reads having the genetic variant and/or a number of sequencing reads not having the genetic variant (and, optionally, the number of inconclusive reads) may be stored in the memory. In some embodiments, the computer-implemented process can use the number of sequencing reads labeled as having the genetic variant and/or the number of sequencing reads labeled as not having the genetic variant to call the sample as having the variant and/or determine a variant allele frequency for the sample. This process may be repeated for any number of genetic variants to be queried.

In some embodiments, a computer-implemented method of detecting a genetic variant or determining an allele frequency for the genetic variant in a test sample from a subject, comprising, and an electronic device comprising one or more processors and a memory storing a reference sequence that does not comprise the genetic variant and a variant sequence comprising the genetic variant at a variant locus; receiving, at the one or more processors, one or more sequencing reads associated with the test sample that corresponds with the reference sequence and the variant sequence; receiving, at the one or more processors, the reference sequence from the memory; generating, at the one or more processors, a reference match score for each of the one or more sequencing reads by aligning each sequencing read to the corresponding reference sequence; receiving, at the one or more processors, the variant sequence from the memory; generating, at the one or more processors, a variant match score for each of the one or more sequencing reads by aligning each sequencing read to the corresponding variant sequence; and labeling, at the one or more processors, each of the one or more sequencing reads as either having the genetic variant, not having the genetic variant, or a being an inconclusive read, based on the reference match score and the variant match score; wherein: a sequencing read is labeled as having the genetic variant if the reference match score and the variant match score indicate that the sequencing read more closely matches the corresponding variant sequence than the corresponding reference sequence; a sequencing read is labeled as not having the genetic variant if the reference match score and the variant match score indicate that the sequencing read more closely matches the corresponding reference sequence than the corresponding variant sequence; and a sequencing read is labeled as an inconclusive read if the reference match score and the variant match score are equal. In some embodiments, the method further comprises storing a label associated with each sequencing read in the memory.

In some embodiments, the computer-implemented method may further include calling, using the one or more processors, the presence of the genetic variant in the test sample based on the labeled one or more sequencing reads. The call for the genetic variant can be stored, by the one or more processors, in the memory.

In some embodiments, the computer-implemented method may further include, using the one or more processors, determining a variant allele frequency of the genetic variant in the test sample based on the labeled one or more sequencing reads. The variant allele frequency call may be stored in the memory.

The computer-implemented method may rely on the use of a variant panel stored in the memory to generate the reference sequence and/or the variant sequence used according to the method. The method may include selecting, using the one or more processors, the genetic variant from the variant panel, generating, using the one or more processors, the reference sequence and/or the variant sequence; and storing the reference sequence and/or the variant sequence in the memory. In other embodiments, the reference sequence and or the variant sequenced used according to the method is pre-stored in the memory, and corresponds to the queried genetic variant.

In some embodiments, the computer-implemented method includes the automatic generation or updating of a report (such as an electronic medical record). The report can include one or more of a call for the presence or absence of the genetic variant, a call for the variant allele frequency, and/or a disease status. The report can also include identifying information for the subject (e.g., name, identification number, etc.). The report may be stored in the memory and/or transmitted to a second electronic device (for example, an electronic device of the subject or a healthcare provider of the subject).

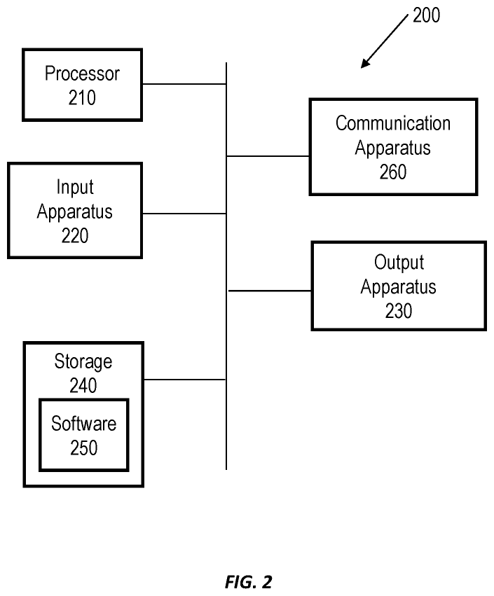

The techniques described herein can be implemented on one or more apparatuses. In some embodiments, an apparatus comprises one or more electronic devices. FIG. 2 shows an example of a computing device in accordance with one embodiment. Device 200 can be a host computer connected to a network. Device 200 can be a client computer or a server. As shown in FIG. 2, device 200 can be any suitable type of microprocessor-based device, such as a personal computer, workstation, server or handheld computing apparatus (portable electronic device) such as a phone or tablet. The device can include, for example, one or more of processor 210, input device 220, output device 230, storage 240, and communication device 260. Input device 220 and output device 230 can generally correspond to those described above, and can either be connectable or integrated with the computer.

Input device 220 can be any suitable device that provides input, such as a touch screen, keyboard or keypad, mouse, or voice-recognition device. Output device 230 can be any suitable device that provides output, such as a touch screen, haptics device, or speaker.

Storage 240 can be any suitable device that provides storage, such as an electrical, magnetic or optical memory including a RAM, cache, hard drive, or removable storage disk. Communication device 260 can include any suitable device capable of transmitting and receiving signals over a network, such as a network interface chip or device. The components of the computer can be connected in any suitable manner, such as via a physical bus or wirelessly.

Software 250, which can be stored in storage 240 and executed by processor 210, can include, for example, the programming that embodies the functionality of the present disclosure (e.g., as embodied in the devices as described above).

Software 250 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 240, that can contain or store programming for use by or in connection with an instruction execution system, apparatus, or device.

Software 250 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

Device 200 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 200 can implement any operating system suitable for operating on the network. Software 250 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example.

In an exemplary embodiment, there is an electronic device comprising: one or more processors; a memory; and one or more programs, wherein the one or more programs are stored in the memory and configured to be executed by the one or more processors, the one or more programs including instructions for: (a) selecting a genetic variant at a variant locus from a variant panel; (b) obtaining one or more sequencing reads associated with a test sample that overlaps the variant locus; (c) generating a reference match score for each of the one or more sequencing reads by aligning each sequencing read to a corresponding reference sequence, wherein the corresponding reference sequence does not comprise the genetic variant; (d) generating a variant match score for each of the one or more sequencing reads by aligning each sequencing read to a corresponding variant sequence, wherein the corresponding variant sequence comprises the genetic variant; and (e) labeling each of the one or more sequencing reads as either having the genetic variant, not having the genetic variant, or a being an inconclusive read, based on the reference match score and the variant match score; wherein: a sequencing read is labeled as having the genetic variant if the reference match score and the variant match score indicate that the sequencing read more closely matches the corresponding variant sequence than the corresponding reference sequence; a sequencing read is labeled as not having the genetic variant if the reference match score and the variant match score indicate that the sequencing read more closely matches the corresponding reference sequence than the corresponding variant sequence; and a sequencing read is labeled as an inconclusive read if the reference match score and the variant match score are equal.

In another exemplary embodiment, there is a non-transitory computer-readable storage medium storing one or more programs, the one or more programs comprising instructions, which when executed by one or more processors of an electronic device having a display, cause the electronic device to: (a) select a genetic variant at a variant locus from a variant panel; (b) obtain one or more sequencing reads associated with the test sample that overlaps the variant locus; (c) generate a reference match score for each of the one or more sequencing reads by aligning each sequencing read to a corresponding reference sequence, wherein the corresponding reference sequence does not comprise the genetic variant; (d) generate a variant match score for each of the one or more sequencing reads by aligning each sequencing read to a corresponding variant sequence, wherein the corresponding variant sequence comprises the genetic variant; and (e) label each of the one or more sequencing reads as either having the genetic variant, not having the genetic variant, or a being an inconclusive read, based on the reference match score and the variant match score; wherein: a sequencing read is labeled as having the genetic variant if the reference match score and the variant match score indicate that the sequencing read more closely matches the corresponding variant sequence than the corresponding reference sequence; a sequencing read is labeled as not having the genetic variant if the reference match score and the variant match score indicate that the sequencing read more closely matches the corresponding reference sequence than the corresponding variant sequence; and a sequencing read is labeled as an inconclusive read if the reference match score and the variant match score are equal.

### Model for Reducing Noise and Improving Detection Accuracy

Methods disclosed herein can provide a process for detecting a genetic variant and/or assessing a variant allele frequency of one or more samples obtained from a subject. A model, e.g., a probability model or distribution model, can be utilized to account for noise and improve accuracy of the methods. In some embodiments, noise may be introduced from sequencing a sample obtained from a subject to produce one or more sequencing reads and aligning the sequencing reads with a reference sequence. As a result of potential errors associated with sequencing reads, e.g., errors introduced by the sequencing and alignment processes, the some methods may incorrectly assign sequencing reads as alternate (e.g., variant) when the variant is not present in the sample data. That is, errors introduced via the sequencing and alignment processes can result in false positives—where the sequencing read is identified as variant, when in fact, the variant is not present in the sequencing read.

As used herein, noise can refer to one or more errors introduced into a sequencing read. In some embodiments, the errors can include one or more of sample preparation errors, amplification bias errors, and sequencing errors. For example, the sequencing process can introduce one or more errors into the sequencing read. For example, while sequencing the sample, the system may unintentionally introduce one or more of an insertion, deletion, substitution, or rearrangement into the sequencing read. In some instances, the alignment process can introduce one or more errors into the sequencing read. For example, the sequencing read may be misaligned with a corresponding reference sequence such that comparing the sequencing read with the references sequence produces the appearance of one or more of an insertion, deletion, substitution, or rearrangement in the sequencing read.

In some examples, the noise associated with a sequencing read can be locus specific. For example, in some embodiments, the alignment process can be sensitive to the sequence context of a variant at a variant locus. Accordingly, in some embodiments, accounting for noise associated with a sample can be locus specific. For example, in some embodiments, the model can be associated with one or more functions related to one of more sources of noise in a plurality of sequencing reads that overlap the variant locus. As noted above, the one or more sources of noise can include sample preparation errors, amplification bias errors, sequencing errors, alignment errors, or any combination thereof.

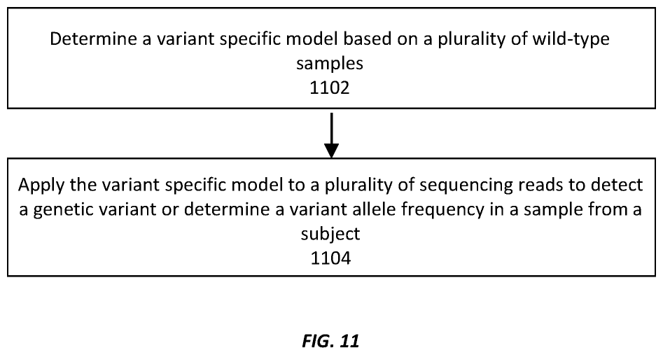

FIG. 11 shows an exemplary method for detecting a genetic variant or determine a variant allele frequency in a sample from a subject. At step 1102, a variant specific model can be determined based on one or more wild-type samples. The model can indicate the likelihood that the identified genetic variant is a true positive, as opposed to a false positive where sequencing reads from the wild-type sample (i.e., sequencing reads that do not include the variant) are detected as having the variant. In some embodiments, the variant specific model can be associated with one or more of a sequencing count, depth, or ratio of the two. As used herein, “sequencing count” can refer to a number of reads classified as supporting the presence of a prior baseline alteration. As used herein, the term “sequencing depth” can refer to a number of reads found at the locus of a prior baseline alteration. As used herein, a ratio of the sequencing count to the sequencing depth can be associated with a variant allele frequency (VAF). In one or more examples, reads that are equivocal (e.g., neither supporting the alteration or the reference genome) are excluded.

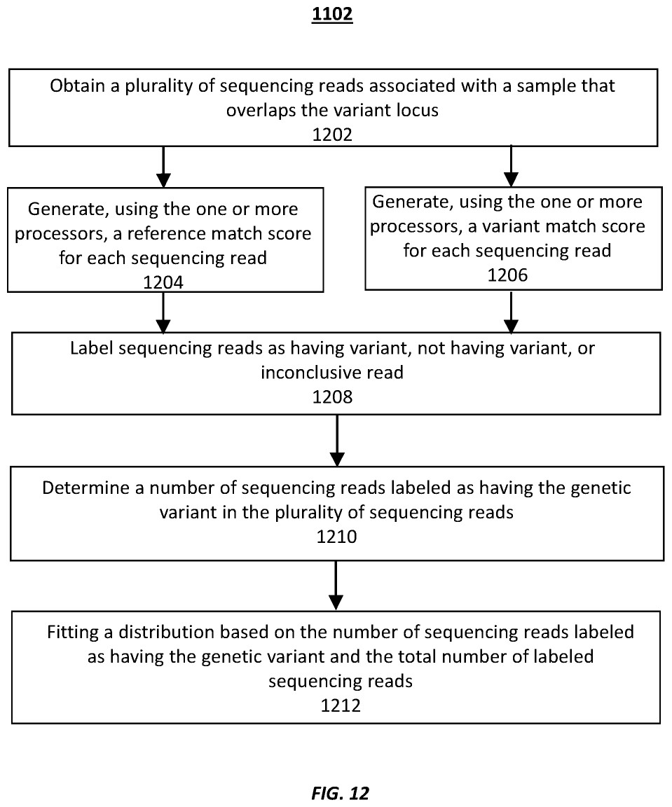

In some embodiments, the variant specific model can be determined with respect to a reference variant, e.g., a genetic variant selected from a variant panel as described above. For example, the wild-type samples can be selected to include the locus of the reference variant, but not include the variant itself, such that a wild-type sequencing read does not include the reference variant. In some embodiments, the sequencing reads that do not include the variant can be locus specific for each of the wild-type samples, e.g., the sequencing reads for each wild-type can correspond to the locus of the reference variant. In some embodiments, the one or more wild-type samples can correspond to a pool of wild-type samples. In some embodiments the wild-type pool can include 10-10,000 samples, for example, in some embodiments, the wild-type pool can include approximately 10 samples, approximately 100 samples, approximately 1,000 samples, approximately 10,000 samples, or approximately 100,000. A skilled artisan will understand that more or less samples can be included in the wild-type pool and that the size of the wild-type pool is not intended to limit the scope of the disclosure. Details of generating the model is described herein with reference to FIG. 12.

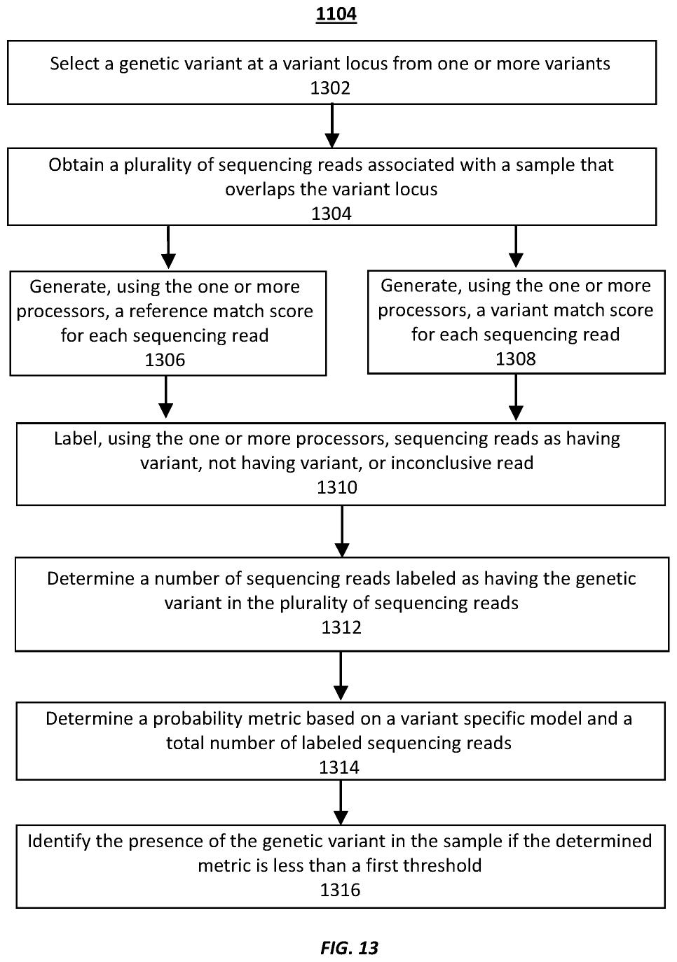

At step 1104, the variant specific model can be applied to a plurality of sequencing reads obtained from a sample from a subject. The variant specific model can be applied to the sequencing read generated from the sample to determine whether the sample includes the reference variant. In some embodiments, the variant specific model can be a locus specific model. For example, the variant specific model can be determined with respect to a pre-determined locus. Accordingly, the variant specific model can be applied to the variant locus of the sample, e.g., a corresponding locus on the sample. In some embodiments, the variant specific model may not be locus specific and can be applied to one or more variant loci. Details of applying the model is described herein with reference to FIGS. 13-15.

FIG. 12 shows an exemplary method for determining a variant specific model based on one or more wild-type samples (e.g., step 1102 of FIG. 11). At step 1202, sequencing reads that overlap the variant locus and are associated with the test sample are obtained. For example, sequencing reads can be generated by sequencing nucleic acid molecules in the sample. In some embodiments, these sequencing reads can be from a wild-type sample selected from the wild-type pool.

At step 1204, a reference match score for each sequencing read can be obtained by aligning the sequencing read to a corresponding reference sequence. At step 1206, a variant match score for each sequencing read can be generated by aligning the sequencing reads to a corresponding variant sequence. Using the reference match score and the variant match score, the sequencing reads can be labeled as at least one of having the variant, not having the variant, or inconclusive read at step 208. For example, a sequencing read may be labeled as having the variant if the reference match score and the variant match score indicate that the sequencing read more closely matches the variant sequence than the reference sequence. As another example, a sequencing read may be labeled as not having the variant if the reference match score and the variant match score indicate that the sequencing read more closely matches the reference sequence than the variant sequence. In some embodiments, a sequencing read may be labeled as inconclusive when the reference match score and a variant match score are equal. In some embodiments, a sequencing read may be labeled as inconclusive when the likelihood that a read should be labeled as a reference sequence and the likelihood that a read should be labeled as a variant are equal.

At step 1210, the number of sequencing reads labeled as having the variant can be determined for the plurality of sequencing reads. In some embodiments, the number of sequencing reads that are labeled as having the reference variant can be expressed as n; the total number of sequencing reads that are labeled as not having the reference variant can be expressed as z, and the inconclusive reads can be expressed as IC. As discussed above, the wild-type samples are selected because these samples do not include the reference variant. Based on this, one may expect the number of sequencing reads labeled as having the reference variant for a wild-type sample to be zero. However, in practice the number of sequencing reads labeled as having the genetic variant may be non-zero due to noise in the sequencing data. Accordingly, any non-zero value for the number of sequencing reads labeled as having the genetic variant from a wild-type sample may be attributed to noise.

At step 1212, a model, e.g., distribution model, can be fit based on the number of sequencing reads labeled as having the genetic variant in step 1210 and the total number of labeled sequencing reads. For example, a probability p that a sequencing read has been labeled as a variant from the wild-type sample (i.e., a false positive) can be determined. In some embodiments, the probability p that a sequencing read has been labeled as a variant can be expressed as \hat{p}=n/N, where N corresponds to the total number of labeled sequencing reads (e.g., N=n+ [symbol] +IC).

In some embodiments, the distribution can be fit (e.g., step 1212) based on the number of sequencing reads labeled as having the genetic variant and the total number of sequencing reads minus the number of sequencing reads labeled as inconclusive. According to such embodiments, the probability p that a sequencing read has been labeled as a variant can be expressed as \hat{p}=n/(N−IC), such that the number of inconclusive reads are excluded from the analysis. According to this latter embodiment, excluding the inconclusive reads from the probability metric can improve the accuracy because the inconclusive reads may not be indicative of whether the sample includes the variant.

In some embodiments, the distribution can be fit based on the probability of two or more samples, e.g., two or more samples from the wild-types pool. For example, steps 1202 to 1210 can be repeated with respect to a second sample from the wild-types pool to obtain determine a second probability that a sequencing read has been labeled as a variant. The distribution can then be fit to the set of probabilities determined from the samples from the wild-types pool. The number of samples used to fit the distribution is not intended to limit this disclosure, and a skilled artisan will understand that any number of samples selected from the wild-type pool can be used to determine a corresponding probability and fit the distribution. For example, if the number of sequencing reads labeled as variant n, is treated as an outcome of a Bernoulli process, the probability of finding n sequencing reads from N sequencing reads can be expressed as B (n; \hat{p}, N), where B is the binomial distribution. In some embodiments, the probability of finding n sequencing reads from N−IC sequencing reads can be expressed as B (n; \hat{p}, N−IC), where B is the binomial distribution.

In some embodiments, the distribution can be fit based on the probability of two or more samples, e.g., two or more samples from the wild-types pool. For example, steps 1202 to 1210 can be applied to a sample pool that includes two or more samples selected from the wild-types pool to obtain determine a probability that sequencing reads from the two or more samples have been labeled as a variant. The distribution can then be fit based on the probability determined from the pooled samples. The number of samples included in the pool is not intended to limit this disclosure, and a skilled artisan will understand that any number of samples selected from the wild-type pool can be used to determine a corresponding probability and fit the distribution. For example, if the number of sequencing reads from the sample pool labeled as variant n, is treated as an outcome of a Bernoulli process, the probability of finding n sequencing reads from N sequencing reads can be expressed as B (n; \hat{p}, N), where B is the binomial distribution. In some embodiments, the probability of finding n sequencing reads from N−IC sequencing reads can be expressed as B (n; \hat{p}, N−IC), where B is the binomial distribution.

In some examples, an exemplary distribution can be fit based on the method described with respect to FIG. 12. For example, a resulting model fit based on the exemplary distribution can correspond to the distribution fit based on the calculated metric for one or more samples from the wild-type pool. The model y-axis can correspond to the probability q that the observed number of sequencing reads labeled as variant (expressed as m) from the total number of sequencing reads (expressed as M) is derived from noise. For example, the model can be configured to receive m/M to determine q. In some embodiments, the model is configured to receive m/(M−IC) to determine q.

In some examples, the probability distribution e.g., variant specific model, can be used to determine one or more thresholds. The one or more thresholds can be used when evaluating a sample from a subject to account for noise. For example, the thresholds can be used to detect a genetic variant or determine a variant allele frequency in a sample from a subject. In some examples, a single threshold can be used to identify a sequencing read as having the variant or not having the variant. In some examples, at least two thresholds can be used to identify a sequencing read as having the variant, not having the variant, or inconclusive. In some embodiments, the thresholds can be variant specific, that is, the thresholds can be separately determined for each variant. For example, the thresholds between variants may differ. In some embodiments, the thresholds can be consistent between variants. Details of using the thresholds is described herein with reference to FIG. 13.

In some embodiments, different probability distributions can be determined for different variant loci. For example, in some embodiments, step 1102 can be performed with respect to a first variant locus and repeated with respect to a second variant locus. In this manner, to the extent that the noise differs between the first variant locus and the second variant locus, the variant specific model can account for this difference.

Although the example above is discussed with respect to the Binomial distribution, a skilled artisan will understand that other functions can be used without departing from the scope of this disclosure. For example, the variant specific model can be associated with one or more functions that have been fitted to data for a plurality of sequencing reads that overlap the variant locus. For example, one or more of uniform distribution functions, Poisson distribution functions, negative binomial distribution functions, normal distribution functions, log-normal distribution functions, Cauchy-Lorentz distribution functions, log-logistic distribution functions, exponential distribution functions, gamma distribution functions, hypergeometric distribution functions, etc. can be used without departing from the scope of this disclosure. In some embodiments, the probability distribution can be associated with one or more functions related to one of more sources of noise in a plurality of sequencing reads that overlap the variant locus. In some embodiments, the probability distribution can be associated with one or more functions that have been fitted to data for a plurality of sequencing reads that overlap the variant locus.

In some embodiments, a mechanistic approach to determine the probability distribution, e.g., variant specific model, can be used. For example, based on the mechanistic approach, the specific sources of noise (e.g., sequencing errors, amplification (PCR) errors, and alignment errors) at each locus can be analyzed. For instance, the specific molecular errors due to the chemistry used for amplification and sequencing, sequencing artifacts, and/or sequencing errors can examined and modeled for a specific locus, e.g., according to step 1102. In one or more examples, these separate models can then be combined in a single composite model or distribution. In some embodiments, the one or more models related to specific sub-processes can be used to reduce the impact of various errors (e.g., sequencing errors and PCR errors) by implementing one or more error correction schemes such as unique molecular identifier (UMIs) and fitted background correction (FBCs).

In some embodiments, an empirical approach can be used. For example, based on the empirical approach, a large number of sequencing reads can be collected and examined, e.g., according to step 1102, and the resulting data can be fit to one or more functions, e.g., uniform distribution functions, binomial distribution functions, Poisson distribution functions, negative binomial distribution functions, normal distribution functions, log-normal distribution functions, Cauchy-Lorentz distribution functions, log-logistic distribution functions, exponential distribution functions, gamma distribution functions, hypergeometric distribution functions, or any combination thereof. For instance, the variant specific model may be represented by a sum of three different binomial distributions.

In some embodiments, one or more thresholds can be determined empirically based on the probability model. In some embodiments, one or more thresholds, e.g., a first and/or second threshold, can be determined empirically using the probability model, such that the one or more thresholds can be set to a value that corresponds to a specified confidence level that a sequencing read labeled as not having the genetic variant is correct. For example, in some embodiments, the confidence level can be about 90% or 95%, although confidence levels greater than, less than, or ranges, can be used without departing from the scope of this disclosure. In some embodiments, one or more thresholds can be determined empirically based on clinical trial outcomes. In some embodiments, one or more thresholds can be determined using a Kaplan-Meier estimator and data associated with samples from a plurality of subjects. For example, the Kaplan-Meier estimator can be used to maximize the difference between outcome data for a set of patients that have the variant and a second set of patients that do not have the variant by providing a variable, e.g., sliding, threshold value. For example, the one or more threshold values could be adjusted and, as a result, the classification of a sample may change, e.g., move from not having the variant to inconclusive and/or to having the variant. In some embodiments, the Kaplan-Meier outcomes can be used to classify a subject based on the determination of whether the subject's sample is detected as having a genetic variant with respect to one or more variants. For example, the Kaplan-Meier process could separate subjects into “responders” and “non-responders” (e.g., responsive to treatment or non-responsive to treatment) based on >=X variants (e.g., where X=2) determined to be variant in >=Y samples (where Y=1 or Y=2). In some embodiments, one or more thresholds can be determined using the Cox proportional hazards model. For example, the Cox proportional hazards model is a parametric model that can assume that the hazards of the treated vs untreated are proportional to one another. With mathematical formulation, the hazard ratio can be estimated by using the covariates in the model. In some embodiments, the user to specify the model and estimate the hazards ratio using software.

FIG. 13 shows an exemplary method for applying a variant specific model to a plurality of sequencing reads, to detect a genetic variant or determine a variant allele from a sample from a subject (e.g., step 1104 from FIG. 11). At step 1302, a genetic variant at a variant locus can be selected from one or more variants. In some embodiments, the one or more variants can be selected from a variant panel. The variant panel can be a personalized variant panel. As discussed above, a personal variant panel can be established for a subject using an initial sample, e.g., baseline sample. The personalized variant panel can include genetic variants that may be indicative of a disease. In some embodiments, the genetic variant can be selected based on one or more variants identified in the baseline sample. In some embodiments, the one or more variants can be selected from variants identified in literature. In some embodiments, the one or more variants can be selected from variants identified empirically, e.g., identified in a clinical trial.

At step 1304, sequencing reads associated with a sample that overlaps the variant locus can be obtained. Sequencing reads can be generated by sequencing nucleic acid molecules in the sample. For example, a time point sample can include M sequencing reads. The sample can be obtained from a subject, e.g., the subject that provided the baseline sample. A reference match score for each sequencing read can be obtained by aligning the sequencing reads to a reference sequence at step 1306, and a variant match score for each sequencing read can be generated by aligning the sequencing reads to a corresponding variant sequence at step 1308.

Using the reference match score and the variant match score, the sequencing reads can be labeled as at least one of having the variant, not having the variant, or inconclusive read at step 1310. In some embodiments, M can correspond to a total number of labeled sequencing reads. For example, a sequencing read may be labeled as having the variant if the reference match score and the variant match score indicate that the sequencing read more closely matches the variant sequence than the reference sequence. As another example, a sequencing read may be labeled as not having the variant if the reference match score and the variant match score indicate that the sequencing read more closely matches the reference sequence than the variant sequence. In some embodiments, a sequencing read may be labeled as inconclusive when the reference match score and a variant match score are equal.

At step 1312, the number of sequencing reads labeled as having the variant in the plurality of sequencing reads can be determined. In some embodiments, the number of sequencing reads labeled as having the variant can correspond to m. Accordingly, the number of sequencing reads labeled as not having the variant can correspond to M−m.

At step 1314, a probability metric can be determined based on the number of sequencing reads labeled as having the genetic variant (m) and a total number of labeled sequencing reads (M). In some embodiments, the probability metric is a statistical value indicative of a likelihood that the genetic variant is detected due to the presence of the genetic variant in the sample rather than noise. In some embodiments, the probability metric can be indicative of whether the number of sequencing reads labeled as variants differs from the number of sequencing reads labeled as variants due to noise. In this manner, the statistical value, e.g., probability metric can be used to improve the accuracy of the results of a sequencing read by discounting sequencing reads labeled as variant due to noise.

In some embodiments, the probability metric can be a p-value. For example, in some embodiments, the probability metric can correspond to the output of a variant specific model. For example, the probability metric can be obtained based on a binomial distribution by determining q=B (m; \hat{p}, M), where \hat{p}=m/M. In such embodiments, the distribution may be associated with a metric determined based on n/N. In some embodiments, the probability metric can exclude sequencing reads labeled as inconclusive. In such embodiments, the probability metric can be obtained based on a binomial distribution by determining q=B (m; \hat{p}, (M−null)), where \hat{p}=m/(M−IC), as discussed with respect to step 1212. In such embodiments, the distribution, e.g., variant specific model, may be associated with a metric determined based on n/(N−IC), as discussed with respect to step 1212.

A skilled artisan will understand that other distributions and/or functions can be used to determine the probability metric without departing from the scope of this disclosure, e.g., such as uniform distribution functions, Poisson distribution functions, negative binomial distribution functions, normal distribution functions, log-normal distribution functions, Cauchy-Lorentz distribution functions, log-logistic distribution functions, exponential distribution functions, gamma distribution functions, hypergeometric distribution functions, etc., or any combination thereof. In some embodiments, the probability metric can be locus specific. In some embodiments, the probability metric may not be locus specific.

At step 1316, the presence of the genetic variant in the sample can be determined if the probability metric is less than a first threshold (TO). As discussed above, in some embodiments, the probability can correspond to an output of the variant specific model. In some embodiments, the probability metric can be compared to a second threshold (T1). In some embodiments, if the determined probability metric is greater than or equal to the second threshold, the sample may be identified as lacking the genetic variant, e.g., the genetic variant is absent from the sample. If the determined probability metric is greater than or equal to the first threshold and less than the second threshold, then the sample may be identified as inconclusive. In some embodiments, the first threshold can be approximately 0.05 (e.g., T0=0.05) and the second threshold can be approximately 0.1 (e.g., T0=0.1). A skilled artisan will understand that other values for the one or more thresholds can be used without departing from the scope of the present disclosure.

In some embodiments, the first threshold and/or the second threshold can be variant specific. In some embodiments, the first threshold and/or the second threshold can be locus specific. For example, the threshold can be determined with respect to a specific genetic variant at a specific locus. As discussed above, in some embodiments, one or more thresholds can be determined from the probability model determined in step 1102, described in FIG. 12.

In some embodiments, a second genetic variant can be detected in the sample from the subject. For example, the step 1104 described in FIG. 13 can further include, labeling sequencing reads associated with the sample for a second genetic variant selected from the variant panel. Next, a second probability metric can be determined using a variant specific model for the second variant and a total number of labeled sequencing reads for the second genetic variant. The number of labeled sequencing reads identified as the second genetic variant can be expressed as m₂, while the number of labeled sequencing reads identified as the first genetic variant can be expressed as m₁. For example, in some embodiments, the second probability metric can correspond to the output of the variant specific model. For example, the probability metric can be obtained based on a distribution by determining q=B (m₂; \hat{p}, M), where \hat{p}=m₂/M. In such embodiments, the distribution may be associated with a metric determined based on n/N. In some embodiments, the probability metric can be obtained based on a binomial distribution by determining q=B (m₂; \hat{p}, (M−null)), where \hat{p}=m₂/(M−IC), as discussed with respect to step 1212. In such embodiments, the distribution, e.g., variant specific model, may be associated with a metric determined based on n/(N−IC), as discussed with respect to step 1212.

The determined second probability metric for the second genetic variant can be compared to a third threshold (T2). If the determined probability metric for the second genetic variant is less than the third threshold, the sample can be identified as including the second genetic variant. In some embodiments, labeling the sequencing reads associated with the sample for the second genetic variant can be locus specific. For example, the labeling the sequencing reads associated with the sample for the second genetic variant can be associated with a different locus than the initial genetic variant.

In some embodiments, the probability metric can be compared to a fourth threshold (T3). In some embodiments, if the determined probability metric is greater than or equal to the fourth threshold, the sample may be identified as lacking the genetic variant, e.g., the genetic variant is absent from the sample. If the determined probability metric is greater than or equal to the third threshold and less than the fourth threshold, then the sample may be identified as inconclusive or inconclusive. In some embodiments, the third threshold can be, for example, approximately 0.05 (e.g., T2=0.05) and the fourth threshold could be, for example, approximately 0.1 (e.g., T3=0.1). In some embodiments, the third and fourth thresholds may be equal to the first and second thresholds, respectively. In some embodiments, the third and fourth thresholds may differ from the first and second thresholds, respectively. A skilled artisan will understand that the one or more thresholds, e.g., the first through fourth thresholds, can correspond to various values without departing from the scope of the present disclosure.

In some embodiments, using a baseline sample from the subject to determine the one or more variants and/or variant panel (e.g., in step 1302) can improve sensitivity of detecting a genetic variant or determining a variant allele frequency in a sample from a subject. For example, baseline informed approaches are inherently more sensitive than non-baseline informed approaches because it benefits from awareness of specific biomarker characteristics of the subject and avoids the multiple testing challenges associated with making non-baseline-informed assessments. In this manner, using the locus specific noise model can optimize noise assessments and system performance for the local variant in the genome of a subject. For example, the disclosed method can provide a statistically meaningful way to improve variant allele frequency estimates by accounting for noise and/or locus specific noise in the sequencing reads.

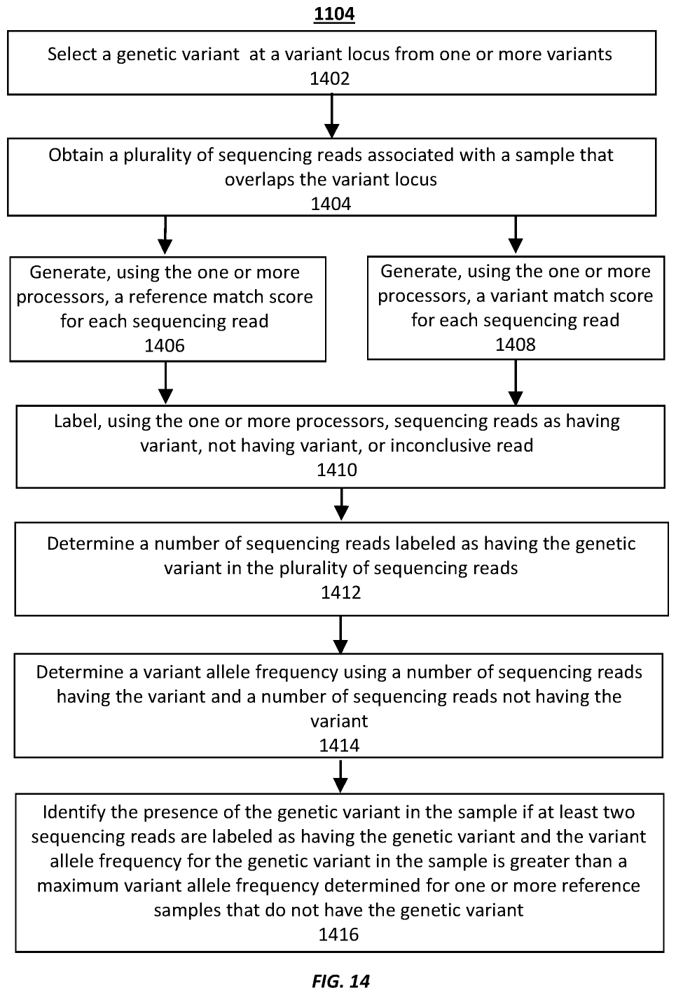

FIG. 14 shows an exemplary method for applying a variant specific model to a plurality of sequencing reads, where the sequencing reads are obtained from a sample from a subject (e.g., step 1104 from FIG. 11). Steps 1402-1412 may be substantially similar to steps 1302-1312. At step 1414, the variant allele frequency can be determined using the number of sequencing reads having the variant and the number of sequencing reads not having the variant. At step 1416, the presence of the genetic variant in the sample can be identified as having the genetic variant (e.g., positive) if at least two sequencing reads are labeled as having the genetic variant and the variant allele frequency for the genetic variant in the test sample is greater than a maximum variant allele frequency determined for one or more references samples that do not have the genetic variant. In some embodiments, the test sample is identified as not having the genetic variant (e.g., negative) if the variant allele frequency for the genetic variant in the test sample is less than a specified confidence level for determinations of variant allele frequency in one or more reference samples that do not have the genetic variant. In some embodiments, the confidence level can correspond to 95%. The sample can be determined to be inconclusive if the sample is identified as neither positive or negative.

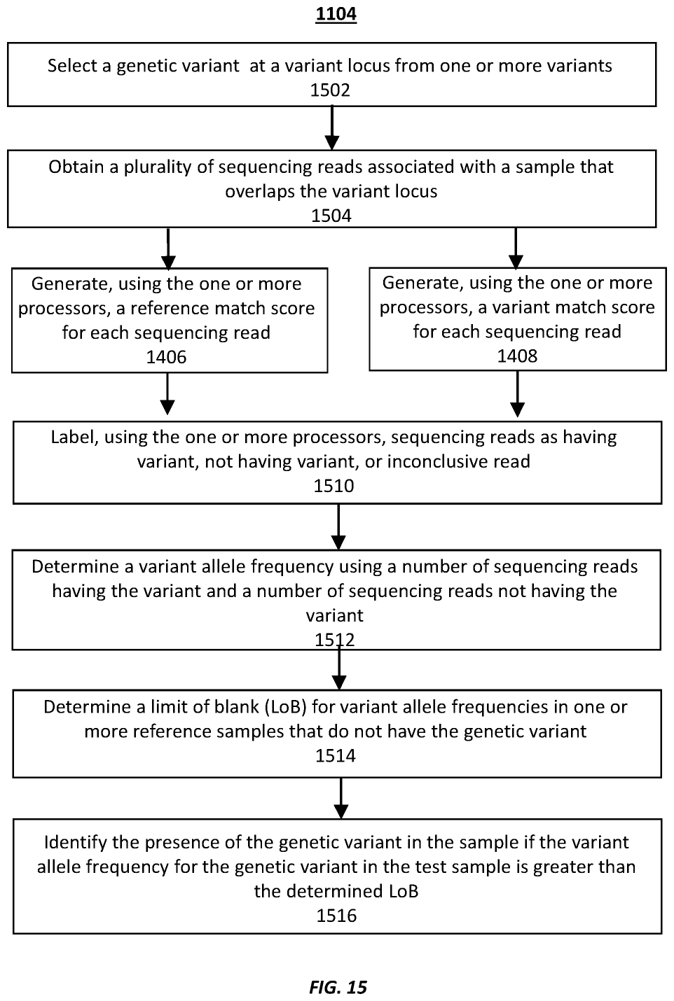

FIG. 15 shows an exemplary method for applying a variant specific model to a plurality of sequencing reads, where the sequencing reads are obtained from a sample from a subject (e.g., step 1104 from FIG. 11). Steps 1502-1510 may be substantially similar to steps 1302-1310. At step 1512, the variant allele frequency can be determined using the number of sequencing reads having the variant and the number of sequencing reads not having the variant. At step 1514, a limit of blank (LoB) for variant allele frequencies in one or more reference samples that do not have the genetic variant can be determined. At step 1516, the test sample can be identified as having the genetic variant if the variant allele frequency for the genetic variant in the test sample is greater than the LoB. In some embodiments, the test sample can be identified as not having the genetic variant or inconclusive if the variant allele frequency for the genetic variant in the test sample is less than or equal to the LoB.

In some embodiments, variants in the variant panel can be associated with a reference sequence and a corresponding variant sequence that can include the locus of the variant with left and right flanking regions (e.g., a 5′ flanking region and a 3′ flanking region). The left and right flanking regions of the variant locus can provide context for the variant, and are the same for both the reference sequence and the corresponding variant sequence. Thus, the reference sequence and the corresponding variant sequence may be identical except for the variant itself. The corresponding variant sequence may include the variant, and the reference sequence may not include the variant (i.e., it includes the reference or “wild-type” sequence at the location of the variant). In some embodiments, the flanking regions can each include about 5 bases or more, about 10 bases or more, about 15 bases or more, about 20 bases or more, about 25 bases or more, about 30 bases or more, about 50 bases or more, about 75 bases or more, about 100 bases or more, about 150 bases or more, about 200 bases or more, about 250 bases or more, about 300 bases or more, about 400 bases or more, or about 500 bases or more. In some embodiments, the flanking regions can each include between about 5 bases and about 5000 bases, such as about 5 to about 10 bases, about 10 to about 20 bases, about 20 to about 50 bases, about 50 to about 100 bases, about 100 to about 200 bases, about 200 to about 500 bases, about 500 to about 1000 bases, about 1000 bases to about 2500 bases, or about 2500 bases to about 5000 bases. In some embodiments, the left and right flanking regions can have the same number of bases, and in some embodiments, the left and right flanking regions can have a different number of bases.

The reference sequence and the corresponding variant sequence can be generated, for example, using the reference sequence used to identify the variant (which may be a personalized reference sequence or a standard reference sequence). To generate the corresponding variant sequence, the variant can be selected and right and left flanking sequences can be added to the variant using the reference sequence. To generate the reference sequence, the reference sequence can be used using the same base locations as the corresponding variant sequence. Thus, in some embodiments, the reference sequence and corresponding variant sequence may be identical except for the genetic variant.

In some embodiments, the methods disclosed herein can include determining a disease status for a subject. In some embodiments, the disease can be cancer. In some embodiments, the disease status can include a qualitative factor indicating recurrence of a cancer in the subject, the presence of a cancer resistant to a treatment modality in the subject, or the presence of a cancer that can be treated with a particular treatment modality. In some embodiments, the disease status is quantitatively assessed (e.g., a determined tumor fraction of cfDNA, or a maximum somatic allele fraction of cfDNA). For example, the disease status may be a value proportional to a percentage of circulating-tumor DNA (ctDNA) compared to total cell-free DNA (cfDNA) in the test sample. For example, the disease status may be a maximum somatic allele fraction of cfDNA. Accordingly, in some embodiments, the sample can include cfDNA.

In some embodiments, the reference match score and the variant match score are determined using a sequence alignment algorithm. In some embodiments, the reference match score and the variant match score are determined using a Smith-Waterman alignment algorithm. In some embodiments, the reference match score and the variant match score are determined using a Needleman-Wunsch alignment algorithm.

In some embodiments, the variant panel can be determined by sequencing nucleic acid molecules in a previous sample obtained from the subject, and identifying one or more genetic variants. In some embodiments, the variant can be a somatic mutation. In some embodiments, the variant can be a germline mutation. In some embodiments, the genetic variant can include a single nucleotide variant (SNV), a multiple nucleotide variant (MNV), an indel, or a rearrangement junction.

In some embodiments, the subject may have received an intervening treatment for a disease between a previous sample being obtained and a current sample being obtained. In some embodiments, treatment can be adjusted based on a difference between a disease status for the subject determined using the sample and a previous disease status for the subject based on the previous sample. In some embodiments, the method can further include administering an anti-cancer agent or applying an anti-cancer treatment to the subject based on the generated genomic profile. An anti-cancer agent or anti-cancer treatment can refer to a compound that is effective in the treatment of cancer cells.

In some embodiments, the presence of a genetic variant in the sample can be determined, applied, and/or identified as a diagnostic value associated with the sample. In some embodiments, the presence of a genetic variant at one or more genomic loci of the sample can be used in generating a genomic profile for the subject (i.e., information about the subject's genome), which may then be analyzed to detect the presence of disease, to monitor the progression of disease, or to predict the risk of disease. In some embodiments, the presence of a genetic variant at one or more genomic loci of the sample can be used in making suggested treatment decisions for the subject. In some embodiments, the genomic profile may be comprehensive, e.g., comprising information about the presence of variant sequences at one or more genomic loci as identified through comprehensive genomic profiling (CGP), a next-generation sequencing (NGS) approach used to assess hundreds of genes (including relevant cancer biomarkers) in a single assay. In some embodiments, the genomic profile may be customized, e.g., comprising information about the presence of variant sequences at one or more selected genomic loci.

In some embodiments, a method of detecting a genetic variant or determining a variant allele frequency in a sample from a subject includes providing a plurality of nucleic acid molecules obtained from a sample from a subject, wherein the plurality of nucleic acid molecules comprises a mixture of tumor nucleic acid molecules and non-tumor nucleic acid molecules. Optionally, one or more adapters can be ligated onto one or more nucleic acid molecules from the plurality of nucleic acid molecules. In some embodiments, nucleic acid molecules from the plurality of nucleic acid molecules can be amplified. In some embodiments, nucleic acid molecules from the amplified nucleic acid molecules can be captured, wherein the captured nucleic acid molecules are captured from the amplified nucleic acid molecules by hybridization to one or more bait molecules. In some embodiments, the captured nucleic acid molecules can be sequenced, by a sequencer, to obtain a plurality of sequencing reads associated with the sample that overlap a variant locus of the genetic variant. In some embodiments, using one or more processors, a reference match score can be generated for each of the plurality of sequencing reads by aligning each sequencing read to a reference sequence that does not comprise the genetic variant. Using the one or more processors, a variant match score for each of the plurality of sequencing reads can be generated by aligning each sequencing read to a variant sequence that comprises the genetic variant. In some embodiments, using the one or more processors, each of the plurality of sequencing reads can be labeled as at least one of having the genetic variant, as not having the genetic variant, or as being an inconclusive read based on the reference match score and the variant match score of the respective sequencing read. In some embodiments, using the one or more processors, a number of sequencing reads labeled as having the genetic variant in the plurality of sequencing reads can be determined. In some embodiments, using the one or more processors, a probability metric based on a variant specific model and a total number of labeled sequencing reads can be determined. In some embodiments, using the one or more processors, the presence of the genetic variant in the sample can be identified if the determined probability metric is less than a first threshold.

In some embodiments, the variant specific model can be locus specific. In some embodiments, the first threshold is locus specific and variant specific. In some embodiments, detecting a genetic variant or determining a variant allele frequency in a sample from a subject can also include comparing, using the one or more processors, the determined probability metric to a second threshold, and either identifying the absence of the genetic variant in the sample if the determined probability metric is greater than or equal to the second threshold or identifying the presence or absence of the genetic variant in the sample as inconclusive if the determined probability metric is greater than or equal to the first threshold and less than the second threshold.

In some embodiments, the subject can be a cancer patient. In some embodiments, the sample can be obtained from the subject. In some embodiments, the sample can include a tissue biopsy sample, a liquid biopsy sample, a circulating tumor cell (CTC) sample, a cell-free DNA (cfDNA) sample, or a normal control. In some embodiments, the sample can be a liquid biopsy sample and comprise blood, plasma, cerebrospinal fluid, sputum, stool, urine, or saliva. In some embodiments, the tumor nucleic acid molecules can be derived from a tumor portion of a heterogeneous tissue biopsy sample, and the non-tumor nucleic acid molecules can be derived from a normal portion of the heterogeneous tissue biopsy sample. In some embodiments, the tumor nucleic acid molecules can be derived from a circulating tumor DNA (ctDNA) fraction of a cell-free DNA sample, and the non-tumor nucleic acid molecules can be derived from a non-tumor fraction of the cell-free DNA sample. In some embodiments, the one or more adapters can include comprise amplification primers or sequencing adapters. In some embodiments, the one or more bait molecules can include one or more nucleic acid molecules, each comprising a region that is complementary to a region of a captured nucleic acid molecule.

In some embodiments, amplifying nucleic acid molecules includes performing a polymerase chain reaction (PCR) amplification technique, non-PCR amplification technique, or isothermal amplification technique. In some embodiments, isothermal amplification techniques can include at least one selected from nicking endonuclease amplification reaction (NEAR), transcription mediated amplification (TMA), loop-mediated isothermal amplification (LAMP), helicase-dependent amplification (HDA), clustered regularly interspaced short palindromic repeats (CRISPR), strand displacement amplification (SDA). In some embodiments, the sequencing comprises use of a next generation sequencing (NGS) technique. In some embodiments, the sequencer can include a next generation sequencer.

In some embodiments, methods disclosed herein can include generating, by the one or more processors, a report indicating the tumor fraction of the sample. In some embodiments, methods disclosed herein can include transmitting the report to a healthcare provider. In some embodiments, the report can be transmitted via a computer network or a peer-to-peer connection.

In some embodiments, a method for detecting a disease state in a sample from a subject, can include sequencing nucleic acid molecules in the sample acquired from the subject to generate a plurality of sequencing reads and detecting a genetic variant of determining a variant allele frequency in the sample according to the methods described above, e.g., methods discussed with respect to FIGS. 11-15.

In some embodiments, a method of monitoring disease progression or recurrence can include sequencing nucleic acid molecules in a first sample acquired from a subject with a disease to generate a first set of sequencing reads and generating a personalized variant panel for the subject. The method can include sequencing nucleic acid molecules in a second sample acquired from the subject at a later time point than the first sample to generate a second set of sequencing reads. The method can include detecting, using the second set of sequencing reads, a genetic variant or determining, using the second set of sequencing reads, a variant allele frequency according to the methods described above, e.g., methods discussed with respect to FIGS. 11-15.

In some embodiments, the method of monitoring disease progression or recurrence can further include administering a disease therapy to the subject after the first sample is acquired from the subject and before the second sample is acquired from the subject. In some embodiments, the method of monitoring disease progression or recurrence can include determining a first disease status based on a number of sequencing reads in the first set of sequencing reads labeled as having a genetic variant from the variant panel and determining a second disease status based on a number of sequencing reads in the second set of sequencing reads labeled as having the genetic variant from the variant panel. In some embodiments, the method of monitoring disease progression or recurrence can further include determining disease progression by comparing the first disease status and the second disease status. In some embodiments, the method of monitoring disease progression or recurrence can further include administering a disease therapy to the subject after the first sample is acquired from the subject and before the second sample is acquired from the subject and adjusting the disease therapy based on the determined disease progression.

In some embodiments, a method of treating a subject with a disease can include acquiring a first sample from the subject, sequencing nucleic acid molecules in a first sample to generate a first set of sequencing reads, determining a first disease status using the first set of sequencing reads, generating a personalized variant panel for the subject, and administering a disease therapy to the subject. The method of treating a subject with a disease can further include acquiring a second sample from the subject after the disease therapy has been administered to the subject, sequencing nucleic acid molecules in the second sample to generate a second set of sequencing reads, detecting, using the second set of sequencing reads, the genetic variant or determining, using the second set of sequencing reads, the variant allele frequency according to the methods e.g., methods discussed with respect to FIGS. 11-15. The method of treating a subject with a disease can further include determining a second disease status based on the second set of sequencing reads, determining disease progression by comparing the first disease status and the second disease status, adjusting the disease therapy administered to subject based on the disease progression, and administering the adjusted disease therapy to the subject.

In some embodiments, the disease can be cancer. In some embodiments, the sample can be derived from a liquid biopsy sample from the subject. In some embodiments, the sample can be derived from a solid tissue sample, liquid tissue sample, or hematological sample, from the subject.

In some embodiments, methods disclosed herein can include sequencing nucleic acid molecules extracted from the sample to generate the plurality of sequencing reads. In some embodiments, methods disclosed herein can include generating or updating a report comprising (1) identifying information for the subject, and (2) a call for the presence or absence of the genetic variant, or a call for the variant allele frequency for the genetic variant. In such an embodiment, the method can further include transmitting the report to the subject or a healthcare provider for the subject.

Embodiments disclosed herein may include an electronic apparatus including at least one or more processors, a memory, and one or more programs. The one or more programs can be stored in the memory and configured to be executed by the one or more processors. The one or more programs can include instructions for selecting a genetic variant at a variant locus from a variant panel, obtaining a plurality of sequencing reads associated with a sample that overlap the variant locus, generating a reference match score for each of the plurality of sequencing reads by aligning each sequencing read to a reference sequence that does not comprise the genetic variant, generating a variant match score for each of the plurality of sequencing reads by aligning each sequencing read to a variant sequence that comprises the genetic variant, labeling each of the one or more sequencing reads as at least one of having the genetic variant, as not having the genetic variant, or as being an inconclusive read based on the reference match score and the variant match score of the respective sequencing read, determining a number of sequencing reads labeled as having the genetic variant, determining a probability metric based on a variant specific model and a total number of labeled sequencing reads, and identifying, using the one or more processors, the presence of the genetic variant in the sample if the determined probability metric is less than a first threshold.

Embodiments disclosed herein may include a non-transitory computer-readable storage medium storing one or more programs. The one or more programs can include instructions, which when executed by one or more processors of an electronic device, cause the electronic device to select a genetic variant at a variant locus from one or more variants, obtain a plurality of sequencing reads associated with a sample that overlaps the variant locus, generate a reference match score for each of the plurality of sequencing reads by aligning each sequencing read to a reference sequence that does not comprise the genetic variant, generate a variant match score for each of the plurality of sequencing reads by aligning each sequencing read to a variant sequence that comprises the genetic variant, label each of the plurality of sequencing reads as at least one of having the genetic variant, as not having the genetic variant, or as being an inconclusive read based on the reference match score and the variant match score of the respective sequencing read, determine a number of sequencing reads labeled as having the genetic variant, determine a probability metric based on a variant specific model and a total number of labeled sequencing read, and identify the presence of the genetic variant in the sample if the determined probability metric is less than a first threshold.

Embodiments disclosed herein may include a computer system including a processor and a memory communicatively coupled to the processor. The memory can be configured to store instructions that, when executed by the processor cause the processor to perform a method of detecting a genetic variant or determining a variant allele frequency in a sample from a subject according to any of the methods described above, e.g., with respect to FIGS. 11-15.

### EXAMPLES

The examples provided herein are included for illustrative purposes only and are not intended to limit the scope of the invention.

### Example 1

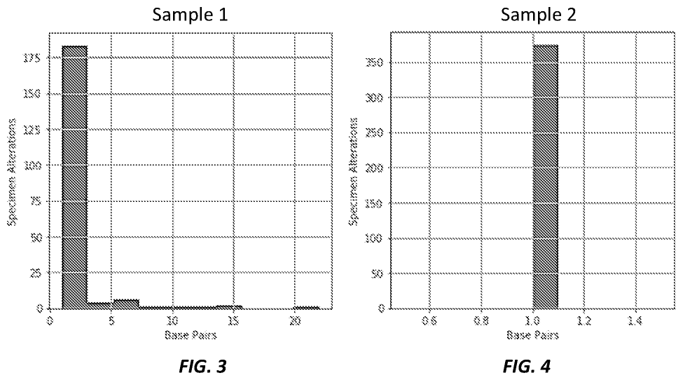

Sequencing reads from Sample 1 and Sample 2 were initially obtained using targeted sequencing methods and variants and allele depths called using standard variant calling protocols to generate curated sets of variants from the baseline sample. Variant panels and allele depths were selected for Sample 1 and Sample 2. Variants in the variant panel for Sample 1 ranged from 1 to 22 bases in length (FIG. 3), and variants in in the variant panel for Sample 2 included only variants of a single base length (FIG. 4).

Reference sequences corresponding to each variant in the variant panel (i.e., a reference sequence) and a variant sequence corresponding to each variant in the variant panel (i.e., a variant reference sequence) were generated. The variant or reference base(s) were flanked with 200 bases on each side of the variant locus to generate the corresponding variant sequence and the reference sequence.

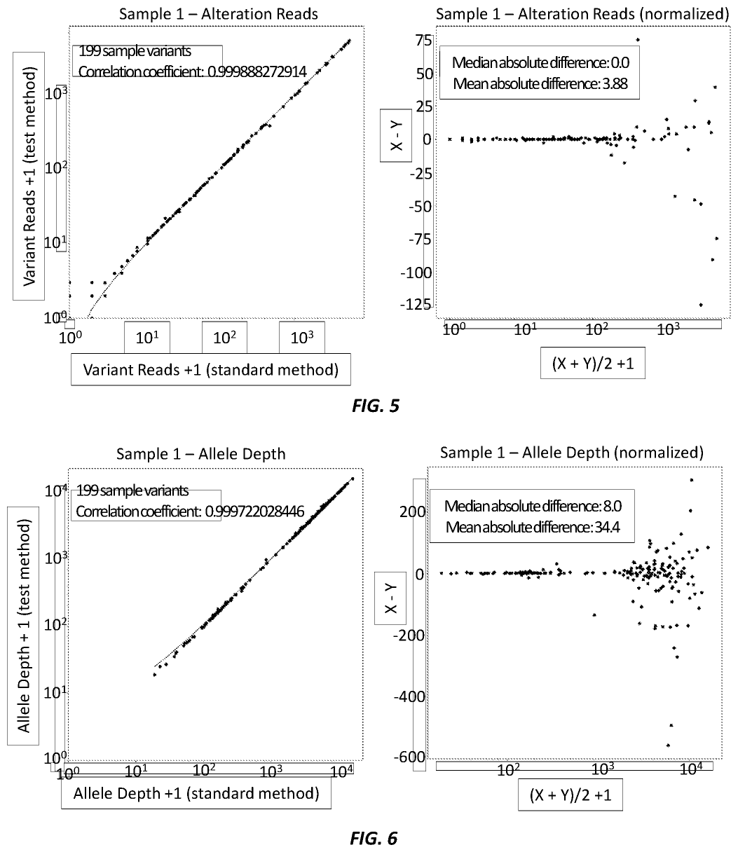

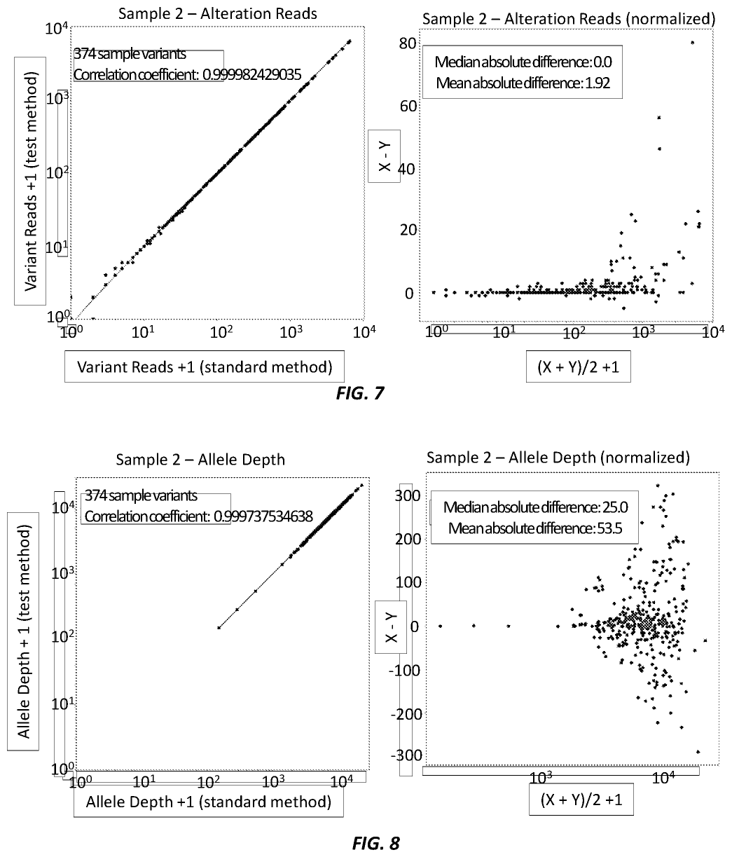

Each sequencing read from Sample 1 and Sample 2 that overlapped a variant locus of a variant in the variant panel was aligned with a reference sequence and a corresponding variant sequence using a Striped Smith-Waterman alignment algorithm to generate a reference match score and a variant match score, respectively. Using the match scores, the reads were labeled as either having the variant, not having the variant, or a inconclusive read. 199 variants from Sample 1 were detected, and 374 variants from Sample 2 were detected. FIG. 5 and FIG. 7 show a plot of the number of variant reads detected by comparing the match scores (y-axis) against the number of variant reads detected using the standard variant calling protocol (x-axis) in log scale (left) and normalized (right) for Sample 1 (FIG. 5) and Sample 2 (FIG. 7). FIG. 6 and FIG. 8 show a plot of the variant allele depth at each variant locus for the sum of sequencing reads labeled as having the variant or not having the variant (i.e., excluding inconclusive reads) (y-axis) against the variant locus depth at each variant locus for the sum of sequencing reads from the initial pool of sequencing reads that overlap with the variant locus (x-axis) in log scale (left) and normalized (right) for Sample 1 (FIG. 6) and Sample 2 (FIG. 8).

### Example 2

Sequencing reads from Sample 1 and Sample 2 were initially obtained using targeted sequencing methods and variants and allele depths called using standard variant calling protocols to generate curated sets of variants from the baseline sample. Variant panels and allele depths were selected for Sample 1 and Sample 2. Variants in the variant panel for Sample 1 ranged from 1 to 22 bases in length (FIG. 3), and variants in in the variant panel for Sample 2 included only variants of a single base length (FIG. 4).

Reference sequences corresponding to each variant in the variant panel (i.e., a reference sequence) and a variant sequence corresponding to each variant in the variant panel (i.e., a variant reference sequence) were generated. The variant or reference base(s) were flanked with 500 bases on each side of the variant locus to generate the corresponding variant sequence and the reference sequence.

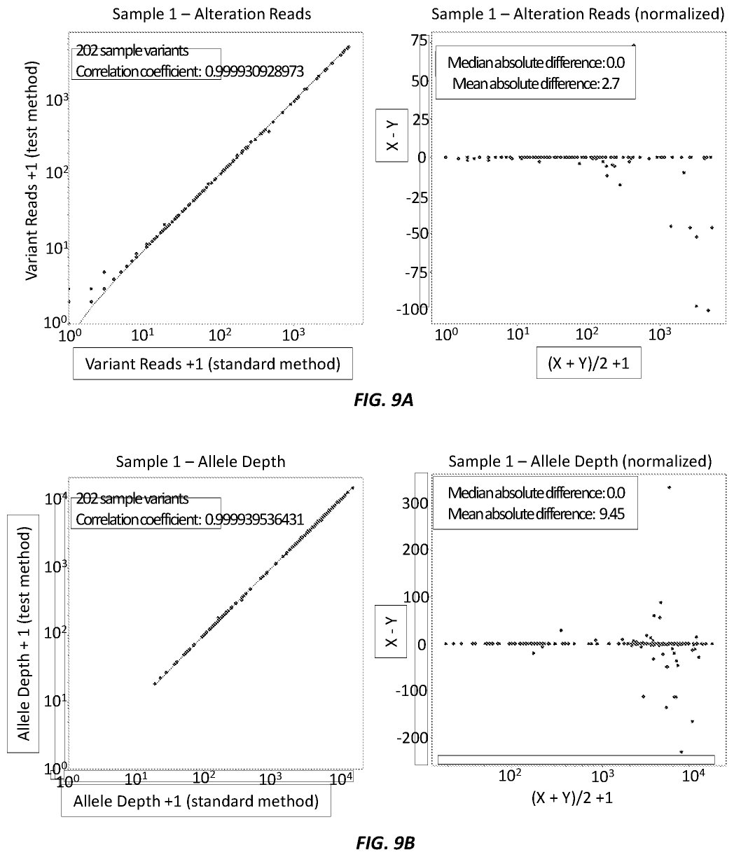

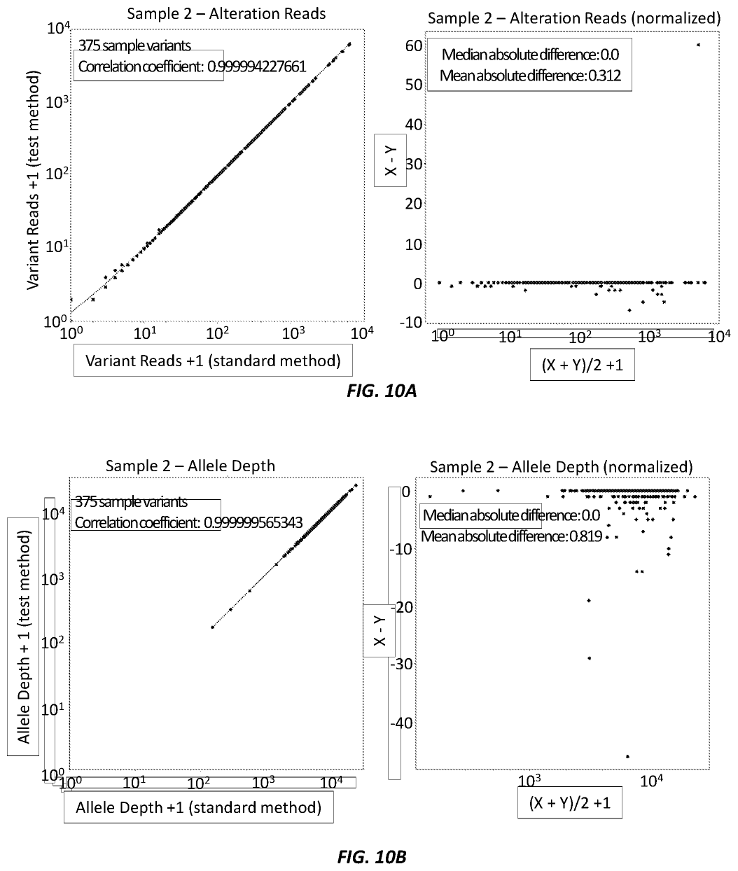

Each sequencing read from Sample 1 and Sample 2 that overlapped a single base of a variant locus of a variant in the variant panel was aligned with a reference sequence and a corresponding variant sequence using a Striped Smith-Waterman alignment algorithm to generate a reference match score and a variant match score, respectively. Using the match scores, the reads were labeled as either having the variant, not having the variant, or an inconclusive read. In some examples, variants from Sample 1 were detected, and 375 variants from Sample 2 were detected. FIG. 9A and FIG. 10A show a plot of the number of variant reads detected by comparing the match scores (y-axis) against the number of variant reads detected using the standard variant calling protocol (x-axis) in log scale (left) and normalized (right) for Sample 1 (FIG. 9A) and Sample 2 (FIG. 10A). FIG. 9B and FIG. 10B show a plot of the variant locus depth at each variant locus for the sum of sequencing reads labeled as having the variant or not having the variant (i.e., excluding inconclusive reads) (y-axis) against the variant locus depth at each variant locus for the sum of sequencing reads from the initial pool of sequencing reads that overlap with the variant locus (x-axis) in log scale (left) and normalized (right) for Sample 1 (FIG. 9B) and Sample 2 (FIG. 10B).

### EXEMPLARY EMBODIMENTS

Among the provided embodiments are:
1. A method of detecting a genetic variant or determining a variant allele frequency in a sample from a subject, comprising:
   providing a plurality of nucleic acid molecules obtained from the sample;
   ligating one or more adapters onto one or more nucleic acid molecules from the plurality of nucleic acid molecules;
   amplifying the one or more ligated nucleic acid molecules from the plurality of nucleic acid molecules;
   capturing the amplified nucleic acid molecules from the amplified nucleic acid molecules;
   sequencing, by a sequencer, the captured nucleic acid molecules to obtain a plurality of sequencing reads that represent the nucleic acid molecules, wherein one or more of the plurality of sequencing reads overlap a variant locus of the genetic variant;
   generating, using one or more processors, a reference match score for each of the one or more sequencing reads by aligning each of the one or more sequencing reads to a reference sequence that does not comprise the genetic variant;
   generating, using the one or more processors, a variant match score for each of the one or more sequencing reads by aligning each sequencing read to a variant sequence that comprises the genetic variant;
   based on the reference match score and the variant match score of a respective sequencing read, labeling, using the one or more processors, each of the one or more sequencing reads as at least one of having the genetic variant, not having the genetic variant, or being an inconclusive read;
   determining, using the one or more processors, a number of sequencing reads labeled as having the genetic variant in the plurality of sequencing reads;
   determining, using the one or more processors, a probability metric based on a variant specific model, the number of sequencing reads labeled as having the genetic variant, and a total number of labeled sequencing reads; and
   identifying, using the one or more processors, the presence of the genetic variant in the sample when the determined probability metric is less than a first threshold.
2. The method of embodiment 1, wherein the variant specific model is locus specific.
3. The method of embodiment 1 and embodiment 2, wherein the first threshold is locus specific and variant specific.
4. The method of embodiments 1-3, wherein the probability metric is a statistical value indicative of a likelihood that the genetic variant is detected due to the presence of the genetic variant in the sample rather than noise.
5. The method of embodiments 1-4, further comprising comparing, using the one or more processors, the determined probability metric to a second threshold, and:
   identifying, by the one or more processors, the absence of the genetic variant in the sample if the determined probability metric is greater than or equal to the second threshold; or
   identifying, by the one or more processors, the presence or absence of the genetic variant in the sample as inconclusive if the determined probability metric is greater than or equal to the first threshold and less than the second threshold.
6. The method of any one of embodiments 1-5, wherein the subject is suspected of or is determined to have cancer.
7. The method of any one of embodiments 1-6, further comprising obtaining the sample from the subject.
8. The method of any one of embodiments 1-7, wherein the sample comprises a tissue biopsy sample, a liquid biopsy sample, or a normal control.
9. The method of embodiment 8, wherein the sample is a liquid biopsy sample and comprises blood, plasma, cerebrospinal fluid, sputum, stool, urine, or saliva.
10. The method of any of embodiment 8 or embodiment 9, wherein the sample is a liquid biopsy sample and comprises cell-free DNA (cfDNA), circulating tumor DNA (ctDNA), or any combination thereof.
11. The method of any of embodiments 1-10, wherein the plurality of nucleic acid molecules comprises a mixture of tumor nucleic acid molecules and non-tumor nucleic acid molecules.
12. The method of embodiment 11, wherein the tumor nucleic acid molecules are derived from a tumor portion of a heterogeneous tissue biopsy sample, and the non-tumor nucleic acid molecules are derived from a normal portion of the heterogeneous tissue biopsy sample.
13. The method of embodiment 11, wherein the sample comprises a liquid biopsy sample, and wherein the tumor nucleic acid molecules are derived from a circulating tumor DNA (ctDNA) fraction of the liquid biopsy sample, and the non-tumor nucleic acid molecules are derived from a non-tumor, cell-free DNA (cfDNA) fraction of the liquid biopsy sample.
14. The method of any one of embodiments 1-13, wherein the one or more adapters comprise amplification primers, flow cell adaptor sequences, substrate adapter sequences, or sample index sequences.
15. The method of any one of embodiments 1-14, wherein the captured nucleic acid molecules are captured from the amplified nucleic acid molecules by hybridization to one or more bait molecules.
16. The method of embodiment 15, wherein the one or more bait molecules comprise one or more nucleic acid molecules, each comprising a region that is complementary to a region of a captured nucleic acid molecule.
17. The method of any one of embodiments 1-16, wherein amplifying nucleic acid molecules comprises performing a polymerase chain reaction (PCR) amplification technique, a non-PCR amplification technique, or an isothermal amplification technique.
18. The method of any one of embodiments 1-17, wherein the sequencing comprises use of a next generation sequencing (NGS) technique, whole genome sequencing (WGS), whole exome sequencing, targeted sequencing, direct sequencing, or Sanger sequencing technique.
19. The method of any one of embodiments 1-18, wherein the sequencer comprises a next generation sequencer.
20. The method of any one of embodiments 1-19, further comprising generating, by the one or more processors, a report indicating a report indicating the presence or absence of the genetic variant.
21. The method of embodiment 20, comprising transmitting the report to a healthcare provider.
22. The method of embodiment 20, wherein the report is transmitted via a computer network or a peer-to-peer connection.
23. A method of detecting a genetic variant in a sample from a subject, comprising:
   obtaining a plurality of sequencing reads associated with the sample, wherein one or more of the plurality of sequencing reads that overlap a variant locus associated with the genetic variant;
   generating, by one or more processors, a reference match score for each of the plurality of sequencing reads by aligning each of the one or more sequencing reads to a reference sequence that does not comprise the genetic variant;
   generating, by the one or more processors, a variant match score for each of the plurality of sequencing reads by aligning each sequencing read to a variant sequence that comprises the genetic variant;
   labeling, by the one or more processors, each of the plurality of sequencing reads as at least one of having the genetic variant, not having the genetic variant, or being an inconclusive read based on the reference match score and the variant match score of the respective sequencing read;
   determining, by the one or more processors, a number of sequencing reads labeled as having the genetic variant in the plurality of sequencing reads;
   determining, by the one or more processors, a probability metric based on a variant specific model, the number of sequencing reads labeled as having the genetic variant, and a total number of labeled sequencing reads; and
   identifying, by the one or more processors, the presence of the genetic variant in the sample when the determined probability metric is less than a first threshold.
24. The method of embodiment 23, wherein the variant specific model is locus specific.
25. The method of any of embodiment 23 and embodiment 24, wherein the first threshold is locus specific and variant specific.
26. The method of any one of embodiments 23-25, wherein the probability metric corresponds to a probability that the genetic variant is detected due to the presence of the genetic variant in the sample rather than noise.
27. The method of any one of embodiments 23-26, further comprising comparing, using the one or more processors, the determined probability metric to a second threshold, and:
   identifying, by the one or more processors, the absence of the genetic variant in the sample if the determined probability metric is greater than or equal to the second threshold; or
   identifying, by the one or more processors, the presence or absence of the genetic variant in the sample as inconclusive if the determined probability metric is greater than or equal to the first threshold and less than the second threshold.
28. The method of any one of embodiments 23-27, wherein the variant specific model is generated by:
   fitting, using the one or more processors, a probability distribution based on a determined metric and a total number of labeled sequencing reads from a wild-type sample.
29. The method of embodiment 28, wherein the probability distribution is a binomial distribution.
30. The method of any one of embodiments 23-29, wherein the probability metric is determined from the number of sequencing reads labeled as having the genetic variant and a second number, wherein the second number is the total number of labeled sequencing reads minus a number of sequencing reads labeled as being inconclusive reads.
31. The method of any one of embodiments 23-30, wherein the variant specific model is associated with one or more functions related to one of more sources of noise in a plurality of sequencing reads that overlap the variant locus.
32. The method of embodiment 31, wherein the one or more sources of noise comprise sample preparation errors, amplification bias errors, sequencing errors, alignment errors, or any combination thereof.
33. The method of any one of embodiments 23-32, wherein the variant specific model is associated with one or more functions that have been fitted to data for a plurality of sequencing reads that overlap the variant locus.
34. The method of embodiment 33, wherein the one or more functions comprise one or more of uniform distribution functions, binomial distribution functions, Poisson distribution functions, negative binomial distribution functions, normal distribution functions, log-normal distribution functions, Cauchy-Lorentz distribution functions, log-logistic distribution functions, exponential distribution functions, gamma distribution functions, hypergeometric distribution functions, or any combination thereof.
35. The method of any one of embodiments 23-34, wherein a sequencing read is labeled as having the genetic variant if the reference match score and the variant match score indicate that the sequencing read more closely matches the variant sequence than the reference sequence.
36. The method of any one of embodiments 23-35, wherein a sequencing read is labeled as not having the genetic variant if the reference match score and the variant match score indicate that the sequencing read more closely matches the reference sequence than the variant sequence.
37. The method of any one of embodiments 23-36, wherein a sequencing read is labeled as the inconclusive read if the reference match score and the variant match score are equal.
38. The method of any one of embodiments 23-37, wherein the first threshold is determined empirically using the variant specific model.
39. The method of any one of embodiments 23-38, wherein at least one of the first threshold or the second threshold is determined empirically using clinical trial outcomes.
40. The method of any one of embodiments 23-39, wherein the first threshold is determined using a Kaplan-Meier estimator and data associated with samples from a plurality of subjects.
41. The method of embodiment 39, wherein the second threshold is determined empirically using the variant specific model, and is set to a value that corresponds to a specified confidence level that a sequencing read labeled as not containing the genetic variant is correct.
42. The method of any one of embodiments 23-41, wherein the reference sequence and the variant sequence comprise the variant locus, a 5′ flanking region, and a 3′ flanking region.
43. The method of embodiment 42, wherein 5′ flanking region and 3′ flanking region are each about 5 bases in length to about 5000 bases in length.
44. The method of any one of embodiments 23-43, comprising generating from the sample, the variant sequence.
45. The method of embodiment 44, wherein generating the variant sequence comprises:
   providing a plurality of nucleic acid molecules obtained from the sample;
   ligating one or more adapters onto one or more nucleic acid molecules from the plurality of nucleic acid molecules;
   amplifying the one or more ligated nucleic acid molecules from the plurality of nucleic acid molecules;
   capturing the amplified nucleic acid molecules from the amplified nucleic acid molecules; and
   sequencing, by a sequencer, the captured nucleic acid molecules to obtain a plurality of sequencing reads that represent the nucleic acid molecules, wherein one or more of the plurality of sequencing reads overlap a variant locus of the genetic variant.
46. The method of any one of embodiments 23-45, wherein the reference sequence and the variant sequence are substantially identical except for the genetic variant.
47. The method of any one of embodiments 23-46, comprising determining a variant allele frequency for the genetic variant using the number of sequencing reads labeled as having the genetic variant and the number of sequencing reads labeled as not having the genetic variant.
48. The method of any one of embodiments 23-47, comprising:
   labeling sequencing reads associated with the sample for a second genetic variant selected from the one or more variants;
   determining a probability metric using a second variant specific model, the number of sequencing reads labeled as having the second genetic variant and a total number of labeled sequencing reads for the second genetic variant; and
   comparing the determined probability metric for the second genetic variant to a corresponding third threshold, wherein if the determined probability metric for the second genetic variant is less than the third threshold, the presence of the second genetic variant in the sample is identified.
49. The method of embodiment 48, wherein the second genetic variant is associated with a second variant locus selected from the one or more variants.
50. The method of embodiment 49, further comprising:
   comparing the determined probability metric for the second genetic variant to a fourth threshold;
   when the determined probability metric is greater than or equal to the fourth threshold, identifying the absence of the second genetic variant in the sample; and
   when the determined probability metric is greater than or equal to the third threshold and less than the fourth threshold, the presence or absence of the second genetic variant in the sample is inconclusive.
51. The method of any one of embodiments 23-50, comprising determining a disease status for the subject.
52. The method of embodiment 51, wherein the disease status is a value proportional to a percentage of circulating-tumor DNA (ctDNA) compared to total cell-free DNA (cfDNA) in the sample.
53. The method of embodiment 52, wherein the disease status is a maximum somatic allele fraction of cfDNA.
54. The method of embodiment 52, wherein the disease status comprises a qualitative factor indicating recurrence of a cancer in the subject, the presence of a cancer resistant to a treatment modality in the subject, or the presence of a cancer that can be treated with a particular treatment modality.
55. The method of any one of embodiments 23-54, wherein the sample comprises cfDNA.
56. The method of any one of embodiments 23-55, wherein the reference match score and the variant match score are determined using a sequence alignment algorithm.
57. The method of embodiment 56, wherein the sequence alignment algorithm is at least one of a Smith-Waterman alignment algorithm, a Striped Smith-Waterman alignment algorithm, or a Needleman-Wunsch alignment algorithm.
58. The method of any one of embodiments 23-57, wherein the genetic variant comprises a single nucleotide variant (SNV), a multiple nucleotide variant (MNV), an indel, or a rearrangement junction.
59. The method of any one of embodiments 23 to 58, wherein the variant panel is determined by sequencing nucleic acid molecules in a previous sample obtained from the subject, and identifying one or more genetic variants.
60. The method of embodiment 59, wherein the subject received an intervening treatment for a disease between the previous sample being obtained and the sample being obtained.
61. The method of embodiment 60, wherein the disease is cancer.
62. The method of embodiment 59 or embodiment 60, further comprising adjusting the treatment based on a difference between a disease status for the subject determined using the sample and a previous disease status for the subject based on the previous sample.
63. The method of any one of embodiments 23-62, comprising generating the one or more sequencing reads by sequencing nucleic acid molecules in the sample.
64. The method of any one of embodiments 23-63, wherein the variant is a somatic mutation.
65. The method of any one of embodiments 23-64, wherein the variant is a germline mutation.
66. The method of any of embodiments 23-65, further comprising: determining, identifying, or applying the presence of the genetic variant of the sample as a diagnostic value associated with the sample.
67. The method of any of embodiments 23-66, further comprising: generating a genomic profile for the subject based on the presence of the genetic variant.
68. The method of embodiment 67, further comprising: selecting an anti-cancer agent, administering an anti-cancer agent or applying an anti-cancer treatment to the subject based on the generated genomic profile.
69. The method of any of embodiments 23-68, wherein the presence of the genetic variant of the sample is used in generating a genomic profile for the subject.
70. The method of any of embodiments 23-69, wherein the presence of the genetic variant of the sample is used in making suggested treatment decisions for the subject.
71. The method of any of embodiments 23-70, wherein the presence of the genetic variant of the sample is used in applying or administering a treatment to the subject.
72. A method for detecting a disease state in a sample from a subject, comprising:
   sequencing nucleic acid molecules in the sample acquired from the subject to generate a plurality of sequencing reads; and
   detecting a genetic variant of determining a variant allele frequency in the sample according to the method of any one of embodiments 1 to 71.
73. A method of monitoring disease progression or recurrence, comprising:
   sequencing nucleic acid molecules in a first sample acquired from a subject with a disease to generate a first set of sequencing reads;
   generating a personalized variant panel for the subject;
   sequencing nucleic acid molecules in a second sample acquired from the subject at a later time point than the first sample to generate a second set of sequencing reads; and
   detecting, using the second set of sequencing reads, a genetic variant or determining, using the second set of sequencing reads, a variant allele frequency according to the method of any one of embodiments 1 to 71.
74. The method of embodiment 73, comprising administering a disease therapy to the subject after the first sample is acquired from the subject and before the second sample is acquired from the subject.
75. The method of embodiment 73 or 74, comprising:
   determining a first disease status based on a number of sequencing reads in the first set of sequencing reads labeled as having a genetic variant from the variant panel; and
   determining a second disease status based on a number of sequencing reads in the second set of sequencing reads labeled as having the genetic variant from the variant panel.
76. The method of embodiment 75, further comprising determining disease progression by comparing the first disease status and the second disease status.
77. The method of embodiment 76, comprising:
   administering a disease therapy to the subject after the first sample is acquired from the subject and before the second sample is acquired from the subject; and
   adjusting the disease therapy based on the determined disease progression.
78. A method of treating a subject with a disease, comprising:
   acquiring a first sample from the subject;
   sequencing nucleic acid molecules in a first sample to generate a first set of sequencing reads;
   determining a first disease status using the first set of sequencing reads;
   generating a personalized variant panel for the subject;
   administering a disease therapy to the subject;
   acquiring a second sample from the subject after the disease therapy has been administered to the subject;
   sequencing nucleic acid molecules in the second sample to generate a second set of sequencing reads;
   detecting, using the second set of sequencing reads, a genetic variant or determining, using the second set of sequencing reads, a variant allele frequency according to the method of any one of embodiments 1 to 71;
   determining a second disease status based on the second set of sequencing reads;
   determining disease progression by comparing the first disease status and the second disease status;
   adjusting the disease therapy administered to subject based on the disease progression; and
   administering the adjusted disease therapy to the subject.
79. The method of embodiment 78, wherein the disease is cancer.
80. The method of any one of embodiments 1-79, wherein the sample is derived from a liquid biopsy sample from the subject.
81. The method of any one of embodiments 1-80, wherein the sample is derived from a solid tissue sample, liquid tissue sample, or hematological sample, from the subject.
82. The method of any one of embodiments 23-81, further comprising sequencing nucleic acid molecules extracted from the sample to generate the plurality of sequencing reads.
83. The method of any one of embodiments 23-82, comprising generating or updating a report comprising (1) identifying information for the subject, and (2) a call for the presence or absence of the genetic variant, or a call for the variant allele frequency for the genetic variant.
84. The method of embodiment 83, further comprising transmitting the report to the subject or a healthcare provider for the subject.
85. An apparatus, comprising:
   one or more processors;
   a memory; and
   one or more programs, wherein the one or more programs are stored in the memory and configured to be executed by the one or more processors, the one or more programs including instructions for:
   selecting a genetic variant at a variant locus from one or more variants;
   obtaining a plurality of sequencing reads associated with a sample that overlap the variant locus;
   generating a reference match score for each of the plurality of sequencing reads by aligning each sequencing read to a reference sequence that does not comprise the genetic variant;
   generating a variant match score for each of the plurality of sequencing reads by aligning each sequencing read to a variant sequence that comprises the genetic variant;
   labeling each of the one or more sequencing reads as at least one of having the genetic variant, as not having the genetic variant, or as being an inconclusive read based on the reference match score and the variant match score of the respective sequencing read;
   determining a number of sequencing reads labeled as having the genetic variant;
   determining a probability metric based on a variant specific model and a total number of labeled sequencing reads; and
   identifying, using the one or more processors, the presence of the genetic variant in the sample if the determined probability metric is less than a first threshold.
86. The apparatus of embodiment 85, wherein the variant specific model is locus specific.
87. The apparatus of any of embodiment 85 and embodiment 86, wherein the first threshold is locus specific and variant specific.
88. The apparatus of any one of embodiments 85-87, wherein the probability metric is a statistical value indicative of a likelihood that the genetic variant is detected due to the presence of the genetic variant in the sample rather than noise.
89. The apparatus of any one of embodiments 85-88, the one or more programs further including instructions for:
   comparing, using the one or more processors, the determined probability metric to a second threshold, and:
   identifying, by the one or more processors, the absence of the genetic variant in the sample if the determined probability metric is greater than or equal to the second threshold; or
   identifying, by the one or more processors, the presence or absence of the genetic variant in the sample as inconclusive if the determined probability metric is greater than or equal to the first threshold and less than the second threshold.
90. The apparatus of any one of embodiments 85-89, wherein the variant specific model is generated by:
   fitting, using the one or more processors, a probability distribution based on a determined metric and a total number of labeled sequencing reads from a wild-type sample.
91. The apparatus of embodiment 90, wherein the probability distribution is a binomial distribution.
92. The apparatus of any one of embodiments 85-91, wherein the probability metric is determined from the number of sequencing reads labeled as having the genetic variant and a second number, wherein the second number is the total number of labeled sequencing reads minus a number of sequencing reads labeled as being inconclusive reads.
93. The apparatus of any one of embodiments 85-92, wherein the variant specific model is associated with one or more functions related to one of more sources of noise in a plurality of sequencing reads that overlap the variant locus.
94. The apparatus of embodiment 93, wherein the one or more sources of noise comprise sample preparation errors, amplification bias errors, sequencing errors, alignment errors, or any combination thereof.
95. The apparatus of any one of embodiments 85-94, wherein the variant specific model is associated with one or more functions that have been fitted to data for a plurality of sequencing reads that overlap the variant locus.
96. The apparatus of embodiment 95, wherein the one or more functions comprise one or more of uniform distribution functions, binomial distribution functions, Poisson distribution functions, negative binomial distribution functions, normal distribution functions, log-normal distribution functions, Cauchy-Lorentz distribution functions, log-logistic distribution functions, exponential distribution functions, gamma distribution functions, hypergeometric distribution functions, or any combination thereof.
97. The apparatus of any one of embodiments 85-96, wherein a sequencing read is labeled as having the genetic variant if the reference match score and the variant match score indicate that the sequencing read more closely matches the variant sequence than the reference sequence.
98. The apparatus of any one of embodiments 85-97, wherein a sequencing read is labeled as not having the genetic variant if the reference match score and the variant match score indicate that the sequencing read more closely matches the reference sequence than the variant sequence.
99. The apparatus of any one of embodiments 85-98, wherein a sequencing read is labeled as the inconclusive read if the reference match score and the variant match score are equal.
100. The apparatus of any one of embodiments 85-99, wherein the first threshold is determined empirically using the variant specific model.
101. The apparatus of any one of embodiments 85-100, wherein at least one of the first threshold or the second threshold is determined empirically using clinical trial outcomes.
102. The apparatus of any one of embodiments 85-101, wherein the first threshold is determined using a Kaplan-Meier estimator and data associated with samples from a plurality of subjects.
103. The apparatus of embodiment 102, wherein the second threshold is determined empirically using the variant specific model, and is set to a value that corresponds to a specified confidence level that a sequencing read labeled as not containing the genetic variant is correct.
104. The apparatus of any one of embodiments 85-103, wherein the reference sequence and the variant sequence comprise the variant locus, a 5′ flanking region, and a 3′ flanking region.
105. The apparatus of embodiment 104, wherein 5′ flanking region and 3′ flanking region are each about 5 bases in length to about 5000 bases in length.
106. The apparatus of any one of embodiments 85-105, wherein the one or more programs further include instructions for generating from the sample, the variant sequence.
107. The apparatus of embodiment 106, wherein generating the variant sequence comprises:
   providing a plurality of nucleic acid molecules obtained from the sample;
   ligating one or more adapters onto one or more nucleic acid molecules from the plurality of nucleic acid molecules;
   amplifying the one or more ligated nucleic acid molecules from the plurality of nucleic acid molecules;
   capturing the amplified nucleic acid molecules from the amplified nucleic acid molecules; and
   sequencing, by a sequencer, the captured nucleic acid molecules to obtain a plurality of sequencing reads that represent the nucleic acid molecules, wherein one or more of the plurality of sequencing reads overlap a variant locus of the genetic variant.
108. The apparatus of any one of embodiments 85-107, wherein the reference sequence and the variant sequence are substantially identical except for the genetic variant.
109. The apparatus of any one of embodiments 85-108, wherein the one or more programs further include instructions for determining a variant allele frequency for the genetic variant using the number of sequencing reads labeled as having the genetic variant and the number of sequencing reads labeled as not having the genetic variant.
110. The apparatus of any one of embodiments 85-109, wherein the one or more programs further include instructions for:
   labeling sequencing reads associated with the sample for a second genetic variant selected from the one or more variants;
   determining a probability metric using a second variant specific model, the number of sequencing reads labeled as having the second genetic variant and a total number of labeled sequencing reads for the second genetic variant; and
   comparing the determined probability metric for the second genetic variant to a corresponding third threshold, wherein if the determined probability metric for the second genetic variant is less than the third threshold, the presence of the second genetic variant in the sample is identified.
111. The apparatus of embodiment 110, wherein the second genetic variant is associated with a second variant locus selected from the one or more variants.
112. The apparatus of embodiment 111, the one or more programs further including instructions for: comparing the determined probability metric for the second genetic variant to a fourth threshold;
   when the determined probability metric is greater than or equal to the fourth threshold, identifying the absence of the second genetic variant in the sample; and
   when the determined probability metric is greater than or equal to the third threshold and less than the fourth threshold, the presence or absence of the second genetic variant in the sample is inconclusive.
113. The apparatus of any one of embodiments 85-112, wherein the one or more programs further include instructions for determining a disease status for the subject.
114. The apparatus of embodiment 113, wherein the disease status is a value proportional to a percentage of circulating-tumor DNA (ctDNA) compared to total cell-free DNA (cfDNA) in the sample.
115. The apparatus of embodiment 114, wherein the disease status is a maximum somatic allele fraction of cfDNA.
116. The apparatus of embodiment 114, wherein the disease status comprises a qualitative factor indicating recurrence of a cancer in the subject, the presence of a cancer resistant to a treatment modality in the subject, or the presence of a cancer that can be treated with a particular treatment modality.
117. The apparatus of any one of embodiments 85-116, wherein the sample comprises cfDNA.
118. The apparatus of any one of embodiments 85-117, wherein the reference match score and the variant match score are determined using a sequence alignment algorithm.
119. The apparatus of embodiment 118, wherein the sequence alignment algorithm is at least one of a Smith-Waterman alignment algorithm, a Striped Smith-Waterman alignment algorithm, or a Needleman-Wunsch alignment algorithm.
120. The apparatus of any one of embodiments 85-119, wherein the genetic variant comprises a single nucleotide variant (SNV), a multiple nucleotide variant (MNV), an indel, or a rearrangement junction.
121. The apparatus of any one of embodiments 85 to 120, wherein the variant panel is determined by sequencing nucleic acid molecules in a previous sample obtained from the subject, and identifying one or more genetic variants.
122. The apparatus of embodiment 121, wherein the subject received an intervening treatment for a disease between the previous sample being obtained and the sample being obtained.
123. The apparatus of embodiment 122, wherein the disease is cancer.
124. The apparatus of embodiment 121 or embodiment 122, the one or more programs further including instructions for: adjusting the treatment based on a difference between a disease status for the subject determined using the sample and a previous disease status for the subject based on the previous sample.
125. The apparatus of any one of embodiments 85-124, wherein the one or more programs further include instructions for generating the one or more sequencing reads by sequencing nucleic acid molecules in the sample.
126. The apparatus of any one of embodiments 85-125, wherein the variant is a somatic mutation.
127. The apparatus of any one of embodiments 85-126, wherein the variant is a germline mutation.
128. The apparatus of any of embodiments 85-127, the one or more programs further including instructions for: determining, identifying, or applying the presence of the genetic variant of the sample as a diagnostic value associated with the sample.
129. The apparatus of any of embodiments 85-128, the one or more programs further including instructions for: generating a genomic profile for the subject based on the presence of the genetic variant.
130. The apparatus of embodiment 129, the one or more programs further including instructions for: administering an anti-cancer agent or applying an anti-cancer treatment to the subject based on the generated genomic profile.
131. The apparatus of any of embodiments 85-130, wherein the presence of the genetic variant of the sample is used in generating a genomic profile for the subject.
132. The apparatus of any of embodiments 85-131, wherein the presence of the genetic variant of the sample is used in making suggested treatment decisions for the subject.
133. The apparatus of any of embodiments 85-132, wherein the presence of the genetic variant of the sample is used in applying or administering a treatment to the subject.
134. A non-transitory computer-readable storage medium storing one or more programs, the one or more programs comprising instructions, the instructions when executed by one or more processors of an electronic device, cause the electronic device to:
   select a genetic variant at a variant locus from one or more variants;
   obtain a plurality of sequencing reads associated with a sample that overlaps the variant locus;
   generate a reference match score for each of the plurality of sequencing reads by aligning each sequencing read to a reference sequence that does not comprise the genetic variant;
   generate a variant match score for each of the plurality of sequencing reads by aligning each sequencing read to a variant sequence that comprises the genetic variant;
   label each of the plurality of sequencing reads as at least one of having the genetic variant, as not having the genetic variant, or as being an inconclusive read based on the reference match score and the variant match score of the respective sequencing read;
   determine a number of sequencing reads labeled as having the genetic variant;
   determine a probability metric based on a variant specific model and a total number of labeled sequencing reads; and
   identify the presence of the genetic variant in the sample if the determined probability metric is less than a first threshold.
135. The non-transitory computer-readable storage medium of embodiment 134, wherein the variant specific model is locus specific.
136. The non-transitory computer-readable storage medium of any of embodiment 134 and embodiment 135, wherein the first threshold is locus specific and variant specific.
137. The non-transitory computer-readable storage medium of any one of embodiments 134-136, wherein the probability metric is a statistical value indicative of a likelihood that the genetic variant is detected due to the presence of the genetic variant in the sample rather than noise.
138. The non-transitory computer-readable storage medium of any one of embodiments 134-137, the one or more programs further including instructions for:
   comparing, using the one or more processors, the determined probability metric to a second threshold, and:
   identifying, by the one or more processors, the absence of the genetic variant in the sample if the determined probability metric is greater than or equal to the second threshold; or
   identifying, by the one or more processors, the presence or absence of the genetic variant in the sample as inconclusive if the determined probability metric is greater than or equal to the first threshold and less than the second threshold.
139. The non-transitory computer-readable storage medium of any one of embodiments 134-138, wherein the variant specific model is generated by:
   fitting, using the one or more processors, a probability distribution based on a determined metric and a total number of labeled sequencing reads from a wild-type sample.
140. The non-transitory computer-readable storage medium of embodiment 139, wherein the probability distribution is a binomial distribution.
141. The non-transitory computer-readable storage medium of any one of embodiments 134-140, wherein the probability metric is determined from the number of sequencing reads labeled as having the genetic variant and a second number, wherein the second number is the total number of labeled sequencing reads minus a number of sequencing reads labeled as being inconclusive reads.
142. The non-transitory computer-readable storage medium of any one of embodiments 134-141, wherein the variant specific model is associated with one or more functions related to one of more sources of noise in a plurality of sequencing reads that overlap the variant locus.
143. The non-transitory computer-readable storage medium of embodiment 142, wherein the one or more sources of noise comprise sample preparation errors, amplification bias errors, sequencing errors, alignment errors, or any combination thereof.
144. The non-transitory computer-readable storage medium of any one of embodiments 134-143, wherein the variant specific model is associated with one or more functions that have been fitted to data for a plurality of sequencing reads that overlap the variant locus.
145. The non-transitory computer-readable storage medium of embodiment 144, wherein the one or more functions comprise one or more of uniform distribution functions, binomial distribution functions, Poisson distribution functions, negative binomial distribution functions, normal distribution functions, log-normal distribution functions, Cauchy-Lorentz distribution functions, log-logistic distribution functions, exponential distribution functions, gamma distribution functions, hypergeometric distribution functions, or any combination thereof.
146. The non-transitory computer-readable storage medium of any one of embodiments 134-145, wherein a sequencing read is labeled as having the genetic variant if the reference match score and the variant match score indicate that the sequencing read more closely matches the variant sequence than the reference sequence.
147. The non-transitory computer-readable storage medium of any one of embodiments 134-146, wherein a sequencing read is labeled as not having the genetic variant if the reference match score and the variant match score indicate that the sequencing read more closely matches the reference sequence than the variant sequence.
148. The non-transitory computer-readable storage medium of any one of embodiments 134-147, wherein a sequencing read is labeled as the inconclusive read if the reference match score and the variant match score are equal.
149. The non-transitory computer-readable storage medium of any one of embodiments 134-148, wherein the first threshold is determined empirically using the variant specific model.
150. The non-transitory computer-readable storage medium of any one of embodiments 134-149, wherein at least one of the first threshold or the second threshold is determined empirically using clinical trial outcomes.
151. The non-transitory computer-readable storage medium of any one of embodiments 134-150, wherein the first threshold is determined using a Kaplan-Meier estimator and data associated with samples from a plurality of subjects.
152. The non-transitory computer-readable storage medium of embodiment 150, wherein the second threshold is determined empirically using the variant specific model, and is set to a value that corresponds to a specified confidence level that a sequencing read labeled as not containing the genetic variant is correct.
153. The non-transitory computer-readable storage medium of any one of embodiments 134-152, wherein the reference sequence and the variant sequence comprise the variant locus, a 5′ flanking region, and a 3′ flanking region.
154. The non-transitory computer-readable storage medium of embodiment 153, wherein the 5′ flanking region and 3′ flanking region are each about 5 bases in length to about 5000 bases in length.
155. The non-transitory computer-readable storage medium of any one of embodiments 134-154, the one or more programs further comprising instructions for generating from the sample, the variant sequence.
156. The non-transitory computer-readable storage medium of embodiment 155, wherein generating the variant sequence comprises:
   providing a plurality of nucleic acid molecules obtained from the sample;
   ligating one or more adapters onto one or more nucleic acid molecules from the plurality of nucleic acid molecules;
   amplifying the one or more ligated nucleic acid molecules from the plurality of nucleic acid molecules;
   capturing the amplified nucleic acid molecules from the amplified nucleic acid molecules; and
   sequencing, by a sequencer, the captured nucleic acid molecules to obtain a plurality of sequencing reads that represent the nucleic acid molecules, wherein one or more of the plurality of sequencing reads overlap a variant locus of the genetic variant.
157. The non-transitory computer-readable storage medium of any one of embodiments 134-156, wherein the reference sequence and the variant sequence are substantially identical except for the genetic variant.
158. The non-transitory computer-readable storage medium of any one of embodiments 134-157, the one or more programs further comprising instructions for determining a variant allele frequency for the genetic variant using the number of sequencing reads labeled as having the genetic variant and the number of sequencing reads labeled as not having the genetic variant.
159. The non-transitory computer-readable storage medium of any one of embodiments 134-158, the one or more programs further comprising instructions for:
   labeling sequencing reads associated with the sample for a second genetic variant selected from the one or more variants;
   determining a probability metric using a second variant specific model, the number of sequencing reads labeled as having the second genetic variant and a total number of labeled sequencing reads for the second genetic variant; and
   comparing the determined probability metric for the second genetic variant to a corresponding third threshold, wherein if the determined probability metric for the second genetic variant is less than the third threshold, the presence of the second genetic variant in the sample is identified.
160. The non-transitory computer-readable storage medium of embodiment 159, wherein the second genetic variant is associated with a second variant locus selected from the one or more variants.
161. The non-transitory computer-readable storage medium of embodiment 160, the one or more programs further comprising instructions for:
   comparing the determined probability metric for the second genetic variant to a fourth threshold;
   when the determined probability metric is greater than or equal to the fourth threshold, identifying the absence of the second genetic variant in the sample; and
   when the determined probability metric is greater than or equal to the third threshold and less than the fourth threshold, the presence or absence of the second genetic variant in the sample is inconclusive.
162. The non-transitory computer-readable storage medium of any one of embodiments 134-161, the one or more programs further comprising instructions for determining a disease status for the subject.
163. The non-transitory computer-readable storage medium of embodiment 162, wherein the disease status is a value proportional to a percentage of circulating-tumor DNA (ctDNA) compared to total cell-free DNA (cfDNA) in the sample.
164. The non-transitory computer-readable storage medium of embodiment 163, wherein the disease status is a maximum somatic allele fraction of cfDNA.
165. The non-transitory computer-readable storage medium of embodiment 163, wherein the disease status comprises a qualitative factor indicating recurrence of a cancer in the subject, the presence of a cancer resistant to a treatment modality in the subject, or the presence of a cancer that can be treated with a particular treatment modality.
166. The non-transitory computer-readable storage medium of any one of embodiments 134-165, wherein the sample comprises cfDNA.
167. The non-transitory computer-readable storage medium of any one of embodiments 134-166, wherein the reference match score and the variant match score are determined using a sequence alignment algorithm.
168. The non-transitory computer-readable storage medium of embodiment 167, wherein the sequence alignment algorithm is at least one of a Smith-Waterman alignment algorithm, a Striped Smith-Waterman alignment algorithm, or a Needleman-Wunsch alignment algorithm.
169. The non-transitory computer-readable storage medium of any one of embodiments 134-168, wherein the genetic variant comprises a single nucleotide variant (SNV), a multiple nucleotide variant (MNV), an indel, or a rearrangement junction.
170. The non-transitory computer-readable storage medium of any one of embodiments 134 to 169, wherein the variant panel is determined by sequencing nucleic acid molecules in a previous sample obtained from the subject, and identifying one or more genetic variants.
171. The non-transitory computer-readable storage medium of embodiment 170, wherein the subject received an intervening treatment for a disease between the previous sample being obtained and the sample being obtained.
172. The non-transitory computer-readable storage medium of embodiment 171, wherein the disease is cancer.
173. The non-transitory computer-readable storage medium of embodiment 170 or embodiment 171, the one or more programs further comprising instructions for adjusting the treatment based on a difference between a disease status for the subject determined using the sample and a previous disease status for the subject based on the previous sample.
174. The non-transitory computer-readable storage medium of any one of embodiments 134-173, the one or more programs further comprising instructions for generating the one or more sequencing reads by sequencing nucleic acid molecules in the sample.
175. The non-transitory computer-readable storage medium of any one of embodiments 134-174, wherein the variant is a somatic mutation.
176. The non-transitory computer-readable storage medium of any one of embodiments 134-175, wherein the variant is a germline mutation.
177. The non-transitory computer-readable storage medium of any of embodiments 134-176, the one or more programs further comprising instructions for determining, identifying, or applying the presence of the genetic variant of the sample as a diagnostic value associated with the sample.
178. The non-transitory computer-readable storage medium of any of embodiments 134-177, the one or more programs further comprising instructions for generating a genomic profile for the subject based on the presence of the genetic variant.
179. The non-transitory computer-readable storage medium of embodiment 178, the one or more programs further comprising instructions for administering an anti-cancer agent or applying an anti-cancer treatment to the subject based on the generated genomic profile.
180. The non-transitory computer-readable storage medium of any of embodiments 134-179, wherein the presence of the genetic variant of the sample is used in generating a genomic profile for the subject.
181. The non-transitory computer-readable storage medium of any of embodiments 134-180, wherein the presence of the genetic variant of the sample is used in making suggested treatment decisions for the subject.
182. The non-transitory computer-readable storage medium of any of embodiments 134-181, wherein the presence of the genetic variant of the sample is used in applying or administering a treatment to the subject.
183. A computer system comprising:
   a processor; and
   a memory communicatively coupled to the processor, configured to store instructions that, when executed by the processor cause the processor to perform the method of any one of embodiments 1-86.
184. The method of any of embodiments 1-22, wherein the plurality of sequencing reads comprises between 100 and 3,000 loci, between 200 and 2,800 loci, between 300 and 2,600 loci, between 400 and 2,400 loci, between 500 and 2,200 loci, between 600 and 2,000 loci, between 700 and 1,800 loci, between 800 and 1,600 loci, between 900 and 1,400 loci, between 1,000 and 1,200 loci, between 400 and 1,000 loci, between 400 and 1,200 loci, between 400 and 1,400 loci, between 400 and 1,600 loci, between 400 and 1,800 loci, between 400 and 2,000 loci, between 400 and 2,200 loci, between 400 and 2,400 loci, between 400 and 2,600 loci, between 400 and 2,800 loci, between 400, and 3,000 loci, between 600 and 1,000 loci, between 600 and 1,200 loci, between 600 and 1,400 loci, between 600 and 1,600 loci, between 600 and 1,800 loci, between 600 and 2,000 loci, between 600 and 2,200 loci, between 600 and 2,400 loci, between 600 and 2,600 loci, between 600 and 2,800 loci, between 600, and 3,000 loci, between 800 and 1,000 loci, between 800 and 1,200 loci, between 800 and 1,400 loci, between 800 and 1,600 loci, between 800 and 1,800 loci, between 800 and 2,000 loci, between 800 and 2,200 loci, between 800 and 2,400 loci, between 800 and 2,600 loci, between 800 and 2,800 loci, between 800, and 3,000 loci, between 1,000 and 1,200 loci, between 1,000 and 1,400 loci, between 1,000 and 1,600 loci, between 1,000 and 1,800 loci, between 1,000 and 2,000 loci, between 1,000 and 2,200 loci, between 1,000 and 2,400 loci, between 1,000 and 2,600 loci, between 1,000 and 2,800 loci, between 1,000, and 3,000 loci, between 1,200 and 1,400 loci, between 1,200 and 1,600 loci, between 1,200 and 1,800 loci, between 1,200 and 2,000 loci, between 1,200 and 2,200 loci, between 1,200 and 2,400 loci, between 1,200 and 2,600 loci, between 1,200 and 2,800 loci, between 1,200, and 3,000 loci, between 1,400 and 1,600 loci, between 1,400 and 1,800 loci, between 1,400 and 2,000 loci, between 1,400 and 2,200 loci, between 1,400 and 2,400 loci, between 1,400 and 2,600 loci, between 1,400 and 2,800 loci, between 1,400, and 3,000 loci, between 1,600 and 1,800 loci, between 1,600 and 2,000 loci, between 1,600 and 2,200 loci, between 1,600 and 2,400 loci, between 1,600 and 2,600 loci, between 1,600 and 2,800 loci, between 1,600, and 3,000 loci, between 1,800 and 2,000 loci, between 1,800 and 2,200 loci, between 1,800 and 2,400 loci, between 1,800 and 2,600 loci, between 1,800 and 2,800 loci, between 1,800, and 3,000 loci, between 2,000 and 2,200 loci, between 2,000 and 2,400 loci, between 2,000 and 2,600 loci, between 2,000 and 2,800 loci, between 2,000 and 3,000 loci, between 2,200 and 2,400 loci, between 2,200 and 2,600 loci, between 2,200 and 2,800 loci, between 2,200, and 3,000 loci, between 2,400 and 2,600 loci, between 2,400 and 2,800 loci, between 2,400, and 3,000 loci, between 2,600 and 2,800 loci, between 2,600, and 3,000 loci, or between 2,800 and 3,000 loci.
185. The method of any of embodiments 1-22 or embodiment 184, wherein a minimum coverage requirement is at least 75×, 100×, 150×, 150×, 200×, or 250×.
186. The method of any one of embodiments 1-22 or embodiments 184-185, further comprising displaying a user interface comprising the report via an online portal.
187. The method of any one of embodiments 1-22 or embodiments 184-186, further comprising displaying a user interface comprising the report via a mobile device.
188. The method of embodiment 61, wherein the cancer is a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of the oral cavity, cancer of the pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.
189. The method of any one of embodiments 23-72 or embodiment 188, further comprising selecting a cancer therapy to administer to the subject based on the presence of the genetic variant in the sample.
190. The method of embodiment 189, further comprising determining an effective amount of a cancer therapy to administer to the subject based on the presence of the genetic variant in the sample.
191. The method of embodiment 189 or embodiment 190, further comprising administering the cancer therapy to the subject based on the presence of the genetic variant in the sample.
192. The method of any one of embodiments 189-190, wherein the cancer therapy comprises chemotherapy, radiation therapy, immunotherapy, surgery, or a therapy configured to target the presence of the genetic variant in the sample.
193. A method of selecting a cancer therapy, the method comprising:
   responsive to determining the presence of the genetic variant in a sample from a subject, selecting a cancer therapy for the subject, wherein the presence of the genetic variant in the sample is determined according to the method of any one of embodiments 23-72 or embodiments 188-192.
194. A method of treating a cancer in a subject, comprising:
   responsive to determining the presence of the genetic variant in a sample from the subject, administering an effective amount of a cancer therapy to the subject, wherein the presence of the genetic variant in the sample is determined according to the method of any one of embodiments 23-72 or embodiments 188-192.
195. A method for monitoring tumor progression or recurrence in a subject, the method comprising:
   determining a first presence of the genetic variant in a first sample obtained from the subject at a first time point according to the method of any one of embodiments 23-72 or embodiments 188-192;
   determining a second presence of the genetic variant in a second sample obtained from the subject at a second time point; and
   comparing the first presence of the genetic variant to the second presence of the genetic variant, thereby monitoring the tumor progression or recurrence.
196. The method of embodiment 195, wherein the second presence of the genetic variant for the second sample is determined according to the method of any one of embodiments 23-72 or embodiments 188-192.
197. The method of embodiment 195 or embodiment 196, further comprising adjusting a tumor therapy in response to the tumor progression.
198. The method of any one of embodiments 195-197, further comprising adjusting a dosage of the tumor therapy or selecting a different tumor therapy in response to the tumor progression.
199. The method of embodiment 198, further comprising administering the adjusted tumor therapy to the subject.
200. The method of any one of embodiments 195-199, wherein the first time point is before the subject has been administered a tumor therapy, and wherein the second time point is after the subject has been administered the tumor therapy.
201. The method of any one of embodiments 195-200, wherein the subject has a cancer, is at risk of having a cancer, is being routine tested for cancer, or is suspected of having a cancer.
202. The method of any one of embodiments 195-201, wherein the cancer is a solid tumor.
203. The method of any one of embodiments 195-202, wherein the cancer is a hematological cancer.
204. The method of embodiment 69, wherein the genomic profile for the subject further comprises results from a comprehensive genomic profiling (CGP) test, a gene expression profiling test, a cancer hotspot panel test, a DNA methylation test, a DNA fragmentation test, an RNA fragmentation test, or any combination thereof.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

1. A method of detecting a genetic variant or determining a variant allele frequency in a sample from a subject, comprising:
providing a plurality of nucleic acid molecules obtained from the sample;
ligating one or more adapters onto one or more nucleic acid molecules from the plurality of nucleic acid molecules;
amplifying the one or more ligated nucleic acid molecules from the plurality of nucleic acid molecules;
capturing the amplified nucleic acid molecules from the amplified nucleic acid molecules;
sequencing, by a sequencer, the captured nucleic acid molecules to obtain a plurality of sequencing reads that represent the nucleic acid molecules, wherein one or more of the plurality of sequencing reads overlap a variant locus of the genetic variant;
generating, using one or more processors, a reference match score for each of the one or more sequencing reads by aligning each of the one or more sequencing reads to a reference sequence that does not comprise the genetic variant;
generating, using the one or more processors, a variant match score for each of the one or more sequencing reads by aligning each sequencing read to a variant sequence that comprises the genetic variant;
based on the reference match score and the variant match score of a respective sequencing read, labeling, using the one or more processors, each of the one or more sequencing reads as at least one of having the genetic variant, not having the genetic variant, or being an inconclusive read;
determining, using the one or more processors, a number of sequencing reads labeled as having the genetic variant in the plurality of sequencing reads;
determining, using the one or more processors, a probability metric based on a variant specific model, the number of sequencing reads labeled as having the genetic variant, and a total number of labeled sequencing reads; and
identifying, using the one or more processors, the presence of the genetic variant in the sample when the determined probability metric is less than a first threshold.

2.-23. (canceled)

24. The method of claim 1, wherein the variant specific model is locus specific.

25. The method of claim 1, wherein the first threshold is locus specific and variant specific.

26. The method of claim 1, wherein the probability metric corresponds to a probability that the genetic variant is detected due to the presence of the genetic variant in the sample rather than noise.

27. The method of claim 1, further comprising comparing, using the one or more processors, the determined probability metric to a second threshold, and:
identifying, by the one or more processors, the absence of the genetic variant in the sample if the determined probability metric is greater than or equal to the second threshold; or
identifying, by the one or more processors, the presence or absence of the genetic variant in the sample as inconclusive if the determined probability metric is greater than or equal to the first threshold and less than the second threshold.

28. The method of claim 1, wherein the variant specific model is generated by:
fitting, using the one or more processors, a probability distribution based on a determined metric and a total number of labeled sequencing reads from a wild-type sample.

29. The method of claim 28, wherein the probability distribution is a binomial distribution.

30. The method of claim 1, wherein the probability metric is determined from the number of sequencing reads labeled as having the genetic variant and a second number, wherein the second number is the total number of labeled sequencing reads minus a number of sequencing reads labeled as being inconclusive reads.

31. The method of claim 1, wherein the variant specific model is associated with one or more functions related to one of more sources of noise in a plurality of sequencing reads that overlap the variant locus.

32. (canceled)

33. The method of claim 1, wherein the variant specific model is associated with one or more functions that have been fitted to data for a plurality of sequencing reads that overlap the variant locus, and wherein the one or more functions comprise one or more of uniform distribution functions, binomial distribution functions, Poisson distribution functions, negative binomial distribution functions, normal distribution functions, log-normal distribution functions, Cauchy-Lorentz distribution functions, log-logistic distribution functions, exponential distribution functions, gamma distribution functions, hypergeometric distribution functions, or any combination thereof.

34.-35. (canceled)

36. The method of claim 1, wherein a sequencing read is labeled as not having the genetic variant if the reference match score and the variant match score indicate that the sequencing read more closely matches the reference sequence than the variant sequence.

37. The method of claim 1, wherein a sequencing read is labeled as the inconclusive read if the reference match score and the variant match score are equal.

38. The method of claim 1, wherein the first threshold is determined empirically using the variant specific model.

39. The method of claim 1, wherein at least one of the first threshold or the second threshold is determined empirically using clinical trial outcomes.

40. The method of claim 1, wherein the first threshold is determined using a Kaplan-Meier estimator and data associated with samples from a plurality of subjects.

41. The method of claim 39, wherein the second threshold is determined empirically using the variant specific model, and is set to a value that corresponds to a specified confidence level that a sequencing read labeled as not containing the genetic variant is correct.

42. The method of claim 1, wherein the reference sequence and the variant sequence comprise the variant locus, a 5′ flanking region, and a 3′ flanking region.

43.-45. (canceled)

46. The method of claim 1, wherein the reference sequence and the variant sequence are substantially identical except for the genetic variant.

47. The method of claim 1, comprising determining a variant allele frequency for the genetic variant using the number of sequencing reads labeled as having the genetic variant and the number of sequencing reads labeled as not having the genetic variant.

48.-50. (canceled)

51. The method of claim 1, comprising determining a disease status for the subject.

52. The method of claim 51, wherein the disease status is a value proportional to a percentage of circulating-tumor DNA (ctDNA) compared to total cell-free DNA (cfDNA) in the sample.

53. The method of claim 52, wherein the disease status is a maximum somatic allele fraction of cfDNA.

54. The method of claim 52, wherein the disease status comprises a qualitative factor indicating recurrence of a cancer in the subject, the presence of a cancer resistant to a treatment modality in the subject, or the presence of a cancer that can be treated with a particular treatment modality.

55. The method of claim 1, wherein the sample comprises cfDNA.

56. The method of claim 1, wherein the reference match score and the variant match score are determined using a sequence alignment algorithm.

57. (canceled)

58. The method of claim 1, wherein the genetic variant comprises a single nucleotide variant (SNV), a multiple nucleotide variant (MNV), an indel, or a rearrangement junction.

59.-69. (canceled)

70. The method of claim 1, wherein the presence of the genetic variant of the sample is used in making suggested treatment decisions for the subject.

71. The method of claim 1, wherein the presence of the genetic variant of the sample is used in applying or administering a treatment to the subject.

72.-79. (canceled)

80. The method of claim 1, wherein the sample is derived from a liquid biopsy sample from the subject.

81.-191. (canceled)

192. The method of claim 70, wherein the treatment comprises chemotherapy, radiation therapy, immunotherapy, surgery, or a therapy configured to target the presence of the genetic variant in the sample.

193.-204. (canceled)


SEQUENCE LISTING (6 sequences)

SEQ ID NO: 1 - DNA, 21 bp, Artificial Sequence
  Synthetic Construct
aatgatgcacatcatggtggc

SEQ ID NO: 2 - DNA, 21 bp, Artificial Sequence
  Synthetic Construct
aatgatgcacgtcatggtggc

SEQ ID NO: 3 - DNA, 41 bp, Artificial Sequence
  Synthetic Construct
tggagtatttcatgaaacaaatgaatgatgcacatcatggt

SEQ ID NO: 4 - DNA, 59 bp, Artificial Sequence
  Synthetic Construct
aaacaaatgaatgatgcacatcatggtggctggacaacaaaaatggattggatcttcca

SEQ ID NO: 5 - DNA, 69 bp, Artificial Sequence
  Synthetic Construct
tcatgaaacaaatgaatgatgcacgtcatggtggctggacaacaaaaatggattggatcttccacacaa

SEQ ID NO: 6 - DNA, 68 bp, Artificial Sequence
  Synthetic Construct
  misc_feature 29, 39: n = A,T,C or G
tatttcatgaaacaaatgaatgatgcacntcatggtggntggacaacaaaaatggattggatcttcca